SEQUENCE LISTING (67 sequences)

SEQ ID NO: 1 - DNA, 19 bp, Artificial Sequence
  Synthetic primer
aacmggattagataccckg

SEQ ID NO: 2 - DNA, 19 bp, Artificial Sequence
  Synthetic primer
ggttaccttgttacgactt

SEQ ID NO: 3 - DNA, 20 bp, Artificial Sequence
  Synthetic primer
tcctccgcttattgatatgc

SEQ ID NO: 4 - DNA, 22 bp, Artificial Sequence
  Synthetic primer
cttggtcatttagaggaagtaa

SEQ ID NO: 5 - DNA, 659 bp, Paenibacillus sp.
  misc_feature: EKM101P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctttgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaggagcgatctggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtc

SEQ ID NO: 6 - DNA, 629 bp, Paenibacillus sp.
  misc_feature: EKM203B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaa

SEQ ID NO: 7 - DNA, 660 bp, Paenibacillus sp.
  misc_feature: EKM211P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtcg

SEQ ID NO: 8 - DNA, 648 bp, Paenibacillus sp.
  misc_feature: EKM213B
taaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtg

SEQ ID NO: 9 - DNA, 676 bp, Paenibacillus sp.
  misc_feature: EKM102P
taggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctttgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaggagcgatgtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtcgta

SEQ ID NO: 10 - DNA, 658 bp, Paenibacillus sp.
  misc_feature: EKM204P
cgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagataggcctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaggcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtcg

SEQ ID NO: 11 - DNA, 657 bp, Paenibacillus sp.
  misc_feature: EKM204B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcg

SEQ ID NO: 12 - DNA, 682 bp, Paenibacillus sp.
  misc_feature: EKM212P
gctaggtgttaggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcgtaac

SEQ ID NO: 13 - DNA, 658 bp, Paenibacillus sp.
  misc_feature: EKM214B
taaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagg

SEQ ID NO: 14 - DNA, 662 bp, Paenibacillus sp.
  misc_feature: EKM101P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctttgaccggtctagagataggcctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtcgta

SEQ ID NO: 15 - DNA, 621 bp, Paenibacillus sp.
  misc_feature: EKM205P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatatggctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaaggga

SEQ ID NO: 16 - DNA, 667 bp, Paenibacillus sp.
  misc_feature: EKM205B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaacaaggta

SEQ ID NO: 17 - DNA, 499 bp, Paenibacillus sp.
  misc_feature: EKM302P
tgaccggtctagagataggcctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatgattggggtgaagtcgta

SEQ ID NO: 18 - DNA, 632 bp, Paenibacillus sp.
  misc_feature: EKM206P
tttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtgtagagatatggctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgcc

SEQ ID NO: 19 - DNA, 586 bp, Paenibacillus sp.
  misc_feature: EKM206B
ccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaa

SEQ ID NO: 20 - DNA, 604 bp, Paenibacillus sp.
  misc_feature: EKM207P
tgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaagg

SEQ ID NO: 21 - DNA, 656 bp, Paenibacillus sp.
  misc_feature: EKM207B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtc

SEQ ID NO: 22 - DNA, 669 bp, Paenibacillus sp.
  misc_feature: EKM208P
taggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagataggcctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaaatcgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtggggtaacccgcaagggagccagccgccgaaggtggggtagatgattggggtgaagtcg

SEQ ID NO: 23 - DNA, 618 bp, Paenibacillus sp.
  misc_feature: EKM201P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaagg

SEQ ID NO: 24 - DNA, 611 bp, Paenibacillus sp.
  misc_feature: EKM208B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttt

SEQ ID NO: 25 - DNA, 625 bp, Paenibacillus sp.
  misc_feature: EKM202P
taggggtttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagacctttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaagg

SEQ ID NO: 26 - DNA, 646 bp, Paenibacillus sp.
  misc_feature: EKM101P
  misc_feature: EKM210P
ttcgatacccttggtgccgaagttaacacattaagcattccgcctggggagtacggtcgcaagactgaaactcaaaggaattgacggggacccgcacaagcagtggagtatgtggtttaattcgaagcaacgcgaagaaccttaccaggtcttgacatccctctgaccggtctagagatagrcytttccttcgggacagaggagacaggtggtgcatggttgtcgtcagctcgtgtcgtgagatgttgggttaagtcccgcaacgagcgcaacccttatgcttagttgccagcaggtcaagctgggcactctaagcagactgccggtgacaaaccggaggaaggtggggatgacgtcaaatcatcatgccccttatgacctgggctacacacgtactacaatggccggtacaacgggaagcgaagccgcgaggtggagccaatcctagaaaagccggtctcagttcggattgtaggctgcaactcgcctacatgaagtcggaattgctagtaatcgcggatcagcatgccgcggtgaatacgttcccgggtcttgtacacaccgcccgtcacaccacgagagtttacaacacccgaagtcggtgaggtaaccgcaaggagccagccgccgaaggtggggtagatga

SEQ ID NO: 27 - DNA, 671 bp, Paenibacillus sp.
  misc_feature: EKM210B
tagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcg

SEQ ID NO: 28 - DNA, 404 bp, Paenibacillus sp.
  misc_feature: EKM103A
cccgtaacgagcgcaacccttgtccttagttaccagcacgtaatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgt

SEQ ID NO: 29 - DNA, 618 bp, Paenibacillus sp.
  misc_feature: EKM403A
gttgggagtcttgactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaacc

SEQ ID NO: 30 - DNA, 661 bp, Paenibacillus sp.
  misc_feature: EKM410B
ttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaac

SEQ ID NO: 31 - DNA, 671 bp, Paenibacillus sp.
  misc_feature: EKM211B
tagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacaa

SEQ ID NO: 32 - DNA, 585 bp, Paenibacillus sp.
  misc_feature: EKM501U
cattccacggtttccgcgccgcagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatgttctcgatcgccgtagagatacggtttcccctttggggcgggttcacaggtggtgcatggttgtcgtcagctcgtgtcgtgagatgttgggttaagtcccgcaacgagcgcaaccctcgttccatgttgccagcacgtcatggtggggactcatgggagactgccggggtcaactcggaggaaggtgaggacgacgtcaaatcatcatgccccttatgtcttgggcttcacgcatgctacaatggccggtacaatgggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagt

SEQ ID NO: 33 - DNA, 665 bp, Paenibacillus sp.
  misc_feature: EKM102Z
tgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggta

SEQ ID NO: 34 - DNA, 669 bp, Paenibacillus sp.
  misc_feature: EKM505B
tagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttaggagccagccgccgaaggtgggacagatgattggggtgaagtcgtaac

SEQ ID NO: 35 - DNA, 654 bp, Paenibacillus sp.
  misc_feature: EKM101T
cggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtc

SEQ ID NO: 36 - DNA, 670 bp, Paenibacillus sp.
  misc_feature: EKM201S
tagggggtttccgccccttagtgctgcagctaacgcattaagcactccgcctggggagtacgaccgcaaggttgaaactcaaaggaattgacggggacccgcacaagcggtggagcatgtggtttaattcgaagcaacgcgaagaaccttaccaaatcttgacatcctctgacccctctagagatagagttttccccttcgggggacagagtgacaggtggtgcatggttgtcgtcagctcgtgtcgtgagatgttgggttaagtcccgcaacgagcgcaacccttaagcttagttgccatcattaagttgggcactctaagttgactgccggtgacaaaccggaggaaggtggggatgacgtcaaatcatcatgccccttatgatttgggctacacacgtgctacaatggacaatacaaagggtagcgaaaccgcgaggtcaagcaaatcccataaagttgttctcagttcggattgtagtctgcaactcgactacatgaagctggaatcgctagtaatcgtagatcagcatgctacggtgaatacgttcccgggtcttgtacacaccgcccgtcacaccacgagagtttgtaacacccgaagccggtggagtaaccttttggagctagccgtcgaaggtgggacaaatgattggggtgaagtcgtaac

SEQ ID NO: 37 - DNA, 665 bp, Paenibacillus sp.
  misc_feature: EKM306B
tagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtc

SEQ ID NO: 38 - DNA, 655 bp, Paenibacillus sp.
  misc_feature: EKM126B
ttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtc

SEQ ID NO: 39 - DNA, 525 bp, Paenibacillus sp.
  misc_feature: EKM102L
aaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaaca

SEQ ID NO: 40 - DNA, 655 bp, Paenibacillus sp.
  misc_feature: EKM102T
cggtttccgtgacgcagctaacgcattaagttccccgcctggggagtacggccgcaaggctaaaactcaaaggaattgacggggacccgcacaagcggcggagcatgcggattaattcgatgcaacgcgaagaaccttaccaaggcttgacatatacgagaacgggccagaaatggtcaactctttggacactcgtaaacaggtggtgcatggttgtcgtcagctcgtgtcgtgagatgttgggttaagtcccgcaacgagcgcaaccctcgttctatgttgccagcacgtaatggtgggaactcatgggatactgccggggtcaactcggaggaaggtggggatgacgtcaaatcatcatgccccttatgtcttgggcttcacgcatgctacaatggccggtacaaagggctgcaataccgtgaggtggagcgaatcccaaaaagccggtcccagttcggattgaggtctgcaactcgacctcatgaagtcggagtcgctagtaatcgcagatcagcaacgctgcggtgaatacgttcccgggtcttgtacacaccgcccgtcaagtcatgaaagtcggtaacacctgaagccggtggcccaacccttgtggagggagccgtcgaaggtgggatcggtaattaggactaagtcg

SEQ ID NO: 41 - DNA, 637 bp, Paenibacillus sp.
  misc_feature: EKM305P
aggggtttcgatacccttggtgccgaagttaacacattaagcactccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttatatttagttgccagcayttcggrtgggcactctagatagactgccggtgacaaaccggaggaaggtggggatgacgtcaaatcatcatgccccttatgacctgggctacacacgtactacaatggccggtacaacgggctgcgaaatcgcgagatggagccaatcccaacaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaa

SEQ ID NO: 42 - DNA, 656 bp, Paenibacillus sp.
  misc_feature: EKM301Z
ccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagt

SEQ ID NO: 43 - DNA, 666 bp, Paenibacillus sp.
  misc_feature: EKM101V
gttccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaaca

SEQ ID NO: 44 - DNA, 668 bp, Paenibacillus sp.
  misc_feature: EKM702B
gttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgt

SEQ ID NO: 45 - DNA, 668 bp, Paenibacillus sp.
  misc_feature: EKM503B
tagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaa

SEQ ID NO: 46 - DNA, 636 bp, Paenibacillus sp.
  misc_feature: EKM306P
ggggtttcgatacccttggtgccgaagttaacacattaagcactccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttatatttagttgccagcacttcggrtgggcactctagatagactgccggtgacaaaccggaggaaggtggggatgacgtcaaatcatcatgccccttatgacctgggctacacacgtactacaatggccggtacaacgggctgcgaaatcgcgagatggagccaatcccaacaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaa

SEQ ID NO: 47 - DNA, 660 bp, Paenibacillus sp.
  misc_feature: EKM301K
tgttccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagt

SEQ ID NO: 48 - DNA, 665 bp, Paenibacillus sp.
  misc_feature: EKM119B
tagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcg

SEQ ID NO: 49 - DNA, 428 bp, Paenibacillus sp.
  misc_feature: EKM303P
agacaggtggtgcatggttgtcgtcagctcgtgtcgtgagatgttgggttaagtcccgcaacgagcgcaacccttgatcttagttgccagcatttcggrtgggcactctaaggtgactgccggtgacaaaccggaggaaggtggggatgacgtcaaatcatcatgccccttatgacctgggctacacacgtactacaatggccggtacaacgggctgtgaagccgcgaggtggaacgaatcctaaaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaa

SEQ ID NO: 50 - DNA, 671 bp, Paenibacillus sp.
  misc_feature: EKM703B
tagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcgagaccgcaaggtttagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgcaacacccgaagtcggtgaggtaacctttatggagccagccgccgaaggtggggcagatgattggggtgaagtcg

SEQ ID NO: 51 - DNA, 659 bp, Paenibacillus sp.
  misc_feature: EKM501M
caaccctcggtgctgtcgctaacgcaataagcactccgcctggggagtacggccgcaaggctgaaactcaaaggaattgacggggacccgcacaagcggtggagcatgtggtttaattcgaagcaacgcgaagaaccttaccaaggcttgacatccccctgatcggtctagagatagatctttcccttcggggacaggggtgacaggtggtgcatggttgtcgtcagctcgtgtcgtgagatgttgggttaagtcccgcaacgagcgcaacccttatctttagttgccagcattcagttgggcactctagagagactgccggtgacaaaccggaggaaggtggggatgacgtcaaatcatcatgccccttatgtcttgggctacacacgtgctacaatggttggtacaaagggcagcaagccagcgatggtaagcaaatcccagaaagccaatctcagttcggattgtaggctgcaactcgcctacatgaagtcggaatcgctagtaatcgcagatcagcatgctgcggtgaatacgttcccgggtcttgtacacaccgcccgtcacaccacgagagtttgcaacacccgaagtcggtgaggtaaccgcaaggagccagccgccgaaggtggggcagatgattggggtgaagtcgtaac

SEQ ID NO: 52 - DNA, 657 bp, Paenibacillus sp.
  misc_feature: EKM602Z
cttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcg

SEQ ID NO: 53 - DNA, 658 bp, Paenibacillus sp.
  misc_feature: EKM302K
tccttgaggagtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccagtgataaactggaggaaggtggggatgacgtcaagtcatcatggcccttacgagtagggctacacacgtgctacaatggcatatacaaagagaagcgacctcgcgagagcaagcggacctcataaagtatgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtc

SEQ ID NO: 54 - DNA, 578 bp, Paenibacillus sp.
  misc_feature: EKM103V
gggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgcatacaaagagaagcgacctcgcgagagcaagcggacctcacaaagtgcgtcgtagtccggatcggagtctgcaactcgactccgtgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtacaa

SEQ ID NO: 55 - DNA, 664 bp, Paenibacillus sp.
  misc_feature: EKM203L
gtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtc

SEQ ID NO: 56 - DNA, 623 bp, Paenibacillus sp.
  misc_feature: EKM102E
gaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcggttaggccgggaactcaaaggagactgccagtgataaactggaggaaggtggggatgacgtcaagtcatcatggcccttacgagtagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgta

SEQ ID NO: 57 - DNA, 666 bp, Paenibacillus sp.
  misc_feature: EKM402A
  misc_feature (620)..(620): n is a, c, g, or t
cgttgggagtcttgactcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccntcgggaggacggttaccacggtgtgattcatgactggggtgaagtc

SEQ ID NO: 58 - DNA, 672 bp, Paenibacillus sp.
  misc_feature: EKM308P
taggtgttaggggtttcgatacccttggtgccgaagttaacacattaagcactccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttatatttagttgccagcacttcggrtgggcactctagatagactgccggtgacaaaccggaggaaggtggggatgacgtcaaatcatcatgccccttatgacctgggctacacacgtactacaatggccggtacaacgggctgcgaaatcgcgagatggagccaatcccaacaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaaggtgggatagatgattggggtgaagtc

SEQ ID NO: 59 - DNA, 664 bp, Paenibacillus sp.
  misc_feature: EKM312P
taggggtttcgatacccttggtgccgaagttaacacattaagcactccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttatatttagttgccagcacttcggrtgggcactctagatagactgccggtgacaaaccggaggaaggtggggatgacgtcaaatcatcatgccccttatgacctgggctacacacgtactacaatggccggtacaacgggctgcgaaatcgcgagatggagccaatcccaacaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaaggtgggatagatgattggggtgaagt

SEQ ID NO: 60 - DNA, 641 bp, Paenibacillus sp.
  misc_feature: EKM201D
gtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaa

SEQ ID NO: 61 - DNA, 668 bp, Paenibacillus sp.
  misc_feature: EKM502B
gatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcg

SEQ ID NO: 62 - DNA, 657 bp, Paenibacillus sp.
  misc_feature: EKM101P
ttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacactcctagagataggaatttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggcagcaaagccgcgaggcctagccaatcccataaaaccattctcagttcggattgtaggctgcaactcgcctacatgaagccggaatcgctagtaatcgcggatcagcatgccgcggtgaatacgttcccgggccttgtacacaccgcccgtcacaccacgagagtttgtaacacccgaagtcggtggggtaaccgtaaggagccagccgcctaaggtgggacagatgattggggtgaagtc

SEQ ID NO: 63 - DNA, 661 bp, Paenibacillus sp.
  misc_feature: EKM417B
tagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttatggagccagccgccgaaggtgggacagatgattggggtgaa

SEQ ID NO: 64 - DNA, 643 bp, Paenibacillus sp.
  misc_feature: EKM202D
gtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgacagtctaagagattagaggttcccttcggggacagaatgacaggtggtgcatggttgtcgtcagctcgtgtcgtgagatgttgggttaagtcccgcaacgagcgcaacccttattactagttgccagcattaagttgggcactctagtgagactgccggtgacaaaccggaggaaggtggggacgacgtcaaatcatcatgccccttatgacctgggctacacacgtgctacaatggatggtacaacgagtcgcgagaccgcgaggttaagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaaccttttaggagctagccgtctaaggtgggacagatgattagggtgaagt

SEQ ID NO: 65 - DNA, 669 bp, Paenibacillus sp.
  misc_feature: EKM502L
gtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgaagaaccttaccaggtcttgacatactcgtgctattcctagagataggaagttccttcgggacacgggatacaggtggtgcatggttgtcgtcagctcgtgtcgtgagatgttgggttaagtcccgcaacgagcgcaacccctattgttagttgccatcattaagttgggcactctaacgagactgccggtgataaaccggaggaaggtggggatgacgtcaaatcatcatgccccttatgacctgggctacacacgtgctacaatggatggtacaacgagtcgcgagacagtgatgtttagctaatctcttaaaaccattctcagttcggattgtaggctgcaactcgcctacatgaagtcggaatcgctagtaatcgcggatcagcacgccgcggtgaatacgttcccgggccttgtacacaccgcccgtcacaccacgggagttgggagtacccgaagtaggttgcctaaccgcaaggagggcgcttcctaaggtaagaccgatgactggggtgaagtcgtaac

SEQ ID NO: 66 - DNA, 670 bp, Paenibacillus sp.
  misc_feature: EKM419B
  misc_feature (25)..(25): n is a, c, g, or t
tagtgttagagggtttccgcccttnagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagt

SEQ ID NO: 67 - DNA, 667 bp, Paenibacillus sp.
  misc_feature: EKM310P
gttaggggtttcgatacccttggtgccgaagttaacacattaagcactccgcctggggagtacggtcgcaagactgaaactcaaaggaattgacggggacccgcacaagcagtggagtatgtggtttaattcgaagcaacgcgaagaaccttaccaggtcttgacatccctctgaccggtacagagatgtacctttccttcgggacagaggagacaggtggtgcatggttgtcgtcagctcgtgtcgtgagatgttgggttaagtcccgcaacgagcgcaacccttatatttagttgccagcacttcgggtgggcactctagatagactgccggtgacaaaccggaggaaggtggggatgacgtcaaatcatcatgccccttatgacctgggctacacacgtactacaatggccggtacaacgggctgcgaaatcgcgagatggagccaatcccaacaaagccggtctcagttcggattgcaggctgcaactcgcctgcatgaagtcggaattgctagtaatcgcggatcagcatgccgcggtgaatacgttcccgggtcttgtacacaccgcccgtcacaccacgagagtttataacacccgaagtcggtggggtaaccgcaaggagccagccgccgaaggtgggatagatgattgggggtgaagt

(19) United States
(12) Patent Application Publication
(10) Pub. No.: US 2017/0238552 A1
(43) Pub. Date: Aug. 24, 2017

(54) BACTERIAL ENDOPHYTES FROM CUCURBIT SPECIES

(71) Applicant: University of Guelph, Guelph (CA)

(72) Inventors: Manish N. Raizada, Milton (CA); Eman M. Khalaf, Guelph (CA)

(21) Appl. No.: 15/422,005

(22) Filed: Feb. 1, 2017

Related U.S. Application Data
(60) Provisional application No. 62/289,737, filed on Feb. 1, 2016.

(51) Int. Cl. A01N 63/00; C12N 1/20

(52) U.S. Cl. CPC A01N 63/00; C12N 1/20

(57) ABSTRACT

Bacterial endophytes isolated from cucurbit species are described. The endophytes are useful in preventing or inhibiting the growth of pathogens on a plant, in particular preventing or inhibiting the growth of fungus and/or powdery mildew. The endophytes are also useful for improving the growth characteristics of a plant.

### RELATED APPLICATIONS

This application claims the benefit of priority of U.S. Provisional Application Ser. No. 62/289,737 filed Feb. 1, 2016, the entire contents of which are hereby incorporated by reference.

### INCORPORATION OF SEQUENCE LISTING

A computer readable form of the Sequence Listing “6580-P50241 US01_SequenceListing.txt” (65,536 bytes), submitted via EFS-WEB and created on Feb. 1, 2017, is herein incorporated by reference.

### FIELD

The present disclosure relates to the bacterial endophytes and more specifically to bacterial endophytes from cucurbit species.

### BACKGROUND

Plants as metaorganisms are fundamentally associated with diverse microbes spanning different parts (such as rhizosphere, phylosphere and endosphere) and organs comprising vegetative organs (such as roots, stems and leaves) and reproductive and disseminating organs (such as flowers, fruits and seeds) of the host plant [1], [2], [3], [4], [5]. Amongst beneficial microbes, the term “endophyte” refers to microbes that reside in internal tissues of plants without showing any visible adverse effects on their host [6], [7], [8]. Either culture-dependent and/or—independent techniques emphasize the diversity of bacterial endophytes to encompass various bacterial taxa that are intimately associated with wide-range of different plant species [5]. Evidence suggests that endophytes originate from the rhizosphere (soil) and/or through maternal inheritance to future generations (e.g. vertical transmission through seeds) [9], [10]. The hologenome theory of evolution postulates that the host and its associated beneficial microbiota co-evolve as one unit to provide benefits to one another including nutrient acquisition, growth promotion and defense [11]. In particular, bacteria, including endophytes, can assist with plant nutrient acquisition through biological nitrogen fixation, mineral phosphate solubilisation and production of siderophores [8], [12], [13]. Several endophytes have the capacity to stimulate plant growth either directly or indirectly. The direct route involves microbial production of phytohormones such as auxin (indole-3-acetic acid, IAA) which is a major plant growth stimulator, including of nutrient-acquiring roots [13]. The indirect route includes the reduction of levels of the plant stress hormone, ethylene, which signals suppression of plant growth to conserve nutrients. Microbes can degrade 1-aminocyclopropane-1-carboxylic acid (ACC), the precursor of plant hormone ethylene, via the enzyme ACC deaminase, resulting in stimulation of plant growth, especially roots [14].

The above nutrient and plant growth activities require colonization and community formation on plant surfaces [15]. For bacterial penetration into plant tissues, microbes secrete plant cell wall hydrolyzing enzymes such as pectinases and cellulases [8]. Microbial community formation can be facilitated by biofilms [15]; the biofilm matrix is composed of extracellular polymeric substances (EPS) comprising polysaccharides, proteins, nucleic acids and lipids. Biofilms confine microbially-secreted extracellular enzymes such as proteases (protein degrading enzymes), endocellulases and chitinases (polysaccharide degrading enzymes). These enzymes are responsible for breaking down their biopolymer substrates as energy sources [16]. Aside from their potential impacts on their host plant, seed associated microbes play a substantial role in the plant life cycle. First, they can assist with seed health and make them ready for germination [17]. Second, these microbes act as founders for the bacterial communities of the newly developed plant where they can provide benefits to their hosts. Most important, for multi-generational maintenance of these beneficial associations, plants can also use seeds as microbial vectors [4].

The gourd family Cucurbitaceae is classified into 15 tribes encompassing 97 genera and 940-980 species. They are mainly tropical and subtropical crops showing the highest diversity in these climates with hotspots in Southeast Asia, West Africa, Madagascar, and Mexico. However, few species can grow in temperate regions [18], [19], [20]. The family became nutritionally and culturally important to humans more than 12,000 years ago [21]. Today, based on their economic importance, three genera are of significant value within this family: Cucumis, Citrullus, and Cucurbita representing commonly consumed species, such as Cucumis sativus. L (cucmber), Cucumis melo. L (melon), Citrullus lanatus (Thunb.) Matsumura & Nakai (watermelon), and Cucurbita L. (squash and pumpkin), in addition to regionally important genera such as Luffa and Lagenaria [19], [22], [23]. According to Shaefer and Renner recent taxonomy of the gourd family, these five genera belong to three tribes (Table 1); Benincaseae (includes three genera; Citrullus, Lagenaria (n=11) and Cucumis (n=7 or 12)), Cucurbiteae (includes the genus Cucurbita (n=20)) and the genus Luffa (n=13) is classified under the tribe Sicyoeae [19]. Molecular studies based on chloroplast gene, spacer and intron sequences, suggest an Asian origin of the Cucurbitaceae family, followed by 43 long distance dispersal events over a 60 million year period to different continents [24].

Despite the enormous diversity and economic importance of the Cucurbitaceae family to humans and the importance of seeds as vectors for endophytes, there are few reports focused on the discovery of seed bacterial endophytes across this nutritionally important plant family. Recent research in Austria reported the diversity of bacterial endophytes colonizing seeds (spermosphere), roots (endorhiza), flowers (anthosphere), and fruits (carposphere) of three different pumpkin varieties and tested their potential application in combating pumpkin diseases [25], [26]. A publication by Glassner et al, 2015 was concerned with in situ visualization and cultivation of microbial communities associated with fruit flesh tissues and placenta (seed cavity without seeds) of selected species and/or varieties of melon, wild melon, watermelon, colocynth and bryony [5].

In contrast, various studies focused on studying the seed associated microbiome of other crop seeds have been reported. For example, in two consecutive rice seed generations, the diversity of rice seed associated bacterial endophytes and how the plant physiology dramatically influences the structure and diversity of the seed endophytic bacterial communities has been reported [27]. Furthermore, on the seed surface of various wheat (Triticum spp.) and canola (Brassica spp.) samples of diverse geographical origins, a conserved epiphytic microbiome was identified [28]. In the genus Zea, which includes corn (maize), seed endophytic communities were demonstrated to have co-evolved with their hosts, with a core microbiome that was conserved across domestication, migration and breeding events [29].

There remains a need for the identification of novel endophytes from Cucurbitaceae seeds and characterization of their activities related to plant growth promotion and nutrient acquisition.

### SUMMARY

Endophytes are microbes that colonize plant internal tissues without causing disease. In particular, seed-associated endophytes may be vectors for founder microbes that establish the plant microbiome, which may subsequently contribute beneficial functions to their host plants including nutrient acquisition and promotion of plant growth. The Cucurbitaceae family of gourds (e.g. cucumbers, melons, pumpkin, squash), including its fruits and seeds, is widely consumed by humans.

The present inventors isolated bacteria from surface-sterilized seeds of seven economically important cucurbit species. The isolated bacteria were cultured, classified using 16S rRNA (rDNA) sequencing, and subjected to in vitro functional tests including the production of the plant hormone, indole-3-acetic acid (auxin; known to stimulate the growth of fruits/gourds and nutrient-acquiring roots) as well as nitrogen fixation/N-scavenging, phosphate solubilisation, siderophore secretion, and production of ACC deaminase. Secretion of extracellular enzymes required for nutrient acquisition, endophyte colonization and/or community establishment were also observed. 16S rDNA sequences for the isolated bacterial endophytes identified in Table 2 are found SEQ ID NOs: 5-67

Remarkably, additional testing also demonstrated that a number of the isolated bacterial strains exhibited activity against plant pathogens. Bacterial strains having activity against plant pathogens are identified in Table 2. In particular, certain bacterial strains were shown to have activity against Fusarium graminearum, Rhizoctonia solani, Phytophthora capsici and/or Pythium aphanideratum as shown in Table 4. Testing also identified bacterial strains from cucurbit species with activity to biologically control powdery mildew (Podosphaera fuliginea) as shown in Tables 7-9, FIG. 7 and FIG. 8. Some of these bacterial strains with activity against powdery mildew also showed activity against Fusarium graminearum, Rhizoctonia solani, Phytophthora capsici and/or Pythium aphanideratum as shown in Table 6.

Accordingly, in one aspect, there is provided an isolated bacterial endophyte comprising a 16S rDNA gene comprising a nucleotide sequence with at least 90, 95%, 96%, 97%, 98%, 99% or 100% sequence identity to a sequence selected from SEQ ID NOs: 5-67, or its progeny, or mutants thereof. In one embodiment, the bacterial endophyte comprises a 16S rDNA gene comprising a nucleotide sequence selected from SEQ ID NOs: 5-67.

In one embodiment, the endophyte is Bacillus, Paenibacillus, Pediococcus, Lactococcus, Pseudomonas, Pantoea, Staphylococcus or Micrococcus. In one embodiment, the endophyte is Bacillus. In one embodiment, bacterial endophyte is of the genus Pediococcus and comprises a 16S rDNA gene comprising a nucleotide sequence with at least 95%, 98%, 99% or 100% sequence identity to SEQ ID NO: 60.

The bacterial endophytes described herein exhibit a number of characteristics that distinguish them from other bacteria and/or confer beneficial traits to plants colonized by the bacterial endophytes. In some embodiments, the endophyte grows on nitrogen-free media. In some embodiments, the endophyte has ACC deaminase activity, phosphate solubilization activity, cellulase activity, pectinase activity and/or protease activity. In some embodiments, the endophyte produces auxin and/or siderophores. In one embodiment, the endophytes promote activities related to plant growth and/or nutrient acquisition.

In one embodiment, the bacterial endophytes described herein may be isolated from a gourd family Cucurbitaceae species, such as those set out in Table 1. In one embodiment, the endophyte is isolated from a plant, or seed of a plant, listed in Table 1.

In one embodiment, the bacterial endophytes described herein inhibit the growth of a plant pathogen. In one embodiment, the plant pathogen is a fungus. In one embodiment, the plant pathogen Fusarium graminearum, Rhizoctonia solani, Phytophthora capsici or Pythium aphanideratum. In one embodiment, the plant pathogen is Podosphaera fulginea (powdery mildew). In some embodiments, the endophyte may have activity against two or more different pathogens.

As shown in Table 4, certain endophytes isolated from cucurbit species have activity against Fusarium graminearum. Accordingly, in one embodiment the endophyte comprises a 16S rDNA gene comprising a nucleotide sequence with at least 90%, 95%, 96%, 97%, 98%, 99% or 100% sequence identity to a sequence selected from SEQ ID NOs: 5-26 and the bacterial endophyte inhibits the growth of Fusarium graminearum.

As shown in Tables 7-9, certain endophytes isolated from cucurbit species have activity against the growth of powdery mildew. Accordingly, in one embodiment the endophyte comprises a 16S rDNA gene comprising a nucleotide sequence with at least 90%, 95%, 96%, 97%, 98%, 99% or 100% sequence identity to a sequence selected from SEQ ID NOs: 27-67 and the bacterial endophyte inhibits the growth of powdery mildew. In a preferred embodiment, the bacterial endophyte has activity against the growth of powdery mildew and is selected from strains EKM101V, EKM102Z, EKM103V, EKM201S, EKM301K, EKM201D, EKM126B, EKM417B, EKM210B, EKM501U, EKM502L, EKM505B, EKM419B, EKM305P and EKM306B identified in Table 2. In another preferred embodiment, the bacterial endophyte has activity against the growth of powdery mildew and is selected from EKM101V, EKM103V, EKM201S, EKM102Z, EKM301K, EKM419B, EKM505B and EKM201D identified in Table 2. In one embodiment, the bacterial endophyte is EKM201D and the endophyte inhibits the growth of powdery mildew, inhibits the growth of Rhizoctonia solani and/or Phytophthora capsici, and/or has phosphate solubilization activity.

Also provided are compositions comprising one or more bacterial endophytes as described herein. In one embodiment, the compositions comprise at least one of a carrier, tackifier, microbial stabilizer, fungicide, antibacterial agent, herbicide, nematicide, insecticide, plant growth regulator, rodenticide, dessicant, nutrient and culture media. In one embodiment, the carrier comprises an agricultural carrier, a surfactant or a seed coating agent. Optoinally, the composition is a formulation suitable for inoculating a plant such as a fluid or powder. In one embodiment, the composition comprises at least 10³, 10⁴, 10⁵, 10⁶, 10⁷, 10⁸ or 10⁹ colony forming units (CFU) per ml or gram of the composition. In one embodiment, the composition is a bacterial cell culture comprising between 10⁵ and 10⁹ colony forming units (CFU) per ml or per gram. In one embodiment, the composition comprises a concentration of CFU suitable for inoculating a plant using a foliar spray.

In one aspect, there is provided a method of improving nutrient acquisition and/or growth of a plant comprising inoculating the plant with a bacterial endophyte as described herein or a composition comprising a bacterial endophyte as described herein. In one embodiment, the composition comprises a carrier.

In another aspect, there is provided a method of preventing or inhibiting growth of a pathogen on a plant, comprising inoculating the plant with a bacterial endophyte as described herein or a composition comprising a bacterial endophyte as described herein. In one embodiment, the composition comprises a carrier. In one embodiment, the pathogen is powdery mildew and the bacterial endophyte is identified in one or more of Tables 7, 8 and 9. In one embodiment the bacterial endophyte is heterologous to the plant. IN one embodiment, the method comprises inoculating the plant with a bacterial endophyte selected from EKM101V, EKM102Z, EKM103V, EKM201S, EKM301K, EKM201D, EKM505B and EKM419B.

In one embodiment, the plant is an agricultural plant, optionally a fruit or vegetable. In one embodiment, the plant is a plant that can be colonized by one or more of the bacterial endophytes described herein. In one embodiment, the plant is a gourd family Cucurbitaceae species. In one embodiment, the plant is a cucumber and the bacterial endophyte prevents the growth of powdery mildew. In one embodiment, the plant is a grape, grain, tree fruit, leafy vegetable or ornamental and the bacterial endophyte prevents the growth of powdery mildew on the plant.

Different methods known in the art may be used to inoculate the plant with the bacterial endophytes described herein. In one embodiment, inoculating a plant comprises coating the seeds of the plant and/or exposing the plant to a spray. In one embodiment, inoculating a plant comprises foliar introduction or in-furrow introduction.

In one embodiment, the methods described herein are useful for preventing or inhibiting the growth of one or more fungal pathogens. In one embodiment, the pathogen is Fusarium graminearum, Rhizoctonia solani, Phytophthora capsici or Pythium aphanideratum. In one embodiment, the pathogen is Fusarium graminearum. In another embodiment, the pathogen is Podosphaera fulginea (powdery mildew) or another microorganism known to cause powdery mildew in a plant host.

In another aspect, there is provided a synthetic combination comprising a bacterial endophyte as described herein in association with a plurality of seeds or seedlings of a plant. In one embodiment, the endophyte is heterologous to the seeds or seedlings. In one embodiment, the endophyte is present in the synthetic combination in an amount effective to provide a benefit to the seeds or seedlings or plants derived from the seeds or seedlings. In one embodiment, the bacterial endophyte is present in the synthetic combination at a concentration of at least 10³, 10⁴, 10⁵, 10⁶, 10⁷, 10⁸ or 10⁹ CFU per seed, optionally between about 10⁴ and 10⁹ CFU per seed or between about 10⁴ and 10⁷ CFU per seed.

In one embodiment, the benefit is selected from the group consisting of improved plant growth, increased plant mass, and increased plant yield. In one embodiment, the endophyte has ACC deaminase activity, phosphate solubilization activity, cellulase activity, pectinase activity and/or protease activity. In one embodiment, the endophyte produces auxin and/or siderophores.

Optionally, the synthetic combination may also comprise a carrier, tackifier, microbial stabilizer, fungicide, antibacterial agent, herbicide, nematicide, insecticide, plant growth regulator, rodenticide, dessicant, nutrient and/or culture media.

In another aspect, there is provided a method for preparing an agricultural seed composition comprising contacting the surface of a plurality of seeds with a formulation comprising a bacterial endophyte as described herein. In one embodiment, the endophyte is present in the synthetic combination in an amount effective to provide a benefit to the seeds or plants derived from the seeds. In one embodiment, the endophyte is heterologous to the seeds. In one embodiment, the bacterial endophyte is present in the synthetic combination at a concentration sufficient to inhibit the growth of powdery mildew in a plant produced from the seed relative to a plant produced from a seed that is not in association with the bacterial endophyte grown under the same conditions.

Also provided is the use of the bacterial endophyte as described herein on a plant as a biological control agent. In one embodiment, the biological control agent is for inhibiting or preventing the growth of plant pathogens or for improving the growth, mass and/or yield of a plant.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF DRAWINGS

FIG. 1 shows the geographical origins of the economically important cucurbits used in this study as sources of endophytes. Triangles indicate the immediate seed sources. Dark circles indicate the hotspots of diversity of the Cucurbitaceae. The phylogenetic tree was constructed using PhyloT phylogenetic tree generator, based on NCBI taxonomy [38], [39].

FIG. 2 shows the collection of putative seed-associated endophytes cultured from cucurbits in this study. (A) Selected photos of cucurbit seed associated bacterial endophytes cultured on different agar media (R2A, LGI, PDA). (B) Distribution of isolated bacterial genera across tested cucurbit varieties. The yellow highlight refers to the number of isolates in each bacterial genus from a corresponding cucurbit variety. The asterisk denotes that the seed source is from fresh fruit.

FIG. 3 shows a diagrammatic sketch of the taxonomy of the endophytes based on phylum and class/order. Maximum Likelihood (ML) trees of putative seed-associated endophytes cultured from cucurbits based on bacterial 16S rRNA genes sequences were generated for endophytes from commercial curcubit seeds (dry) as well as for endophytes from fresh fruit seeds (data not shown).

FIGS. 4A and 4B shows Maximum Likelihood (ML) trees of the cultured Bacillus and Paenibacillus putative endophytes from cucurbit seeds based on 16S rRNA genes sequences. Bootstrap values are indicated above the branches. FIG. 4A ML tree of the Paenibacillus strains from commercial cucurbit seeds (dry). FIG. 4B ML tree of the Bacillus strains isolated from fresh fruit seeds. ML trees of Bacillus strains from commercial cucurbit seeds (dry) and Paenibacillus strains from fresh fruit seeds were also generated (data not shown).

FIG. 5 shows a summary of in vitro functional traits of putative seed-associated endophytes cultured from cucurbits, grouped by bacterial genus. (A) Selected photos for the in vitro phenotype screens used. (B) Distribution of in vitro functional traits of the endophytes grouped by bacterial genus. The yellow highlight refers to the most prevalent functional trait exhibited by the endophytes within a bacterial genus (row). The dark orange highlight refers to the most prevalent bacterial genus that displays a particular functional trait (column). The green highlight refers to the intersection of the two categories. LAB: lactic acid bacteria.

FIG. 6 shows a summary of in vitro functional traits of putative seed-associated endophytes cultured from cucurbits, grouped by their cucurbit host plant. The yellow highlight refers to the most prevalent functional trait exhibited by the endophytes within a cucurbit variety (row). The dark orange highlight refers to the most prevalent cucurbit variety that displays a particular functional trait by their seed-associated endophytes (column). The green highlight refers to the intersection of the two categories. The asterisk denotes that the seed source is from fresh fruit.

FIG. 7 shows in planta screening of cucurbit seed associated endophytic bacteria for antagonism towards cucumber powdery mildew disease using a leaf disc bioassay. (A) Positive control Prothioconazole (commercial chemical fungicide). (B) Positive control Bacillus subtilis strain QST 713 (commercial biocontrol agent). (C) Promising endophytic bacteria. (D) Negative control (LB broth amended with filter sterilized 0.01% Tween 20). (E) Graph chart collecting endophytic bacteria that displayed positive values of preventive effect (PE%) and their distribution across the host plants. Numbers between brackets displayed on the horizontal axis (Genera of endophytic bacteria) denote the total number of strains in each corresponding bacterial genus that showed positive results. Numbers between brackets displayed on the depth axis (Tested cucurbits) denote the total number of strains identified from each corresponding cucurbit crop and showed positive results.

FIG. 8 shows in planta screening of cultivated cucurbit seed endophytic bacteria against cucumber powdery mildew (Podosphaera fuliginea) using a detached whole leaf bioassay. (A) Positive control Prothioconazole (commercial chemical fungicide). (B) Positive control Bacillus subtilis strain QST 713 (commercial biocontrol agent). (C) Promising endophytic bacteria. (D) Negative control (LB broth amended with filter sterilized 0.01% Tween 20).

### DETAILED DESCRIPTION

The present disclosure relates to bacteria isolated from gourd family Cucurbitaceae species. In one embodiment, the bacteria are endophytes capable of promoting activities related to plant growth and/or nutrient acquisition. In one embodiment, the bacteria are endophytes capable of inhibiting the growth of one or more plant pathogens. In one embodiment, the plant pathogen is selected from fusarium or powdery mildew.

Each of the bacterial strains identified in Table 2 were isolated, characterized and subjected to 16S rRNA sequencing. As indicated in Tables 3-9, the isolated strains of bacteria exhibit a number of beneficial and/or distinguishing characteristics. In particular, the bacterial endophytes listed in Table 4 inhibit the growth of the plant pathogenic fungus Fusarium graminearum. The bacterial endophytes listed in Tables 7-9 inhibit the growth of the powdery mildew. In some embodiments, the bacterial endophytes inhibit the growth of two or more plant pathogens. In some embodiments, the bacterial endophytes exhibit one or more characteristics such as RNase activity, acetoin and/or diacetyl production, growth on Nitrogen-Free media, ACC deaminase activity, phosphate solubilization, auxin production, siderophore production, cellulase activity, Pectinase activity and/or protease activity.

### I. Definitions

The term “endophyte” as used herein refers to a class of microbial symbionts that reside within host plant roots, stems and/or leaves.

The term “inoculating a plant” with an endophyte, for example, as used herein refers to applying, contacting or infecting a plant (including its roots, stem, leaves or seeds) with an endophyte or a composition comprising an endophyte. The term “inoculated plant” refers to a plant to which an endophyte or a composition comprising an endophyte has been applied or contacted.

The term “plant” as used herein includes any member of the plant kingdom that can be colonized by a bacterial endophyte. In one embodiment, the plant is a vegetable. In one embodiment, the plant is a gourd cucurbit species, including but not limited to the species listed in Table 1. As used herein, the term “plant” includes parts of a plant such as roots, stems, leaves and/or seeds that can be colonized by a bacterial endophyte.

The present invention contemplates the use of “isolated” endophyte. As used herein, an isolated endophyte is an endophyte that is isolated from its native environment, and carries with it an inference that the isolation was carried out by the hand of man. An isolated endophyte is one that has been separated from at least some of the components with which it was previously associated (whether in nature or in an experimental setting).

In some embodiments, the invention uses endophytes that are heterologous to a seed or plant in making synthetic combinations or agricultural formulations. An endophyte is considered heterologous to the seed or plant if the seed or seedling that is unmodified (e.g., a seed or seedling that is not treated with a bacterial endophyte population described herein) does not contain detectable levels of the endophyte. For example, the invention contemplates the synthetic combinations of plants, seeds or seedlings of agricultural plants (e.g., agricultural grass plants) and an endophyte population, in which the endophyte population is heterologously disposed on the exterior surface of or within a tissue of the agricultural plant, seed or seedling in an amount effective to colonize the plant. An endophyte is considered heterologously disposed on the surface or within a plant (or tissue) when the endophyte is applied or disposed on the plant in a number that is not found on that plant before application of the endophyte. For example, a bacterial endophytic population that is disposed on an exterior surface or within the seed can be an endophytic bacterium that may be associated with the mature plant, but is not found on the surface of or within the seed. As such, an endophyte is deemed heterologous or heterologously disposed when applied on the plant that either does not naturally have the endophyte on its surface or within the particular tissue to which the endophyte is disposed, or does not naturally have the endophyte on its surface or within the particular tissue in the number that is being applied.

The term “inhibiting fungal growth in a plant” as used herein means decreasing amount of fungal growth on a plant, decreasing the speed of fungal growth on a plant, decreasing the severity of a fungal infection in a plant, decreasing the amount of diseased area of a plant, decreasing the percentage or number of infected seeds, decreasing the percentage or number of disease lesions on a plant decreasing the percentage of apparent fungal infection of a plant and/or treating or preventing fungal growth in a plant.

The term “yield” refers to biomass or seed or fruit weight, seed size, seed number per plant, seed number per unit area, bushels per acre, tons per acre, kilo per hectare, and/or carbohydrate yield.

The term “promoting plant growth” as used herein means that the plant or parts thereof (such as seeds and roots) have increased in size or mass compared to a control plant, or parts thereof, that has not been inoculated with the endophyte or as compared to a predetermined standard.

The term “symbiosis” and/or “symbiotic relationship” as used herein refer to a mutually beneficial interaction between two organisms including the interaction plants can have with bacteria. Similarly, the term “symbiont” as used herein refers to an organism in a symbiotic interaction.

The term “sequence identity” as used herein refers to the percentage of sequence identity between two nucleic acid and/or polypeptide sequences. To determine the sequence identity of two nucleic acid sequences, the sequences are aligned for optimal comparison purposes (e.g., gaps can be introduced in the sequence of a first nucleic acid sequence for optimal alignment with a second nucleic acid sequence). The nucleic acid residues at corresponding nucleic acid positions are then compared. When a position in the first sequence is occupied by the same nucleotide as the corresponding position in the second sequence, then the molecules are identical at that position. The sequence identity between the two sequences is a function of the number of identical positions shared by the sequences (i.e., % identity=number of identical overlapping positions/total number of positions×100%). In one embodiment, the two sequences are the same length. The determination of sequence identity between two sequences can also be accomplished using a mathematical algorithm.

A “synthetic combination” includes a combination of a plant, such as an agricultural plant, and an endophyte. The combination may be achieved, for example, by coating the surface of the seed of a plant, such as an agricultural plant, or plant tissues with an endophyte.

As used herein, the terms “a” or “an” in relation to an object mean a representative example from a collection of that object.

### II. Bacterial Endophytes and Compositions Thereof

Endophytes, microbes that live inside a plant without causing disease, can confer beneficial traits to their host such as promoting health or protecting against specific host pathogens.

As set out in Example 1, the bacterial endophytes listed in Table 2 have been isolated from cucurbit species and characterized. The 16S rRNA sequence for each of the endophytes listed in Table 2 was determined and is set forth in SEQ ID NOs: 5-67.

Accordingly, the disclosure provides a bacterial endophyte comprising a 16S rRNA gene comprising a nucleotide sequence that comprises a sequence set forth in SEQ ID NOs: 5-67, or its progeny, or an isolated culture thereof, or mutants thereof. In one embodiment, the bacterial endophyte comprises a sequence set forth in SEQ ID NOs: 5-67 and is from a plant identified in Table 2. For example, in one embodiment the bacterial endophyte is designated <EKM101P>, is a Paenibacillus, has a 16S rRNA sequence comprising the sequence set forth in SEQ ID NO: 5. Optionally, the endophyte is isolated from cucumber. In one embodiment the bacterial endophyte is designated EKM203B, is a Bacillus, has a 16S rRNA sequence comprising the sequence set forth in SEQ ID NO: 6. Optionally, the bacterial endophyte is isolated from Cantaloupe. In one embodiment, the endophyte is designated EKM201D, is a Pediococcus, and has a 16S rRNA sequence comprising the sequence set forth in SEQ ID NO: 60.

The 16S rRNA gene is widely used for the classification and identification of microbes. It is well known in the art that bacteria of the same species need not share 100% sequence identity in the 16S rRNA sequences. Accordingly, in one aspect of the disclosure, the bacterial endophyte has a 16S rRNA gene comprising a nucleotide sequence that has at least 95% sequence identity to a sequence set forth in SEQ ID NOs: 5-67. In another aspect, the bacterial endophyte has a 16S rRNA gene comprising a nucleotide sequence has at least 96%, 97%, 97.5%, 98%, 98.5%, 99%, 99.5%, 99.9% or 100% sequence identity to the sequence set forth in SEQ ID NOs: 5-67.

In another aspect, the bacterial endophyte has a 16S rRNA gene comprising at least 100, 200, 300, 400, 450, 500 or 525 consecutive nucleotides of the sequence set forth in SEQ ID NOs: 5-67.

The bacterial endophytes identified in Table 2 can readily be obtained using the information and methods described herein. For example, the bacterial endophytes can be isolated from the plant sources identified in Table 2. Endophytes can be tested for the characteristics properties and/or anti-pathogenic activities identified in Tables 3-9 by using the in vitro and in planta methods described in the Examples. Furthermore, bacterial endophytes having a 16S rRNA gene with sequence identity to the sequence set forth in SEQ ID NO:5-67 can be identified by sequencing the 16S rRNA gene of a bacterial endophyte and comparing the sequence to SEQ ID NOs: 5-67 to determine the percentage of sequence identity.

In one embodiment, there is provided a bacterial endophyte as described herein that inhibits the growth of powdery mildew. Bacterial endophytes that inhibit the growth of powdery mildew are identified in Tables 7, 8 and/or 9. Bacterial endophytes such as those listed in the Tables may be identified and/or isolated using the characteristics and methods described herein including, but not limited to, plant source, bacterial genus, 16S rDNA sequence, in vitro characteristics including those listed in Tables 4 and 5, as well as in planta in planta activity against fungal pathogens.

In one embodiment, there is provided a bacterial endophyte that has activity against powdery mildew and at least one additional fungal pathogen. For example, in one embodiment, the bacterial endophyte inhibits the growth of powdery mildew and one or more of Fusarium graminearum, Rhizoctonia solani, Phytophthora capsici and Pythium aphanideratum.

In one embodiment, there is provided a bacterial endophyte that has activity against a fungal pathogen and exhibits one or more characteristics selected from RNase activity, production of acetoin and diacetyl, growth on nitrogen-free media, ACC deaminase activity, phosphate solubilisation, auxin production, siderophore production, cellulase activity, pectinase activity and protease activity. For example, in one embodiment there is provided a bacterial endophyte that has activity against powdery mildew and exhibits the characteristic of auxin production. In another embodiment, there is provided a bacterial endophyte that has activity against powdery mildew and one or more of RNase activity, production of acetoin and diacetyl and phosphate solubilisation activity.

In one embodiment, there is provided a bacterial endophyte that inhibits the growth of powdery mildew selected from EKM101V, EKM102Z, EKM103V, EKM201S, EKM301K, EKM201D, EKM505B and EKM419B. In one embodiment, there is provided a bacterial endophyte that inhibits the growth of powdery mildew selected from EKM101V, EKM102Z, EKM103V, EKM201S, EKM301K, EKM201D, EKM505B and EKM419B and exhibits one or more of RNase activity, acetoin & diacetyl production and phosphate solubilisation.

In one embodiment, the endophyte is EKM101V as described herein, its progeny or mutants thereof. In one embodiment, the endophyte EKM101V is of the genus Pantoea and has a 16S rDNA sequence with sequence identity to:

(SEQ ID NO: 43)
GTTCCTTGAGGAGTGGCTTCCGGAGCTAACGCGTTAAGTCGACCGCCTGG
GGAGTACGGCCGCAAGGTTAAAACTCAAATGAATTGACGGGGGCCCGCAC
AAGCGGTGGAGCATGTGGTTTAATTCGATGCAACGCGAAGAACCTTACCT
ACTCTTGACATCCAGAGAACTTTCCAGAGATGGATTGGTGCCTTCGGGAA
CTCTGAGACAGGTGCTGCATGGCTGTCGTCAGCTCGTGTTGTGAAATGTT
GGGTTAAGTCCCGCAACGAGCGCAACCCTTATCCTTTGTTGCCAGCGATT
CGGTCGGGAACTCAAAGGAGACTGCCGGTGATAAACCGGAGGAAGGTGGG
GATGACGTCAAGTCATCATGGCCCTTACGAGTAGGGCTACACACGTGCTA
CAATGGCGCATACAAAGAGAAGCGACCTCGCGAGAGCAAGCGGACCTCAC
AAAGTGCGTCGTAGTCCGGATCGGAGTCTGCAACTCGACTCCGTGAAGTC
GGAATCGCTAGTAATCGTGGATCAGAATGCCACGGTGAATACGTTCCCGG
GCCTTGTACACACCGCCCGTCACACCATGGGAGTGGGTTGCAAAAGAAGT
AGGTAGCTTAACCTTCGGGAGGGCGCTTACCACTTTGTGATTCATGACTG
GGGTGAAGTCGTAACA

In one embodiment, the endophyte has a 16S rDNA sequence with sequence identity to SEQ ID NO: 43 and exhibits the characteristics of producing acetoin & diacetyl, auxin and siderophores. In one embodiment, the endophyte EKM101V is isolated from cucumber seed biogel (early surface sterilization rinse including placenta and fruit pulp tissue).

In another embodiment, the endophyte is EKM102Z as described herein its progeny or mutants thereof. In one embodiment, the endophyte EKM102Z is an Unclassified Enterobacteriaceae and has a 16S rDNA sequence with sequence identity to:

(SEQ ID NO: 33)
TGAGGCGTGGCTTCCGGAGCTAACGCGTTAAGTCGACCGCCTGGGGAGTA
CGGCCGCAAGGTTAAAACTCAAATGAATTGACGGGGGCCCGCACAAGCGG
TGGAGCATGTGGTTTAATTCGATGCAACGCGAAGAACCTTACCTACTCTT
GACATCCAGAGAACTTAGCAGAGATGCTTTGGTGCCTTCGGGAACTCTGA
GACAGGTGCTGCATGGCTGTCGTCAGCTCGTGTTGTGAAATGTTGGGTTA
AGTCCCGCAACGAGCGCAACCCTTATCCTTTGTTGCCAGCGGTTAGGCCG
GGAACTCAAAGGAGACTGCCAGTGATAAACTGGAGGAAGGTGGGGATGAC
GTCAAGTCATCATGGCCCTTACGAGTAGGGCTACACACGTGCTACAATGG
CGCATACAAAGAGAAGCGACCTCGCGAGAGCAAGCGGACCTCATAAAGTG
CGTCGTAGTCCGGATTGGAGTCTGCAACTCGACTCCATGAAGTCGGAATC
GCTAGTAATCGTGGATCAGAATGCCACGGTGAATACGTTCCCGGGCCTTG
TACACACCGCCCGTCACACCATGGGAGTGGGTTGCAAAAGAAGTAGGTAG
CTTAACCTTCGGGAGGGCGCTTACCACTTTGTGATTCATGACTGGGGTGA
AGTCGTAACAAGGTA

In one embodiment, the endophyte has a 16S rDNA sequence with sequence identity to SEQ ID NO: 43 and exhibits the characteristics of producing acetoin and diacetyl, and auxin. In one embodiment, the endophyte EKM102Z is isolated from cucumber seed biogel (early surface sterilization rinse including placenta and fruit pulp tissue).

In another embodiment, the endophyte is EKM103V as described herein, its progeny or mutants thereof. In one embodiment, the endophyte EKM103V is of the genus Pantoea and has a 16S rDNA sequence with identity to:

(SEQ ID NO: 54)
GGGGGCCCGCACAAGCGGTGGAGCATGTGGTTTAATTCGATGCAACGCGA
AGAACCTTACCTACTCTTGACATCCAGAGAACTTTCCAGAGATGGATTGG
TGCCTTCGGGAACTCTGAGACAGGTGCTGCATGGCTGTCGTCAGCTCGTG
TTGTGAAATGTTGGGTTAAGTCCCGCAACGAGCGCAACCCTTATCCTTTG
TTGCCAGCGATTCGGTCGGGAACTCAAAGGAGACTGCCGGTGATAAACCG
GAGGAAGGTGGGGATGACGTCAAGTCATCATGGCCCTTACGAGTAGGGCT
ACACACGTGCTACAATGGCGCATACAAAGAGAAGCGACCTCGCGAGAGCA
AGCGGACCTCACAAAGTGCGTCGTAGTCCGGATCGGAGTCTGCAACTCGA
CTCCGTGAAGTCGGAATCGCTAGTAATCGTGGATCAGAATGCCACGGTGA
ATACGTTCCCGGGCCTTGTACACACCGCCCGTCACACCATGGGAGTGGGT
TGCAAAAGAAGTAGGTAGCTTAACCTTCGGGAGGGCGCTTACCACTTTGT
GATTCATGACTGGGGTGAAGTCGTACAA

In one embodiment, the endophyte has a 16S rDNA sequence with sequence identity to SEQ ID NO: 54 and exhibits the characteristics of producing acetoin & diacetyl, auxin and siderophores. In one embodiment, the endophyte EKM103V is isolated from cucumber seed biogel (early surface sterilization rinse including placenta and fruit pulp tissue).

In another embodiment, the endophyte is EKM201S as described herein, its progeny or mutants thereof. In one embodiment, the endophyte EKM201S is of the genus staphylococcus and has a 16S rDNA sequence with sequence identity to:

(SEQ ID NO: 36)
TAGGGGGTTTCCGCCCCTTAGTGCTGCAGCTAACGCATTAAGCACTCCGC
CTGGGGAGTACGACCGCAAGGTTGAAACTCAAAGGAATTGACGGGGACCC
GCACAAGCGGTGGAGCATGTGGTTTAATTCGAAGCAACGCGAAGAACCTT
ACCAAATCTTGACATCCTCTGACCCCTCTAGAGATAGAGTTTTCCCCTTC
GGGGGACAGAGTGACAGGTGGTGCATGGTTGTCGTCAGCTCGTGTCGTGA
GATGTTGGGTTAAGTCCCGCAACGAGCGCAACCCTTAAGCTTAGTTGCCA
TCATTAAGTTGGGCACTCTAAGTTGACTGCCGGTGACAAACCGGAGGAAG
GTGGGGATGACGTCAAATCATCATGCCCCTTATGATTTGGGCTACACACG
TGCTACAATGGACAATACAAAGGGTAGCGAAACCGCGAGGTCAAGCAAAT
CCCATAAAGTTGTTCTCAGTTCGGATTGTAGTCTGCAACTCGACTACATG
AAGCTGGAATCGCTAGTAATCGTAGATCAGCATGCTACGGTGAATACGTT
CCCGGGTCTTGTACACACCGCCCGTCACACCACGAGAGTTTGTAACACCC
GAAGCCGGTGGAGTAACCTTTTGGAGCTAGCCGTCGAAGGTGGGACAAAT
GATTGGGGTGAAGTCGTAAC

In one embodiment, the endophyte has a 16S rDNA sequence with sequence identity to SEQ ID NO: 36 and exhibits the characteristics of acetoin & diacetyl production. In one embodiment, the endophyte EKM201S is isolated from honeydew melon seeds.

In another embodiment, the endophyte is EKM301K as described herein, its progeny or mutants thereof. In one embodiment, the endophyte EKM301K is an Unclassified Enterobacteriaceae and has a 16S rDNA sequence with sequence identity to:

SEQ ID NO: 47)
TGTTCCTTGAGGAGTGGCTTCCGGAGCTAACGCGTTAAGTCGACCGCCTG
GGGAGTACGGCCGCAAGGTTAAAACTCAAATGAATTGACGGGGGCCCGCA
CAAGCGGTGGAGCATGTGGTTTAATTCGATGCAACGCGAAGAACCTTACC
TACTCTTGACATCCAGAGAACTTAGCAGAGATGCTTTGGTGCCTTCGGGA
ACTCTGAGACAGGTGCTGCATGGCTGTCGTCAGCTCGTGTTGTGAAATGT
TGGGTTAAGTCCCGCAACGAGCGCAACCCTTATCCTTTGTTGCCAGCGAT
TCGGTCGGGAACTCAAAGGAGACTGCCAGTGATAAACTGGAGGAAGGTGG
GGATGACGTCAAGTCATCATGGCCCTTACGAGTAGGGCTACACACGTGCT
ACAATGGCATATACAAAGAGAAGCGACCTCGCGAGAGCAAGCGGACCTCA
TAAAGTATGTCGTAGTCCGGATTGGAGTCTGCAACTCGACTCCATGAAGT
CGGAATCGCTAGTAATCGTGGATCAGAATGCCACGGTGAATACGTTCCCG
GGCCTTGTACACACCGCCCGTCACACCATGGGAGTGGGTTGCAAAAGAAG
TAGGTAGCTTAACCTTCGGGAGGGCGCTTACCACTTTGTGATTCATGACT
GGGGTGAAGT

In one embodiment, the endophyte has a 16S rDNA sequence with sequence identity to SEQ ID NO: 47 and exhibits the characteristics of RNase activity, phosphate solubilisation and auxin production. In one embodiment, the endophyte EKM301K is isolated from pumpkin seeds.

In one embodiment, the endophyte is EKM201D as described herein, its progeny or mutants thereof. In one embodiment, the endophyte EKM201D is of the genus Pediococcus and has a 16S rDNA sequence with sequence identity to:

(SEQ ID NO: 60)
GTGCTGCAGCTAACGCATTAAGTAATCCGCCTGGGGAGTACGACCGCAAG
GTTGAAACTCAAAAGAATTGACGGGGGCCCGCACAAGCGGTGGAGCATGT
GGTTTAATTCGAAGCTACGCGAAGAACCTTACCAGGTCTTGACATCTTCT
GACAGTCTAAGAGATTAGAGGTTCCCTTCGGGGACAGAATGACAGGTGGT
GCATGGTTGTCGTCAGCTCGTGTCGTGAGATGTTGGGTTAAGTCCCGCAA
CGAGCGCAACCCTTATTACTAGTTGCCAGCATTAAGTTGGGCACTCTAGT
GAGACTGCCGGTGACAAACCGGAGGAAGGTGGGGACGACGTCAAATCATC
ATGCCCCTTATGACCTGGGCTACACACGTGCTACAATGGATGGTACAACG
AGTCGCGAGACCGCGAGGTTAAGCTAATCTCTTAAAACCATTCTCAGTTC
GGACTGTAGGCTGCAACTCGCCTACACGAAGTCGGAATCGCTAGTAATCG
CGGATCAGCATGCCGCGGTGAATACGTTCCCGGGCCTTGTACACACCGCC
CGTCACACCATGAGAGTTTGTAACACCCAAAGCCGGTGGGGTAACCTTTT
AGGAGCTAGCCGTCTAAGGTGGGACAGATGATTAGGGTGAA

In one embodiment, the endophyte has a 16S rDNA sequence with sequence identity to SEQ ID NO: 60 and exhibits phosphate solubilisation. In one embodiment, the endophyte EKM201D is isolated from cantaloupe seeds.

In one embodiment, the endophyte is EKM505B as described herein, its progeny or mutants thereof. In one embodiment, the endophyte EKM505B is of the genus Bacillus and has a 16S rDNA sequence with sequence identity to:

(SEQ ID NO: 34)
TAGGGGGTTTCCGCCCCTTAGTGCTGCAGCTAACGCATTAAGCACTCCGC
CTGGGGAGTACGGTCGCAAGACTGAAACTCAAAGGAATTGACGGGGGCCC
GCACAAGCGGTGGAGCATGTGGTTTAATTCGAAGCAACGCGAAGAACCTT
ACCAGGTCTTGACATCCTCTGACAATCCTAGAGATAGGACGTCCCCTTCG
GGGGCAGAGTGACAGGTGGTGCATGGTTGTCGTCAGCTCGTGTCGTGAGA
TGTTGGGTTAAGTCCCGCAACGAGCGCAACCCTTGATCTTAGTTGCCAGC
ATTCAGTTGGGCACTCTAAGGTGACTGCCGGTGACAAACCGGAGGAAGGT
GGGGATGACGTCAAATCATCATGCCCCTTATGACCTGGGCTACACACGTG
CTACAATGGACAGAACAAAGGGCAGCGAAACCGCGAGGTTAAGCCAATCC
CACAAATCTGTTCTCAGTTCGGATCGCAGTCTGCAACTCGACTGCGTGAA
GCTGGAATCGCTAGTAATCGCGGATCAGCATGCCGCGGTGAATACGTTCC
CGGGCCTTGTACACACCGCCCGTCACACCACGAGAGTTTGTAACACCCGA
AGTCGGTGAGGTAACCTTTTAGGAGCCAGCCGCCGAAGGTGGGACAGATG
ATTGGGGTGAAGTCGTAAC

In one embodiment, the endophyte has a 16S rDNA sequence with sequence identity to SEQ ID NO: 34 and exhibits RNase activity, phosphate solubilisation and auxin production. In one embodiment, the endophyte EKM505B is isolated from spaghetti squash seeds.

In one embodiment, the endophyte is EKM419B as described herein, its progeny or mutants thereof. In one embodiment, the endophyte EKM419B is of the genus Bacillus and has a 16S rDNA sequence with sequence identity to:

(SEQ ID NO: 66)
TAGTGTTAGAGGGTTTCCGCCCTTNAGTGCTGCAGCTAACGCATTAAGCA
CTCCGCCTGGGGAGTACGGTCGCAAGACTGAAACTCAAAGGAATTGACGG
GGGCCCGCACAAGCGGTGGAGCATGTGGTTTAATTCGAAGCAACGCGAAG
AACCTTACCAGGTCTTGACATCCTCTGACAACTCTAGAGATAGAGCGTTC
CCCTTCGGGGGACAGAGTGACAGGTGGTGCATGGTTGTCGTCAGCTCGTG
TCGTGAGATGTTGGGTTAAGTCCCGCAACGAGCGCAACCCTTGATCTTAG
TTGCCAGCATTTAGTTGGGCACTCTAAGGTGACTGCCGGTGACAAACCGG
AGGAAGGTGGGGATGACGTCAAATCATCATGCCCCTTATGACCTGGGCTA
CACACGTGCTACAATGGATGGTACAAAGGGCTGCAAGACCGCGAGGTCAA
GCCAATCCCATAAAACCATTCTCAGTTCGGATTGTAGGCTGCAACTCGCC
TACATGAAGCTGGAATCGCTAGTAATCGCGGATCAGCATGCCGCGGTGAA
TACGTTCCCGGGCCTTGTACACACCGCCCGTCACACCACGAGAGTTTGTA
ACACCCGAAGTCGGTGGAGTAACCGTAAGGAGCTAGCCGCCTAAGGTGGG
ACAGATGATTGGGGTGAAGT

In one embodiment, the endophyte has a 16S rDNA sequence with sequence identity to SEQ ID NO: 66 and exhibits the characteristics of RNase activity, auxin production and protease activity. In one embodiment, the endophyte EKM419B is isolated from watermelon seeds.

The bacterial endophytes described herein may be grown in culture medium using methods and compositions known in the art and described herein. For example, the bacterial endophytes may be cultured by shake flask cultivation, small scale or large scale cultivation (including but not limited to continuous, batch, fed-batch, or solid state cultivations) performed in suitable medium and under conditions allowing cell growth. The cultivation may take place in suitable nutrient medium comprising carbon and nitrogen sources and inorganic salts, using procedures known in the art. In one embodiment, the bacterial endophytes are cultured using Lysogeny Broth (LB) or another culture media suitable for the growth a bacterial endophyte.

Compositions for inoculating the plants with the isolated bacterial endophyte described herein are also disclosed. In one aspect, the disclosure provides an inoculating composition, comprising a bacterial endophyte comprising a 16S rRNA gene comprising a nucleotide sequence that has at least 90%, 95%, 96%, 96.5%, 97%, 97.5%, 98%, 98.5%, 99%, 99.5% or 100% sequence identity to a sequence set forth in SEQ ID NOs: 5-67, or its progeny, or an isolated culture thereof or mutants thereof and optionally, a carrier.

As used herein, the term “carrier” refers to the means by which the bacterial endophyte is delivered to the target plant. Carriers that may be used in accordance with the present disclosure include oils, polymers, plastics, wood, gels, colloids, sprays, drenching means, emulsifiable concentrates and so forth. The selection of the carrier and the amount of carrier used in a composition may vary and depends on several factors including the specific use and the preferred mode of application.

The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the invention. Water-in-oil emulsions can also be used to formulate a composition that includes the purified bacterial population. Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products, for example, ground grain or beans, broth or flour derived from grain or beans, starch, sugar, or oil.

In some embodiments, the agricultural carrier may be soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, sugar cane bagasse, hulls or stalks from grain processing, ground plant material or wood from building site refuse, sawdust or small fibers from recycling of paper, fabric, or wood. Other suitable formulations will be known to those skilled in the art.

In one embodiment, the composition comprises a suspension of the isolated bacterial endophyte and a seed coating agent as carrier. Optionally, the seed coating agent is polyvinyl pyrrolidine (PVP). In one example, 500 μL of bacterial suspension, optionally 10 μL to 1 mL of bacterial suspension, is mixed with 10 mL of PVP, optionally 1 mL to 100 mL PVP.

In one embodiment, the composition includes at least one member selected from the group consisting of a tackifier, a microbial stabilizer, a fungicide, an antibacterial agent, an herbicide, a nematicide, an insecticide, a plant growth regulator, a rodenticide, a dessicant, and a nutrient.

In one embodiment, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amic (Helena), Kinetic (Helena), Sylgard 309 (Wilbur-Ellis) and Century (Precision). In one embodiment, the surfactant is present at a concentration of between 0.01% (v/v) to 10% (v/v). In another embodiment, the surfactant is present at a concentration of between 0.1% (v/v) to 1% (v/v).

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some cases, it is advantageous for the formulation to contain agents such as a fungicide, an antibacterial agent, an herbicide, a nematicide, an insecticide, a plant growth regulator, a rodenticide, and/or a nutrient. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In one embodiment of the disclosure, the composition is in a fluid form suitable for spray application or seed coating. In the liquid form, for example, solutions or suspensions, the bacterial endophytic populations of the present invention can be mixed or suspended in water or in aqueous solutions. Suitable liquid diluents or carriers include water, aqueous solutions, petroleum distillates, or other liquid carriers.

In another embodiment, said composition is in a paste-like form. In still another embodiment, the composition is in a substantially dry and powdered form for dusting. Solid compositions can be prepared by dispersing the bacterial endophytic populations of the invention in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The composition is optionally applied as a spray. In another embodiment, the composition is applied to seeds, as a seed coating. In yet another embodiment, the composition is applied both as a spray and a seed coating. In one embodiment, the composition or formulation described herein is a foliar spray. In one embodiment, the methods described herein include inoculating a plant using a foliar spary comprising a bacterial endophyte.

In an embodiment, the composition comprises a suspension of the isolated bacterial endophyte and a seed coating agent as carrier. Optionally, the seed coating agent is polyvinyl pyrrolidine (PVP). In one example, 500 μL of bacterial suspension, optionally 10 μL to 1 mL of bacterial suspension, is mixed with 10 mL of PVP, optionally 1 mL to 100 mL PVP.

In another embodiment, the composition comprises a suspension of the bacterial endophyte and a carrier suitable for spray application. For example, bacteria may be suspended in 10 mM Tris HCl, pH 7 to OD₅₉₅ 0.5, optionally OD₅₉₅ 0.1-0.8.

The formulations comprising the bacterial endophytic population of the present invention typically contains between about 0.1 to 95% by weight, for example, between about 1% and 90%, between about 3% and 75%, between about 5% and 60%, between about 10% and 50% in wet weight of the bacterial population of the present invention. It is preferred that the formulation contains at least about 10³ CFU per mL or gram of formulation, for example, at least about 10⁴ CFU, at least about 10⁵ CFU, at least about 10⁶ CFU, at least 10⁷ CFU, at least about 10⁸ CFU per mL, or at least about 10⁹ CFU per mL of formulation. In one embodiment, the formulation comprises between about 10⁴ CFU per mL and about 10⁹ CFU per mL, between about 10⁵ CFU per mL and about 10⁹ CFU per mL or between about 10⁵ CFU per mL and 10⁸ CFU per mL. In one embodiment, the formulation comprises between about 10⁴ CFU per gram of formulation and about 10⁹ CFU per gram of formulation, between about 10⁵ CFU per gram of formulation and about 10⁹ CFU per gram of formulation or between about 10⁵ CFU per gram of formulation and 10⁸ CFU per gram of formulation.

In one embodiment, the composition can inhibit the growth of a plant pathogen such as a fungus. One of skill in the art can readily determine the amount or concentration of the composition that can be applied to the plant or plant seed to inhibit a particular pathogen such as Fusarium graminearum or powdery mildew. For example, the bacterial density of the inoculate can range from an OD₆₀₀ of 0.1 to 0.8. In one embodiment, the bacterial density of the inoculate at OD₆₀₀ is 0.5, optionally approximately 0.5.

Also provided are synthetic combinations of the isolated bacterial endophyte described herein in association with a plant. In one embodiment, the bacterial endophytes reside within the seeds, roots, stems and/or leaves of the plant as an endophyte. Optionally, the plant may be a plant seed or seedling. In one embodiment, the bacterial endophyte is heterologous to the microbial population of the plant. For example, synthetic combination may be a bacterial strain having antifungal properties as described herein which has been artificially inoculated on a plant that does not naturally harbor or contain the bacterial endophyte.

In one embodiment, the plant is a plant listed in Table 2. In one embodiment, the plant is a curcurbit species. In one embodiment, the plant is plant is an agricultural plant. In one embodiment, the plant is a fruit or vegetable. In one embodiment, the plant is an organism that lives in association with endophytes that reside in the internal tissues of the plant.

In one embodiment, the synthetic combination comprises one or more bacterial endophytes as described herein at concentrations higher than would be found occurring naturally between a cucurbit species and the endopohyte. For example, in one embodiment, the synthetic combination comprises a seed and a bacterial endophyte as described herein at a concentration of at least 10², 10³, 10⁴, 10⁵, 10⁶, 10⁷, 10⁸ or 10⁹ CFU per seed.

In one embodiment, the synthetic combination comprises one or more bacterial endopytes as described herein at a concentration or amount effective to provide a benefit to seeds, seedlings and/or plants derived from the synthetic combination. In one embodiment, the benefit is selected from from the group consisting of improved plant growth, increased plant mass, and increased plant yield. In one embodiment, the benefit is inhibiting the growth of a fungal pathogen such as powdery mildew. In one embodiment, the bacterial endophyte is present in the synthetic combination at a concentration or amount effective to provide a benefit (such as, but not limited to, inhibiting the growth of powdery mildew) in a plant produced from a synthetic combination comprising a seed in association with a bacterial endophyte relative to a plant produced from a seed that is not in association with the bacterial endophyte.

In one embodiment, the synthetic combination comprises a plant such as a fruit or vegetable, optionally a curcubit species. In one embodiment, the plant is an agricultural plant other than a cucurbit species. In one embodiment, the synthetic combination comprises a bacterial endophyte and a plant heterologous to the endophyte, such as a plant other than the plant from which bacterial endophyte was isolated. For example, in one embodiment endophyte is selected from EKM101V, EKM102Z, EKM103V, EKM201S, EKM301K, EKM201D, EKM505B and EKM419B and the plant is a fruit or vegetable other than cucumber, honeydew melon, pumpkin, cantaloupe melon, sphaghetti squash or watermelon.

Optionally, the synthetic combination further comprises one or more of a carrier, tackifier, microbial stabilizer, fungicide, antibacterial agent, herbicide, nematicide, insecticide, plant growth regulator, rodenticide, dessicant and nutrient.

In another embodiment, there is provided an article of manufacture or kit that includes packaging material, plant seeds within the packaging material, and at least one species of bacterial endophyte associated with the seeds. In one embodiment, the article of manufacture comprises a synthetic combination as described herein. In one embodiment, at least 100, 200, 500, 750, 1000, 1250, 1500, 1750, 2000, 3000, 4000 or 5000 seed weight amount of seeds are disposed within the packaging material. Optionally, a carrier may also be disposed within the article of manufacture in association with the seeds.

### III. Methods and Uses of the Bacterial Endophytes

In one aspect, it has been shown that the bacterial endophytes described herein exhibit one or more characteristics that are beneficial for plants associated with the bacterial endophytes. For example, as shown in Examples 1 and 2, the bacterial endophytes identified in Table 2 may confer beneficial properties such as improving the growth or yield of a plant. The bacterial endophytes identified in Table 2 may also be used as biological control agents to inhibit or prevent the growth of plant pathogens.

Therefore, in one aspect, the disclosure provides a method of preventing or inhibiting growth of a pathogen on a plant, comprising inoculating the plant with one or more of the bacterial endophytes described herein. In one embodiment, the disclosure provides a method of preventing or inhibiting infection of a plant by a pathogen, comprising inoculating the plant with one or more of the bacterial endophytes described herein.

In another embodiment, the disclosure provides a use of the bacterial endophytes described herein to prevent or inhibit fungal growth on a plant.

“Inhibiting growth of a pathogen” includes, but is not limited to, decreasing the amount of pathogen on a plant, decreasing the speed of pathogen growth and/or infection on a plant, decreasing the severity of an infection on a plant, decreasing the amount of diseased area of a plant, decreasing the percentage or number of infected seeds, decreasing the percentage or number of disease lesions on a plant decreasing the percentage of apparent infection of a plant and/or treating or preventing growth of a pathogen in a plant. In one embodiment, the pathogen is a fungus. On one embodiment, the pathogen is selected from Fusarium graminearum, Rhizoctonia solani, Phytophthora capsici and Pythium aphanideratum. In one embodiment, the pathogen is powdery mildew such as Podosphaera fuliginea or Podosphaera xanthii. Any of the above criteria can be decreased by at least 5%, 7.5%, 10%, 15%, 20%, 25%, 30%, 40%, 50%, 75%, 100%, 150% or 200% in an inoculated plant compared to a non-inoculated plant.

“Inhibiting growth of a pathogen” also includes preventing infection by a pathogen.

“Inhibiting growth of a pathogen on a plant” can result in improved growth of the inoculated plant. Determining an improvement in plant growth using the bacterial endophytes described herein can be assessed in a number of ways. For example, the size or weight of the entire plant or a part thereof (such as seeds or roots) can be measured. In an embodiment, the average mass of an inoculated plant is increased at least 5%, 7.5%, 10%, 15%, 20%, 25%, 30%, 40%, 50%, 75%, 100%, 150% or 200% fresh weight or dry weight. In another embodiment, the average mass of the seeds of an inoculated plant is increased at least 5%, 7.5%, 10%, 15%, 20%, 25%, 30%, 40%, 50%, 75%, 100%, 150% or 200% fresh weight or dry weight. In still another embodiment, the endophyte-associated plant exhibits at least 4%, 5%, 6%, 7%, 8%, 9%, 10%, 15%, 20%, 25%, 30%, 40%, 50%, 75%, 100%, 150% or 200% or more fruit or grain yield, than the reference agricultural plant grown under the same conditions.

The bacterial endophytes described herein may also be used as prophylactic agents to decrease the chance of an infection by a pathogen from occurring in a plant. For example, in one embodiment, the endophytes may be used to decrease the chance of infection by a fungus or powdery mildew. In one embodiment, the endophytes may be used to decrease the chance of infection by Fusarium graminearum, Rhizoctonia solani, Phytophthora capsici and/or Pythium aphanideratum.

In one embodiment, the bacterial endophytes, methods and uses therefore described herein may be used to control or prevent infection by powdery mildew. In one embodiment, the powdery mildew is caused Podosphaera fulginea (powdery mildew). In another embodiment, the pathogen is a genera and species in the order Erysiphales underlying powdery mildew infection in a host plant. For example, in one embodiment, the pathogen is Podosphaera leucotricha and causes powdery mildew in apples and/or pears. In one embodiment, the pathogen is Podosphaera aphanis and causes powdery mildew in strawberries. In one embodiment, the pathogen is Erysiphe necator and causes powdery mildew in grapes. In one embodiment, the pathogen is Blumeria graminis and causes powdery mildew in wheat.

The methods described herein can be applied to any plant in need thereof. It is known that bacterial endophytes readily colonize a wide diversity of plant species and thus inoculation with the endophytes described herein will colonize a variety of plant species. In one embodiment, the plant is an agricultural plant. In one embodiment, the plant is a fruit or vegetable. In one embodiment, the plant is a cucurbit species.

In one embodiment, the plant is a grape. In one embodiment, the plant is a small grain, such as but not limited to wheat or pea plants. In one embodiment, the plant is a tree fruit, such as but not limited to apples. In one embodiment, the plant is a leafy vegetable, such as but not limited to arugula or parsley. In one embodiment, the plant is an ornamental, such as but not limited to roses.

The plant can be inoculated with the bacterial endophytes described herein or a composition comprising the bacterial endophytes described herein, using techniques known in the art. For example, the bacterial endophytes may be applied to the roots of the plant, or to young germinated seedlings, or to ungerminated or germinated seeds. In one embodiment, the bacterial endophytes may be sprayed onto the plant, either in a liquid formulation or as a powdered inoculum. Alternatively, a composition comprising the bacterial endophytes can be applied by drip or other irrigation system.

In one embodiment, the methods described herein may be used to inoculate a plant in order to provide more than one benefit to the plant. As demonstrated in the Examples, it has surprisingly been determined that some bacterial endophytes inhibit the growth or more than one fungal pathogen. In one embodiment the methods described herein are therefore useful for inhibiting the growth of two or more fungal pathogens selected from powdery mildew, Fusarium graminearum, Rhizoctonia solani, Phytophthora capsici and Pythium aphanideratum.

In one embodiment, the bacterial endophytes, methods and uses therefore described herein may be used for preparing an agricultural seed composition. For example, in one embodiment there is provided a method comprising contacting a plurality of seeds with a formulation comprising a bacterial endophyte identified in one or more of Tables 2-9. In one embodoiment, the bacterial endophyte is identified in one or more of Tables 7-9 and the method is useful for preparing an agricultural seed composition that is resistant to the growth of a fungal pathogen such as powdery mildew.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present application:

### EXAMPLES

### Example 1: Isolation and Characterization of Cultured Seed Associated Microbes of the Cucurbit Family

### SUMMARY

Endophytes are microbes that colonize plant internal tissues without causing disease. In particular, seed-associated endophytes may be vectors for founder microbes that establish the plant microbiome, which may subsequently contribute beneficial functions to their host plants including nutrient acquisition and promotion of plant growth. The Cucurbitaceae family of gourds (e.g. cucumbers, melons, pumpkin, squash), including its fruits and seeds, is widely consumed by humans. However, there is limited data concerning the taxonomy and functions of seed-associated endophytes across the Cucurbitaceae family. Here, bacteria from surface-sterilized seeds of seven economically important cucurbit species were cultured, classified using 16S rRNA sequencing, and subjected to eight in vitro functional tests.

In total, 169 unique seed-associated bacterial strains were cultured from selected cucurbit seeds. Interestingly, nearly all strains belonged to only two phyla (Firmicutes, Proteobacteria) and only one class within each phyla (Bacilli, γ-proteobacteria, respectively). Bacillus constituted 50% of all strains and spanned all tested cucurbit species. Paenibacillus was the next most common genus, while strains of Enterobacteriaceae and lactic acid bacteria were also cultured. Phylogenetic trees showed limited taxonomic clustering of strains by host species. Surprisingly, 33% of strains produced the plant hormone, indole-3-acetic acid (auxin), known to stimulate the growth of fruits/gourds and nutrient-acquiring roots. The next most common nutrient acquisition traits in vitro were (in rank order): nitrogen fixation/N-scavenging, phosphate solubilisation, siderophore secretion, and production of ACC deaminase. Secretion of extracellular enzymes required for nutrient acquisition, endophyte colonization and/or community establishment were observed. Bacillus strains had the potential to contribute all tested functional traits to their hosts.

The seeds of economically important cucurbits tested in this study have a culturable core microbiota consisting of Bacillus species with potential to contribute diverse nutrient acquisition and growth promotion activities to their hosts. These microbes may be useful as seed inoculants to assist sustainable food production. Given that cucurbit seeds are consumed by traditional societies as a source of tryptophan, the precursor for auxin, it is possible that human selection inadvertently facilitated auxin-mediated increases in gourd size.

### Methods

### Sources of Cucurbit Seeds

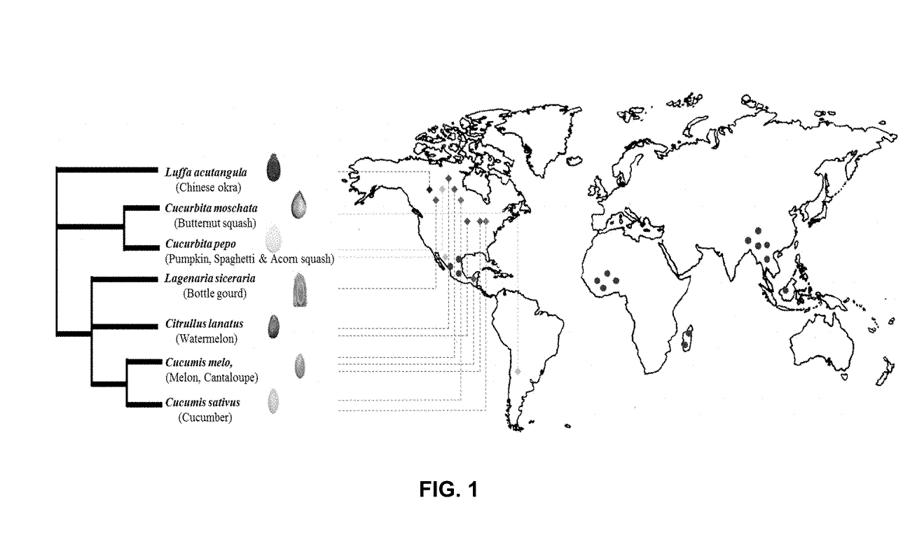

The seeds of 21 cucurbit varieties, representing seven species and five genera, were used in this study as sources of endophytes (Table 1, FIG. 1); these represent a relatively diverse phylogeny of economically important cucurbit species [30], [31]. Commercial dry seeds and fresh fruits were purchased from different seed companies and local grocery stores in Ontario, Canada, respectively (Table 1). Fresh melons and squash were imported from different countries in North, Central and South America.

### Surface Sterilization of Commercial Seeds

Seeds were surface sterilized using modifications of a frequently used protocol, adjusted for the varying texture of cucurbit seeds [8]. Per variety, three replicate pools of seeds were surface sterilized. For each replicate, 15 seeds per variety were surface sterilized using sodium hypochlorite (2.5-3.5%) for 5 min (8 min for Jack O'Lantern pumpkin seeds) based on differences in the texture of the seed coat, then the bleach was drained. This step was performed twice, and then the seeds were rinsed with autoclaved distilled water, before being washed with 95% ethanol for 5 min. After draining the ethanol, the seeds were rinsed three times for 5 min with autoclaved distilled water. To verify that the surface sterilization was adequate, 200 μL of the last wash were plated and cultured on three different types of agar media: LGI agar to capture contaminating diazotrophic bacteria [25 g sucrose, 0.01 g FeCl₃.6H₂O, 0.8 g K₃PO₄, 0.2 g MgSO₄.7H₂O, 0.2 g CaCl₂, 0.002 g Na₂MoO₄.2H₂O, agar 15 g/L, pH 7.5], potato dextrose agar (PDA) (#70139, Sigma) to culture copiotrophic bacteria and fungi, and (Reasoner's 2A agar) R2A (#17209, Sigma) to capture oligotrophic bacteria. Plates were incubated at room temperature for three days.

### Extraction of Fresh Seeds

Three fruits were used for each variety representing three replicate pools of seeds. The extrasion of fresh seeds was was started by washing the fruits with soap and water, then the fruits were heavily sprayed with 70% ethanol in the laminar flow cabinet and left to dry. Fifteen fresh seeds were extracted from each fruit under aseptic conditions using sterile and/or autoclaved tools. The seeds were rinsed with autoclaved distilled water for seven to ten times (based on differences in the fibres and flesh coating of the seeds) till get rid of the surrounding fibers and mucilage. 200 μL of last washes were cultured on the same agar media used for verification of surface sterilization of commercial seeds to ensure null microbial growth.

### Isolation of Bacterial Endophytes

Once seed surface sterilization was confirmed, 15 seeds/replicate were ground gently in an autoclaved mortar using 0.5 mL of 50 mM Na₂HPO₄ buffer per gram of seed dry weight [29]. The ground seed suspension (100 μL) was used for microbial culturing: 10-fold serial dilutions in 50 mM Na₂HPO₄ (10×, 100× and 1000×) were streaked on R2A, PDA and LGI agar plates (described above) followed by incubation for 1-7 days at 28° C. Morphologically unique bacterial colonies from each plate were selected, streaked on fresh plates to purify and finally cultured in LB broth (10 g/L tryptone, 5 g/L yeast extract and 5 g/L NaCl, pH 7.2) for glycerol stocks and DNA isolation.

Bacterial Endophyte 16S rRNA Genes Fingerprinting

For identifying bacterial endophytes, DNA was isolated using Qiagen kits (#51306, QIAamp® DNA Mini kit) and then quantified (Nanodrop, Thermo Scientific, USA). PCR was performed using 16S rRNA genes universal primer pair [32], 799F (5′-AACMGGATTAGATACCCKG-3′) (SEQ ID NO: 1) and 1492r (5′-GGTTACCTTGTTACGACTT-3′) (SEQ ID NO: 2). Approximately 50 ng of total DNA was added to a PCR mixture containing 4 μL Standard Taq Buffer (M791B, Promega), 0.4 μL of 25 mM dNTP mix, 0.5 μL of each primer (10 mM working stock), 0.6 μL of 50 mM MgCl₂, 0.2 μL of Standard Taq (New England Biolabs) and H₂O to a final volume of 20 μL. Following an initial denaturation at 94° C. for 7 min, DNA was amplified using 34 PCR cycles in a PTC200 DNA Thermal Cycler (MJ Scientific, USA) at 94° C. for 45 sec, 48° C. for 1 min, 72° C. for 2 min, and a final extension at 72° C. for 7 min. Amplicons of 693 bp size were gel purified using the GFX™ PCR DNA and Gel Band purification kit (#45001489, GE Healthcare). Purified amplicons were sequenced using standard BigDye reaction conditions with an annealing temperature of 50° C. (3730 DNA analyzer, Applied Biosystems, USA). Reads were searched against bacterial 16S entries in RDP (Ribosomal Database Project [33] using a 95% confidence level, and further searched using BLASTN using default parameters. To generate maximum likelihood (ML) phylogenetic trees with bootstrapping of 500 replicates, all 16S sequences were trimmed and edited using Bioedit software [34], then aligned and used to generate the trees using MEGA6 software [35].

### Characterization of Bacterial Endophytes

The endophytes were subjected to eight in vitro tests. All experiments were performed in triplicate. Bacterial endophytes were initially preserved as glycerol stocks in 96-well plates with adjusted OD₆₀₀≈0.4-0.6. Prior to each in vitro test, 96-well LB broth plates (1 mL/well) were inoculated from glycerol stock plates and incubated overnight at 37° C. with shaking. These cultures were then used to inoculate the media described below using a flame-sterilized 96 pin replicator:

### Growth on Nitrogen Free LGI Media

All bacterial isolates were tested for their ability to fix and/or scavenge nitrogen [29], [36]. Prior to media preparation, all glassware was cleaned with 6 M HCl. An autoclaved 96 deep-well plate (2 mL well volume) was pipetted with 1 mL/well of sterile LGI broth [25 g sucrose, 0.01 g FeCl₃.6H₂O, 0.8 g K₃PO₄, 0.2 g MgSO₄.7H₂O, 0.2 g CaCl₂ and 0.002 g Na₂MoO₄.2H₂O, pH 7.5]. Following bacterial inoculation with the pin replicator, the plate was sealed with a sterile breathable membrane, incubated at 28° C. with gentle shaking, and OD₆₀₀ readings were taken after 5 days.

### 1-Aminocyclopropane-1-Carboxylate (ACC) Deaminase Activity

Bacterial endophytes were screened for growth in the presence of ACC as their only source of nitrogen [29]. All glassware was cleaned with 6 M HCl before media preparation. 2 M ACC (#A3903, Sigma) was prepared in water and filter sterilized. Autoclaved LGI broth was amended with ACC solution to a final concentration of 1 μL/mL, and distributed in a new 96 deep-well plate (as above). Following bacterial inoculation with the pin replicator, breathable membranes were used to seal plates, and then the plates were incubated at 28° C. with gentle shaking. After 5 days, OD₆₀₀ readings were taken, and only wells with a significant increase in OD than their corresponding nitrogen-free LGI wells were scored as ACC deaminase producers.

### Mineral Phosphate Solubilization

For detecting phosphate solubilizing microbes, Pikovskaya's agar [37] was inoculated using the pin replicator. The media was prepared as follows: 0.5 g/L yeast extract, 10 g/L glucose, 5 g/l Ca₃PO₄, 0.5 g/L (NH4)₂SO₄, 0.2 g/L, KCl, 0.1 g/L MgSO₄, 0.0001 g/L MnSO₄, 0.0001 g/L FeSO4.7H₂O, and 15 g/L agar, pH 7.2, then autoclaved (glucose was first filter sterilized and added after autoclaving). After incubation for 3 days at 28° C. in darkness, clear halos around colonies indicated the ability of microbes to solubilize tricalcium phosphate.

### Production of Auxin (Indole-3-Acetic Acid, IAA)

Using the 96 pin replicator, bacterial endophytes were plated on autoclaved LB agar (10 g/L tryptone, yeast extract 5 g/L, 5 g/L NaCl and 12 g/L agar, pH 7.2) supplemented with L-tryptophan to a final concentration of 5 mM [38]. The plates were incubated at 28° C. for 3 days, then overlaid with a pure nitrocellullose membrane, and kept overnight in the fridge at 4° C., to allow the permeation of bacteria and their metabolites into the membrane. The next day, the nitrocellulose membrane was transferred to Whatman #2 filter papers impregnated in Salkowski reagent [2% 0.5 M FeCl₃ in 35% perchloric acid (#311421, Sigma)] and left for 30 min. Dark pink halos around colonies in the membrane were interpreted as evidence for auxin production.

### Siderophore Production

All glassware was deferrated with 6 M HCl and distilled, deionized water prior to media preparation [39]. In this deferrated glassware, LB agar media was prepared, autoclaved, poured into 150 mm Petri dishes and inoculated with bacteria using the 96 pin replicator. Plates were overlaid with O-CAS overlay after a 3 day incubation period at 28° C. [40]. For preparing 1 litre of O-CAS overlay, in deferrated glassware, a mixture consisting of 30.24 g of finely crushed piperazine-1,4-bis-2-ethanesulfonic acid (PIPES) with 10 mL of 1 mM FeCl₃.6H₂O in 10 mM HCl solvent, was added slowly with stirring to a mixture of 60.5 mg of chromeazurol S (CAS) and 72.9 mg of hexadecyltrimethyl ammonium bromide (HDTMA). Pre-warmed O-CAS was trapped in melted 1% agarose in a proportion of 3:1 (v/v) just prior to pouring the overlay. The reaction was allowed to incubate for 15 min at room temperature, and then color changes were recorded by scoring purple halos as catechol-type siderophores and orange colonies as hydroxamate-type siderophores.

### Cellulase Activity

To test the cellulase activity of bacterial isolates, R2A media was supplemented with 0.2% (w/v) carboxymethylcellulose (CMC) sodium salt (C-8758, Sigma) and 0.1% Triton X-100 then autoclaved and poured into 150 mm plates [41]. Bacterial endophytes were inoculated using the 96 pin plate replicator, and then the plates were incubated in darkness at 28° C. for 3 days.

Bacterial isolates displaying cellulase activity were visualized by flooding the plate with Gram's iodine. Colonies surrounded by clear halos were interpreted as positive results.

### Pectinase Activity

A previously published method was adapted [42] as described earlier [29]. R2A media amended with 0.2% (w/v) of citrus pectin (P9135, Sigma) and 0.1% Triton X-100 was autoclaved and poured in 150 mm Petri plates. Following bacterial inoculation with the 96 well pin replicator, the plate was incubated at 28° C. for 3 days. The plate was then flooded with Gram's iodine. Pectinase activity was scored as development of clear halos around colonies.

### Protease Activity

Plates of tryptic soy agar/20 (twenty-fold diluted tryptic soy broth and 15 g/L agar) amended with 5% (v/v) skimmed milk were inoculated using the 96 pin plate replicator into 150 mm Petri plates [43], [44]. The plates were incubated at 28° C. for 1-2 days, and positive colonies were scored as surrounded by clear halos.

### Results

### Taxonomic Diversity of Cultivated Cucurbit Seed-Associated Microbiota

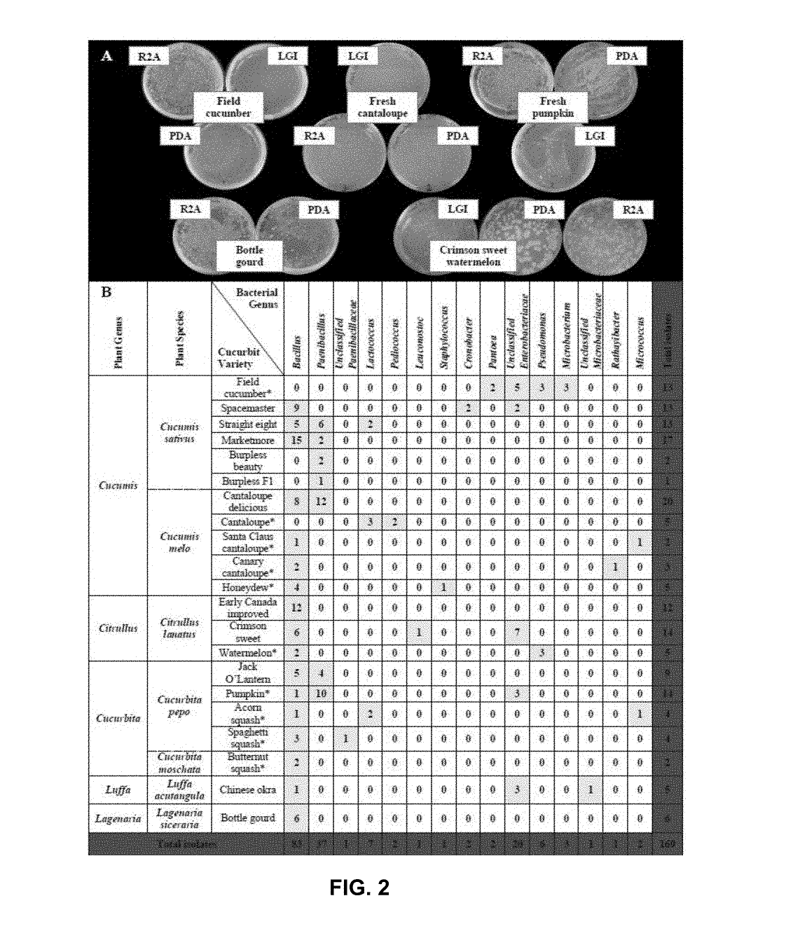

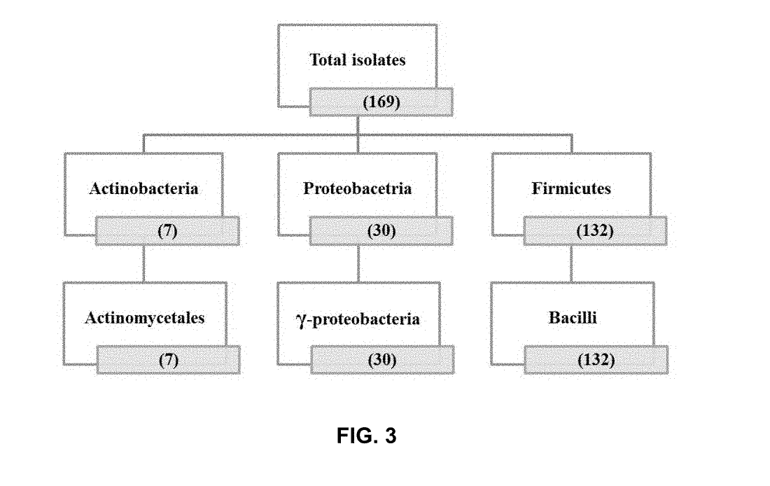

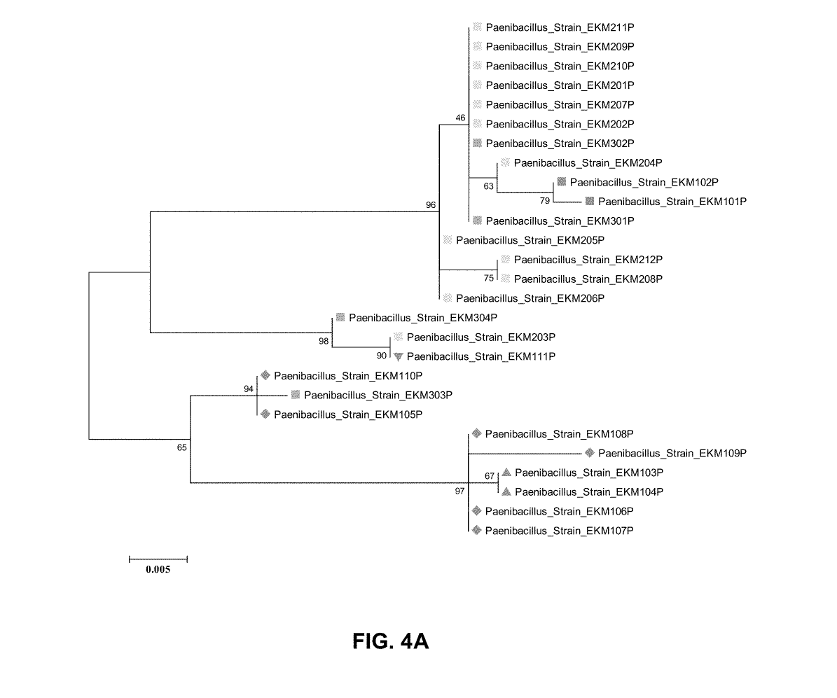

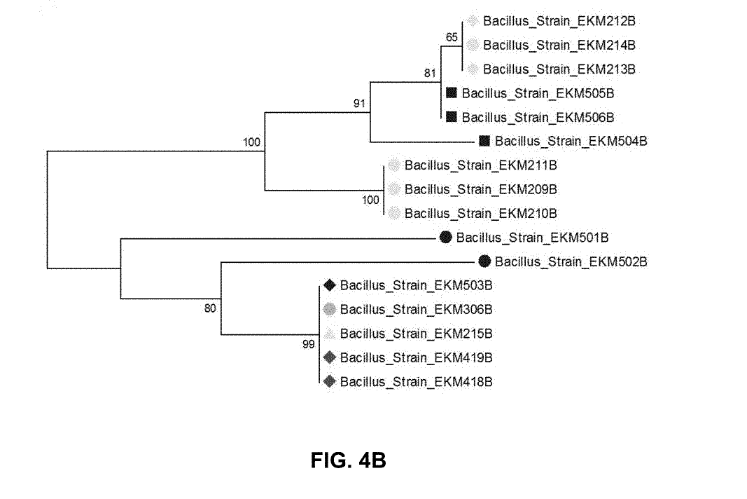

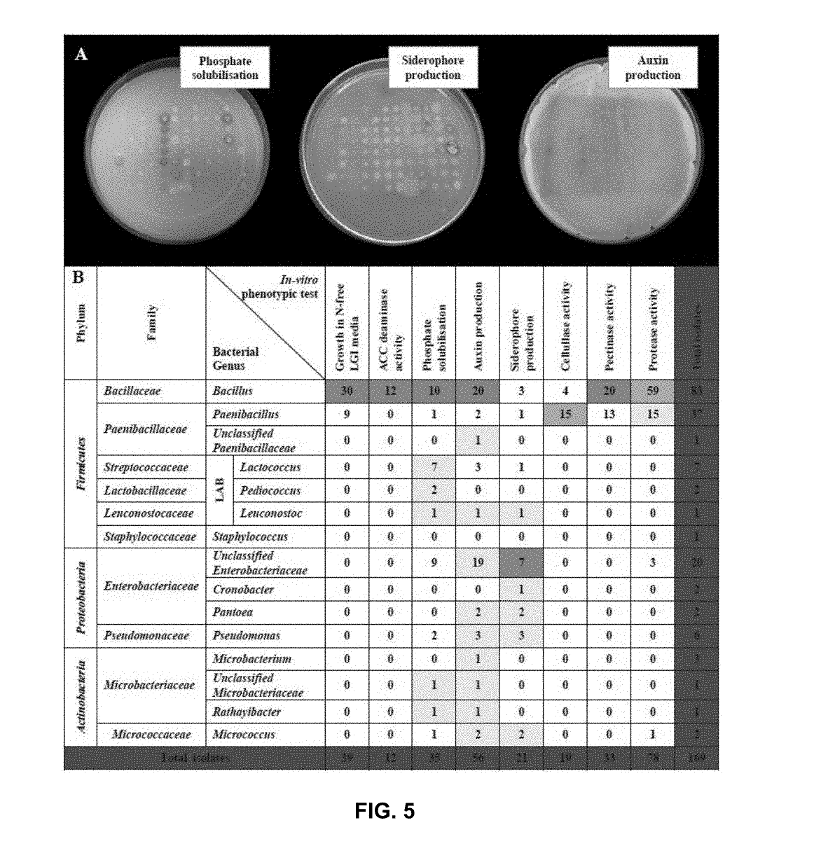

From the tested curcurbit species and varieties (Table 1), a total of 169 unique seed-associated bacterial strains were cultured (FIG. 2). For taxonomic identification, V5-V9 regions of 16S rRNA genes were amplified (ranging in length from 334-682 bp) and searched using BLASTN and RDP databases. A total of 134 strains showed 100% nucleotide sequence identity to the RDP database, 12 showed 95% identity and 23 showed <95% identity at a confidence level of 95%. Best sequence matches from RDP classifier were used to create Maximum likelihood tree(s), a summary of which is shown in FIG. 3. ML phylogenetic trees for were generated for 16S rRNA sequences of bacterial endophytes isolated from dry commercial seeds and fresh fruit seeds (data not shown). A ML phylogenetic tree of 16S rRNA sequences of total bacterial endophytes isolated from both sources was also generated (data not shown). These sequences were deposited in Genbank and received the following accession numbers: [GenBank:KT220264, KT220265], [GenBank:KT222780-KT222785], [GenBank:KT281286-KT281446]. 16S rRNA sequences are also identified herein for specific endophytes in Table 2 as SEQ ID NOs: 5-67. The phylogenetic composition of cultivated cucurbit seed-microbiota showed two significant findings: First, the cultured strains were predicted to belong to 15 bacterial genera within three phyla (Firmicutes, Proteobacteria and Actinobacteria) and within each phyla, all microbes belonged to one class/order only (class Bacilli, class γ-proteobacteria and order Actinomycetales, respectively) (see e.g. FIG. 3). Second, the Bacilli class predominated across the five tested cucurbit genera, belonging to six different families (Bacillaceae, Paenibacillaceae, Streptococcaceae, Lactobacillaceae, Leuconostocaceae and Staphylococcaceae) (see e.g. FIG. 3, FIG. 4, and FIG. 5B). Less common were two families of γ-proteobacteria (Enterobacteriaceae, Pseudomonaceae) and two families of Actinobacteria (Microbacteriaceae and Micrococcoceae).

### Conservation of Cucurbit Seed-Associated Microbiota

The genus Bacillus was the cultured core seed-associated microbiota, spanning the five tested cucurbit genera and accounting for ˜50% (83/169) of the total isolates (FIG. 2B), of which 80 strains showed 100% sequence identity to Bacillus 16S rRNA genes in RDP, while the remaining 3 strains showed 99% identity. The strains were predicted to predominantly belong to diverse Bacillus species based on 16S rRNA genes BLASTN searches to Genbank such as B. safensis, B. altitudinis, B. siamensis, B. pumilus and B. cereus. Strains of the genus Paenibacillus were the next most common (37/169), exclusively isolated from Cucumis sativus L., Cucumis melo L. and Cucurbita pepo L. var pepo L. Although members of Enterobacteriaceae family were less abundant (24/169), they showed higher taxonomic diversity on BLASTN searches (FIG. 2B), though the 16S rRNA genes sequence identities were often low. Interestingly, three different genera of lactic acid bacteria (LAB) were recovered (10/169) from C. sativus L., C. melo L., Citrullus lanatus var. lanatus and Cucurbita pepo L. var. turbinata (FIG. 2B).

### Clustering of Bacillus and Paenabacillus Communities by Host

Using 16S rRNA genes sequences, some clustering of microbes by host were observed for both commercial and fresh seeds. Strains belonging to the two most abundant genera, Bacillus and Paenabacillus, were then phylogenetically classified using maximum likelihood analysis method, again separated for commercial and fresh seed sources (see exemplary dendrograms in FIG. 4). With respect to commercial varieties, the Bacillus strains isolated from cucumber generally clustered separately from melon; pumpkin and bottle gourd Bacillus strains co-clustered with cucumber. Bacillus strains isolated from commercial watermelon showed three clusters, a large group (containing two subgroups) that clustered with melon, and two smaller groups that clustered with cucumber. With respect to Paenabacillus, strains from commercial cucumber generally separated from commercial melon, while pumpkin strains were interspersed. Most pumpkin Paenabacillus strains from this study were from fresh seeds and are shown on a separate tree). Some microbial clusters spanned different varieties within a host species, and hence were not variety-specific.

### Host Specific Microbiota

It was a striking feature that only a single bacterial genus from the seeds of some cucurbit varieties was amenable to being cultured (FIG. 2B). For instance, from fresh field cucumber seeds, only Microbacterium strains could be isolated; however other bacterial isolates were cultured from an early surface sterilization rinse that included residual placenta and fruit pulp tissues. Similarly, 100% of strains cultured from seeds of Early Canada improved watermelon, butternut squash and bottle gourd were predicted to be Bacillus. By contrast, all isolates cultured from fresh cantaloupe were predicted to be lactic acid bacteria comprising two bacterial genera, Lactococcus and Pediococcus (FIG. 2B). On the other hand, the isolated bacterial endophytes from cucumber (Cucumis sativus L.) and angled luffa (Luffa acutangula) spanned the three phyla (Firmicutes, Proteobacteria and Actinobacteria). However, the identified isolates from other tested cucurbit species were restricted to one or two phyla (ML analysis of 16S rRNA gene sequences). For example, endophytic bacteria from watermelon (Citrullus lanatus) and pumpkin (C. pepo L. var. pepo L.) belong to the phyla Firmicutes and Proteobacteria. Nevertheless, all bacterial isolates from bottle gourd exclusively belong to Firmucutes.

### Functional Diversity of Cultivated Cucurbit Seed-Associated Mcrobiota Grouped by Bacterial Genus

Bacterial endophytes were phenotypically tested in vitro for traits associated with plant nutrient acquisition (nitrogen, phosphorus, iron, proteases to scavenge amino acids), and plant growth promotion (ACC deaminase activity, auxin production) along with traits associated with bacterial colonization and community establishment (cellulase, pectinase, proteases) (FIG. 5). The most prevalent phenotypic traits were protease activity, auxin production, nitrogen fixation/N-scavenging (growth on LGI media) and phosphate solubilization, representing ˜46%, ˜33%, ˜23% and ˜21% of all isolates, respectively. The rarest trait was ACC deaminase (7% of strains). Of the strains that might contribute N-fixation/N-scavenging activity to their hosts, 30/39 were from Bacillus. With respect to auxin producing strains, 20/56 were Bacillus, while 21/56 were from Enterobacteriaceae. Of the strains that may contribute ACC deaminase activity, 12/12 were from Bacillus. For siderophore activity, 10/21 strains were from Enterobacteriaceae. Most or all of the strains that showed protease, pectinase or cellulase activities belonged to Bacillus and Paenibacillus. Phosphate solubilization was more randomly distributed, though 10/10 of the lactic acid producing strains showed this activity. It is noteworthy, that combined, the Bacillus strains could contribute all of the traits to their hosts, and furthermore that 21/24 Enterobacteriaceae strains were auxin producers (FIG. 5B).

### Functional Diversity of Cultivated Cucurbit Seed-Associated Microbiota Grouped by Their Host Plants

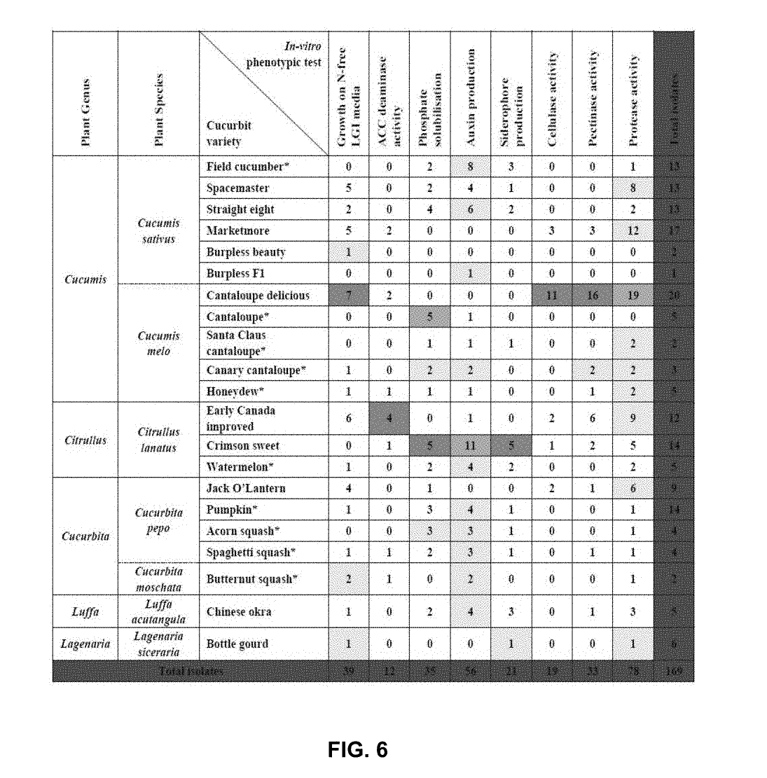

All endophytic phenotypic traits spanned all tested cucurbit species with a few exceptions for Luffa acutangula and Lagenaria siceraria but these were only represented by a single variety each (FIG. 6). For example, of the 56 strains that produced auxin, 19 were isolated from cucumber, 5 from melon, 16 from watermelon, 12 from pumpkin/squash, and 4 from luffa. A few varieties were inhabited by microbes that may have had biased phenotypic activities; for example, most of the seed-associated microbiota from Cantaloupe delicious produced extracellular enzymes. Of the 14 isolates from Crimson sweet watermelon, 11 were auxin producers. Of the 10 lactic acid bacteria (mineral phosphate solubilizers), 5 were isolated from fresh cantaloupe.

### DISCUSSION

Seeds are critical vectors for diverse pathogenic and phyto-beneficial microbes including endophytes. There are three main routes of microbial transmission from mother to seeds: via the internal vascular system, foliar transmission through stigma, or direct contact with microbial communities on adjacent maternal organs such as fruits and flowers [45]. Physiological changes during seed maturation influence the functional properties of seed-colonizers and their taxonomic diversity. Following germination, the seed-associated endophytes have the potential to become the founders of the seedling microbiome, to provide beneficial functions to the host such as nutrient acquisition activities within the spermosphere and rhizosphere [4].

The Cucurbitaceae is one of the most economically important families of crops, used as food and for industrial and medical purposes [22], [46]. Up to 50% of the weight of cucurbit seeds consists of oil, and another 35% of protein, making them a richer source of nutrients than most cereals and legumes [47], [48] which has contributed to them being staple food sources in traditional societies [49], [50]. The cucurbits were domesticated on diverse continents, and there is tremendous genetic variation within the family (Table 1) [23]. The inventors hypothesized that the most critical cucurbit endophytes would be transmitted by seeds and would be conserved across the family, reflecting an ancient evolutionary origin, predating species diversification. From this study, a total of 169 seed-associated bacterial endophytes were cultured from 21 varieties representing seven different species of nutritious cucurbits. Despite this diversity, the results demonstrate that economically important cucurbit species possess a core culturable, seed-associated microbiota, dominated by the genus Bacillus (FIG. 2). Based on in vitro testing, this study further demonstrates that tested cucurbit seeds are potential vectors for microbes with diverse nutrient acquisition activities (FIG. 5 and FIG. 6). These microbes were isolated primarily from internal seed tissues, and a limited number from the seed surface (fresh field cucumber).

### Phyla and Class Level Diversity of Seed Associated Endophytes Across the Tested Cucurbits.

This study represented seven species and 21 varieties of cucurbit seeds including dried seeds from diverse commercial sources, and fresh seeds from plants grown in diverse agro-ecological environments (Table 1). Despite this diversity, the putative endophytes belonged to only 15 bacterial genera within three phyla, with 7 genera from Firmicutes, ≧4 from Proteobacteria, and 4 from Actinobacteria on RDP searches (FIG. 2). These results are consistent with previous reports [4], [13], with a recent meta-analysis reporting that the majority of seed endophytes from diverse plant species belong to the phylum Proteobacteria (represented by 80 genera), especially γ-proteobacteria (41 genera), followed by Actinobacteria (25 genera) and Firmicutes (20 genera) [4]. The present disclosure further shows that within each of the phyla (Firmicutes, Proteobacteria, Actinobacteria), the seed-associated endophytes of the cucurbits belong to one class/order only (class Bacilli, class γ-proteobacteria and order Actinomycetales, respectively) (FIG. 3).

### Conservation and Clustering of Bacillus and Paenibacillus Across the Tested Cucurbits

A striking feature of this study is that the genus Bacillus appears to comprise the culturable core seed microbiota across the five tested cucurbit genera. Paenibacillus was the next dominant genus, but culturable from only three of the cucurbit species (FIG. 2B). Together, these two genera comprised 120/169 of the total isolated endophytes. Only limited clustering of Bacillus and Paenibacillus strains by host species and variety was observed (see e.g. FIG. 4), which may suggest that the functional traits of the microbes may be as important as their taxonomy (see below). Both Bacillus and Paenibacillus are gram-positive, aerobic, endospore-forming bacteria, that inhabit different ecological niches such as plants, rhizosphere, soil and water [51]. Previous studies from other plants have shown that a characteristic feature of seed endophytes is that they are endospore forming; this property is thought to protect the seed inhabitants from changes within the seed (to tolerate storage, desiccation, seed maturation, germination) [4]. Bacillus strains, in particular, are important commercial biofertilizers and/or biopesticides used for crop production [52], [53], [54].

Both Bacillus and Pseudomonas have been shown to be the most dominant bacterial genera of the seeds of other plants, in addition to other genera such as Paenibacillus, Micrococcus, Staphylococcus, Pantoea, and Actinobacter [4]. Though Bacillus and Paenibacillus were common in the tested cucurbit seeds, these genera are rare in maize (Zea) seeds [29], suggesting that the cucurbits may have selected these genera for specific ecological purposes. There are few relevant reports concerning cucurbit seed endophytes; however an early report from 1976 implied the isolation of Bacillus strains [55]. More recent research aimed at developing a synergistic biocontrol strategy for pumpkin diseases noted the isolation of anti-pathogenic strains of Paenibacillus polymyxa from pumpkin seeds, Lysobacter gummosus and Pseudomonas chlororaphis from roots and Serratia plymuthica from flowers [25], [26]. Interestingly, the application of fluorescence in situ hybridization for the discovery of bacterial endophytes residing the seed cavity of selected cucurbit species revealed the ability of α, β, γ-proteobacteria, Firmicutes and Actinobacteria to live inside the fruit and the predominance of the genus Bacillus across the tested family fruits [5].

In the present study, ˜38% (14/37) of Paenibacillus isolates from the tested cucurbits were associated with pumpkin seeds of which two strains were predicted to be P. polymyxa. The authors of an earlier pumpkin study also examined the microbiota of roots, flowers and fruits, and suggested the prevalence of two bacterial genera, Pseudomonas and Bacillus, that spanned all tested microenvironments, though the study focused on the anti-pathogenic properties of these strains and not biofertilizer activity [25]. Bacillus strains have previously been isolated from different tissues of watermelon (B. amyloliquefaciens, Bacillus subtilis, Bacillus sp.) [56], [57] and Hami melon [58], from the rhizosphere and surface sterilized roots of cucumber [59], [60], and from the leaf surface and rhizosphere of different cucurbits [61]. Future investigations may study whether these root, shoot, rhizosphere associated Bacilli originate from seeds after germination.

As noted above, Bacillus is a commonly used biopesticide [53], [54], [62] which might raise concerns that the strains isolated in this study are commercial inoculants; however, in the present study design, cucurbits were deliberately selected that were not organic certified, with the exception of the cucumber variety Marketmore which was verified by the associated company as having not been treated with inoculants. Furthermore, a large diversity of seed-associated Bacillus isolates was observed (13 different Bacillus species based on BLASTN searches) from varieties grown in different geographic locations (Table 1), but it cannot be ruled out that some of these may have originated from spores deposited in the soil from commercial inoculants.

### Conservation and Diversity of Potential Functional Traits Provided by Cucurbit Seed-Associated Microbiota

Several potential functional traits were conserved in the seed-associated microbiota across the tested cucurbits:

### Auxin Production

In plants, the hormone auxin, specifically indole-3-acetic acid (IAA), is responsible for cell elongation, division and differentiation [63]. Auxin is also synthesized by bacteria, including Pseudomonas, Enterobacter, Pantoea, Acinetobacter, Klebsiella, Bacillus, Agrobacterium, Azotobacter, Micrococcus as well as Paenibacillus polymyxa [51], [64], [65], [66]. From this study, a striking feature was that 33% of all seed-associated bacteria were auxin producers, including 21/24 strains of Enterobacteriaceae, 20/83 Bacillus strains, 3/6 Pseudomonas strains, and two Paenibacillus strains, including one putative P. polymyxa strain (FIG. 5). This result contrasts sharply with seeds from the Zea family, which includes corn, in which only 7% of strains were auxin producers [29]. It is interesting to speculate whether humans inadvertently selected for auxin-producing microbes in cucurbit seeds, as they selected for larger fruits/gourds or more nutritional seeds. In tomato fruit, which is a model system for understanding the genetic regulation of fruit size, the locular cells that surround seeds undergo cell expansion which is associated with larger fruit size; examination of the tomato transcriptome suggested the role of auxin in this cell expansion [67]. Nutritionally, the amino acid tryptophan which is essential for humans, is the precursor for auxin (IAA) synthesis in plants [13]. In pre-Columbian North America, indigenous tribes grew the “three sister” crops, corn, beans and squash; squash is a source for tryptophan which is deficient in corn and low in beans [68], [69]. Squash was first domesticated in western Mexico around 10,000 years ago, followed by maize and then beans [70], [71]. Numerous studies note the value of cucurbit seeds, especially squash, pumpkin and watermelon, as sources of essential amino acids including tryptophan [69], [72], [73], [74], [75], [76]. In this study, ˜70% (39/56) of the auxin producers were indeed isolated from these three cucurbits (FIG. 6). It may be that as humans selected for more nutritional cucurbit seeds containing tryptophan, they facilitated microbial synthesis of auxin, which in turn contributed to increased gourd size—another favoured trait.

Results from testing the bacterial endophytes identified in Table 2 for growth on nitrogen-free (N-free) media, ACC deaminase, phosphate solubilization auxin production, siderophore production, cellulase activity, pectinase activity and protease activity are shown in Table 3 and Table 5.

### ACC Deaminase

Ethylene was originally isolated as the plant hormone that stimulates fruit-ripening. It is also an important stress hormone for plants to signal the onset of abiotic and biotic stress [77]. The enzyme ACC deaminase is secreted by diverse microbes and prevents synthesis of ethylene by converting ACC, the precursor of ethylene, into a-ketobutyrate and ammonia [13], [78]. In the present study, ACC deaminase activity was observed in only 7% of strains, exclusively belonging to the genus Bacillus (FIG. 5), in contrast to previous studies [78] which showed that other genera (including Burkholderia, Enterobacter, and Pseudomonas) produce this enzyme, including 20% of microbes associated with Zea seeds [29]. Plant growth promoting microbes with ACC deaminase activity are thought to assist plants by relieving the inhibitory effect of ethylene on root growth [14].

Nitrogen Fixation and/or Scavenging

Plants form symbiotic relationships with microbes, including endophytes to facilitate biological nitrogen fixation (BNF), the conversion of atmospheric nitrogen gas into a usable form of nitrogen [13], [51]. In this study, 23% of seed associated microbes could grow on nitrogen-free media (FIG. 5), suggestive of either BNF or N-scavenging; all of these strains were Bacillus or Paenibacillus. Diverse species of Bacillus and Paenibacillus have previously reported as nitrogen fixers [79], [80], [81]. These microbes may be tested in planta to verify whether they contribute to improved nitrogen use efficiency in these crops.

### Phosphate Solubilization

Phosphorous is the second most limiting nutrient after nitrogen for plant growth. Despite its abundance in the soil, it is scarcely bioavailable to plants due to its low solubility [82]. Many soil microbes have the ability to solubilize mineral phosphorous through production of organic acids or phosphatases [13]. This functional property was displayed by 21% of strains in this study (FIG. 5) primarily Bacillus, lactic acid bacteria and Enterobacteriaceae, consistent with previous reports [29], [83], [84]. These strains may have potential for future crop improvement.

### Siderophore Production

Like phosphorous, iron primarily exists in insoluble forms, which decreases its bioavailability to plants. Plants and microbes have the ability to overcome this problem by either lowering soil pH through organic acid production or by synthesis of siderophores, which chelate iron [13]. With respect to the present disclosure, only 12% of strains showed evidence of siderophore secretion, of which half belonged to the Enterobacteriaceae, along with three Pseudomonas strains (FIG. 6). These results agree with previous studies reporting the capability of soil-associated fluorescent pseudomonads to secrete iron-chelating compounds such as pyoverdine [85], as well as the secretion of a catecholate-type siderophore called enterobactin which is specific to the Enterobateriacae family [86].

### Extracellular Enzymes

Some extracellular proteases help microbes to colonize plant roots [9], while some proteases secreted by certain strains of Bacillus are toxic to nematodes through cuticle degradation activity [87]. Cellulase and pectinase enzymes have similarly been shown to help degrade plant cell walls to permit entry of root-colonizing endophytes [4]. Extracellular cellulases and proteases are also used by microbes to help construct polysaccharide- and peptide-rich biofilms that help to establish the microbial community and permit attachment to host cells (e.g. rhizosphere surface, inside plants) [16]. In the present disclosure, Bacillus and Paenibacillus accounted for all or most stains that produced extracellular enzymes (protease, cellulase, pectinase) (FIG. 5). In particular, the most common trait of the seed associated microbiota was protease activity (46%, 78/169 isolates), almost all of which were produced by Bacillus (59/83 of isolates) and Paenibacillus (15/37 of isolates). Proteins are a fundamental source of nitrogen in the soil, assimilated by plants through two major mechanisms: first, through root-secreted proteases, and second, by acquisition of intact proteins via endocytosis followed by protease-mediated degradation [88]. In a previous study by Johnston-Monje and Raizada [29], migration of maize seed associated bacterial endophytes to the roots and rhizosphere was observed. It is interesting to hypothesize whether the cucurbits have selected Bacillus microbes as part of their core seed microbiota to assist with protease-mediated nitrogen uptake and assimilation, following their migration to roots.

### Metagenomic Analysis Versus Culture Dependent Methods

Given the microbiome is counted as an extension of the host genome, a limitation of this study was the culture-independent or in situ study of cucurbit seed microbiome which would reveal much insight into the diversity of the seed endophytic communities that failed to grow on the used nutrient agar media. In addition, metagenomics, metatranscriptomics and metaproteomics can help in understanding the functionality of the gene pool of the seed microbiome and the putative interaction between the host and its colonizing microbiome [2]. The high-throughput sequencing technologies are the most recently used approaches that result in massive data reach to terabases of sequences of the variable regions of 16S rRNA genes for prokaryotes and internal transcribed spacer (ITS) for eukaryotic microbes [2], [3]. In a culture-independent study on rice root-associated endophytic microbiome, their beneficial functional traits comprising plant growth promotion and antagonism to pathogens could be predicted by metagenomic analysis of protein domains involved in metabolic processes [89]. Despite the holistic overview that could be interpreted about the microbiome, but metagenomic studies are still missing the more focused phenotypic and molecular studies on individual isolates that help in understanding the detailed mechanisms underlying their characteristics. In addition, the capability of actual testing of isolated endophytes in vivo and the development of novel inoculants. It will be interesting to compare both types of analysis to demonstrate the huge diversity of seed-associated endophytic communities and profoundly understand the plant-microbe interaction leading to the formulation of new inoculants that help in crop growth and protection.

### A Link Between the Cucurbit Seed Microbiome and the Human Gut Microbiome?

The human gut microbiome is affected significantly by diet [90], [91]. Aside from consumption of the gourds (fruits), the seeds of some cucurbits are staple foods in diverse societies, eaten both cooked and raw (e.g. cucumbers) [92]. As seeds are well known to be colonized by microbes [4], [29], an unexplored idea is whether their raw consumption may affect the human gut microbiome [90]. The human gut microbiome is dominated primarily by two phyla (Firmicutes and Bacteroidetes), along with less represented phyla Actinobacteria, Proteobacteria and Verrucomicrobia (constituting 2% of gut microbiota) [90], [91]; the Firmicutes, Actinobacteria and Proteobacteria similarly dominate the cucurbit seed microbiota. Remarkably, the endophytic bacteria identified from cucumber varieties constitute ≈35% of the total isolates and belong to the three phyla. The Maximum Likelihood (ML) tree of the total collection of putative seed-associated endophytes cultured from cucurbits, based on bacterial 16S rRNA genes sequences (data not shown) confirmed that bacterial isolates identified from cucumber seeds span the three identified phyla, and within each phyla, the microbes belong to one class/order only (Firmicutes (class Bacilli), Actinobacteria (order Actinomycetales) and Proteobacteria (γ-proteobacteria); furthermore, many species of these isolates are well-known as probiotics. Based on these results, it is interesting to speculate whether the human gut microbiome is directly or indirectly, influenced by the cultured microbiome of edilble cucurbit seeds, in particular lactic acid bacteria and Bacilli [93], [94].

### CONCLUSIONS

This study is believed to be the first to examine endophytes across diverse cucurbit species and varieties and has revealed a cultured core microbiota associated with the seeds of the most economically important cucurbits, consisting primarily of spore-forming Bacilli. Bacillus is the main isolated bacterial genus with potential to contribute diverse nutrient acquisition and growth promotion activities to their hosts. The seeds may be vectors for these microbes, to help their host plants acquire nutrients and stimulate growth. The endophytes described herein may therefore be useful as novel biofertilizers, coated onto seeds as stable spores. As set out in Example 2, some of the endophytes described herein also have the ability to antagonize cucurbit pathogens.

### Example 2: Characterization and Anti-Pathogenic Activity of Bacterial Endophytes

### Source of Bacterial Endophytes

As set out in Example 1, one hundred and sixty nine bacterial endophytes were cultured and molecularly identified based on 16S rDNA from seeds of diverse cucurbits. The isolation procedure was performed after seeds surface sterilization.

### In Vitro Testing of Functional Traits of Impact on Host Resistance

Acetoin and diacetyl production. The protocol was adapted from the protocol described in reference [95]. Autoclaved LB broth was amended with filter sterilized 0.5% glucose and distributed as 1 mL aliquots in 96 deep well plate (#07-200-700, Fisher). The plates were inoculated with overnight grown bacterial endophytes using a flame-sterilized 96 pin replicator, sealed with a breathable membrane and incubated in shaking incubator (150 rpm at 28° C.) for 5 days. On the fifth day, 100 μL of each bacterial culture was transferred to a 96 well white fluorometer plate using multichannel pipet. 100 μL/well of freshly prepared Barritt's Reagents A and B was added, and then stand for 15 min. Development of red/pink color was scored as positive result against the copper color of the reagent as a negative control. Barritt's Reagents A and B were prepared in a proportion 3:1 (v/v) by mixing 0.5% (w/v) creatine solution with freshly prepared 7.5% (w/v) a-naphthol in 2.5 N sodium hydroxide.

RNAse activity was deteremined as described in reference [96]. In 10 mL of 0.1 M Na₂HPO₄ at pH 8, 1.5 g of torula yeast RNA (#R6625, Sigma) was dissolved, filter sterilized and added to 250 mL of autoclaved LB agar media. Bacterial endophytes were inoculated using flame sterilized pin replicator, and incubated at 28° C. After 3 days, 70% perchloric acid was flooded over the plates for 15 min. Clear halos around colonies were scored as positive results.

Results of screening the bacterial endophytes listed in Table 2 for Acetoin and diacetyl production and RNAse activity are found in Table 4 and Table 6.

### Antifungal Screening of Endophytic Bacteria

### i) Agar Disc Diffusion Technique

The antifungal spectrum of cucurbit seed associated microbiota was tested against a group of phytopathogens with a diverse host ranges including cucurbits. These pathogens were isolated from different crops and vegetable crops and supplied from different plant pathology laboratories. Rhizoctonia solani strain was isolated from sugar beet cultivated in Ontario and identified phenotypically both in plant pathology lab (University of Guelph, Ridge town campus) and in Dr. Linda Hanson at United States Department of Agriculture (USDA) in Michigan. Phytophthora capsici was isolated from pepper grown in Simcoe Station, Ontario and identified phenotypically by Dr. Catarina Saude. Pythium aphanidermatum was obtained from cucumber grown in Quebec, identified and supplied from Mary Ruth research laboratory. Fusarium graminearum was isolated from corn and supplied by Agriculture and Agri-food Canada (AAFC). Using agar diffusion technique, fungal pathogens were grown in YPD broth for 3-5 days at 25° C. with gentle shaking. Pre-melted cooled PDA media was inoculated with the mycelia (25 mL/150 mL PDA for R. solani, 1 mL/50 mL PDA for P. capsici and F. graminearum and 1 mL/100 mL PDA for Pythium aphanidermatum) and poured into sterile Petri dishes (150 mm×15 mm) then left to solidify. Using sterile glass tubes, 11 mm cups were made in the inoculated agar plates. Cups were loaded with 100 μL overnight bacterial enophyte cultures with adjusted OD₆₀₀ 0.4-0.6. The fungicide Nystatin (N 581, PhytoTechnology Laboratories, USA) was dissolved in dimethyl sulfoxide (DMSO) and used as a positive control (≈202 U/100 μl for R. solani, 454 U/100 μl for F. graminearum and 1817 U/100 μl for P. capsici), and DMSO was used as the negative control. Propamocarb hydrochloride 722 g/L (aqueous solution) (UVP:05933765, Bayer CropScience Inc., Canada) was used as a positive control for P. aphanidermatum at its commercial concentration and autoclaved ddH₂O was used as the negative control. The plates were incubated at 25° C. for 3-5 days in darkness. The antifungal activity was scored by measuring the diameter of inhibition zones around cups. The assay was performed in triplicates for each bacterial endophyte.

Results from testing the bacterial endophytes identified in Table 2 against the group of phytopathogens set out above are shown in Table 4 and Table 6.

### ii) In Planta Techniques for Obligate Pathogen

### Plant Variety

National pickling cucumber variety was purchased from William Dam Seeds (LTD), Dundus, Ontario. Seeds were germinated, transplanted and grown in a growth room at 25/18° C. for 16/8 h (day/night), respectively. The humidity was maintained at 75% during the experiment, and the light intensity (cool white fluorescent bulbs) was measured (150-220 μmol m² sec⁻¹) at the top of the plants using a photon flux meter in the photosynthetically active radiation (PAR) range of 400-700 nm (model BQM-01, Apogee Instruments Inc., Logan, Utah, USA).

### Source and Maintenance of Pathogen

Infected cucumber leaves with powdery mildew fungus were obtained from commercial greenhouse in Leamington in Ontario through greenhouse vegetable IPM specialist in Ontario Ministry of Agriculture, Food and Rural Affairs (OMAFRA). The causative pathogen was maintained on healthy cucumber plants (National pickling cucumber variety) plants by tapping infected leaves over new healthy ones in a growth chamber at 24° C./18° C. (day/night), 16/8 h photoperiod (day/night), 60/80% humidity (day/night), under fluorescent light (200 μmol m-2 sec-1).

### Molecular Identification of Powdery Mildew Causative Pathogen

A heavily infected leaf was picked and the spores were collected by spraying with autoclaved 0.01% Triton in ddH₂O. The spore suspension was centrifuged and washed using the same solution at 10,000 rpm for 10 min. Fungal DNA was isolated from the pelleted spores using (#69106, DNeasy® Plant Mini kit) (Qiagen, USA) and quantified by (Nanodrop, Thermo Scientific, USA). PCR was performed using primer pair ITS4 (forward): 5′-TCCTCCGCTTATTGATATGC-3′ (SEQ ID NO:3) and ITS1F (reverse): 5′-CTTGGTCATTTAGAGGAAGTAA-3′ (SEQ ID NO:4) [97]. Approximately 50 ng of total DNA was added to a PCR mixture containing 4 μL Standard Taq Buffer (M791B, promega), 0.4 μL of 25 mM dNTP mix, 0.5 μL of each primer (10 mM working stock), 0.6 μL of 50 mM MgCl₂, 0.2 μL of Standard Taq (New England Biolabs) and H₂O to a final volume of 20 μL. A PCR was undertaken using the following conditions: an initial denaturation at 95° C. for 10 min, PCR was cycled 34 times at 95° C. for 1 min, 51° C. for 1 min, 72° C. for 1 min, and a final extension at 72° C. for 7 min. Amplicons of 600 bp size were gel purified and sequenced as above. Reads were RDP (Ribosomal database project) searched against fungal ITS deposits. The causative pathogen was identified as Podosphaera fuliginea.

### Detached Leaf Disc Bioassay

Adapting previous protocol [98], the leaves from the 2^{nd} to 5^{th} leaves of cucumber plants were detached and 0.5 inch leaf-discs were made by using a paper puncher. Fifteen leaf-discs were used for each endophytic bacteria (three petri-dishes, 5 discs for each) by soaking them in bacterial suspension of Luria Bertani broth amended with filter sterilized 0.01% Tween 20 and adjusted to OD₆₀₀≈0.3-0.5 for 1 minute with gentle shaking. Then five discs were transferred to each petri dish (6 cm in diameter) using flame sterilized forceps where they were placed on a damp filter paper (5.5 cm filter paper Whatman No. 1) wetted with 2 mL of a specific nutrient solution to cucumber plant (for 1 liter of tap water: 950 mg Ca(NO₃)₂. 4H₂O, 810 mg KNO₃; 500 mg MgSO₄.7H₂O; 155 mg NH₄H₂PO₄; 3 mg H₃BO₃; 2 mg; ZnSO4.7H₂O; 0.05 mg CuSO₄.5H₂O; 0.02 mg NaMoO₄; and 25 mg NaFeEDTA [99]. The leaf discs were placed with their adaxial side upside. The petri dishes were incubated at 30° C. for 24 h. Next day and after 24 h, 10 μL of freshly prepared conidial suspension with adjusted concentration 1×10⁵ CFU/mL using a hemocytometer was placed in the center of each leaf disc as a pathogen inoculum. The conidial suspension was prepared by adapting a previous protocol [100] in autoclaved ddH₂O supplemented with 0.01% Triton-X-100. Then petri dishes were sealed with a breathable tape and incubated in growth chamber using the same conditions for pathogen maintenance. Prothioconazole as a chemical fungicide and Bacillus subtilis QST 713 strain as a biofungicide were used as positive controls, and LB broth supplemented with filter sterilized 0.01% Tween 20 was used as a negative control. The evaluation of the disease severity was performed using 0-7 scales based on the development of infected lesions and chlorosis. Disease index and preventive effect were calculated by the following equations:

Disease index (%)=[Σ(Number of disease leaves at each scale×the corresponding scale)/(Total number of investigated leaves×the highest scale)]×100

Preventive effect (%)=[(Disease index of negative control−disease index of treatment)/(Disease index of negative control)×100]

### Detached Whole Leaf Bioassay

To confirm and narrow down promising endophyte candidates from the leaf disc assay prior to greenhouse trials, whole cucumber leaves from the 2^{nd}-5^{th} node were picked [98]. Each leaf was placed on a damp filter paper containing nutrient solution and placed in a 150 mm Petri dish (for 1 L tap water: 950 mg Ca(NO₃)₂. 4H₂O, 810 mg KNO₃; 500 mg MgSO₄.7H₂O; 155 mg NH₄H₂PO₄; 3 mg H₃BO₃; 2 mg; ZnSO₄.7H₂O; 0.05 mg CuSO₄.5H₂O; 0.02 mg NaMoO₄; and 25 mg NaFeEDTA). The petiole of each leaf was wrapped in an autoclaved and wetted cotton puff. Overnight endophyte cultures were harvested and re-suspended in LB broth supplemented with 0.01% Tween 20 and adjusted to OD₆₀₀≈0.3-0.5. The leaves were sprayed with ≈5 mL bacterial suspension using hand sprayers. Petri-dishes were incubated at 30° C. in darkness. After 24 h, the leaves were sprayed with ≈0.5 mL conidial suspension (1×10⁵ CFU/mL) using a hand sprayer. The Petri dishes were sealed with a breathable tape and incubated in a growth chamber for 7 days (under the same conditions used in the leaf disc assay). Positive and negative controls were used exactly as in leaf disc bioassay and each treatment was represented in quintuplicate. The evaluation of the disease severity was performed using the same 0-7 scale based on the development of infected lesions and chlorosis. The disease index and preventive effects were calculated using the same equations used in leaf disc bioassay.

### RESULTS

Results from testing bacterial endophytes against powdery mildew using the detached leaf disc bioassay and detached whole leaf bioassay are shown in Tables 7-9.

Table 7 provides a summary of the identification and characterization of a selected list of bacterial endophytes with activity against powdery mildew. The bacterial endophytes listed in Table 7 demonstrated anti-powdery mildew (Podosphaera fuliginea) activity greater than or equal to that of chemical fungicide prothioconazole in leaf disc bioassay.

### Leaf Disc Bioassay for Antagonism Against Cucumber Powdery Mildew

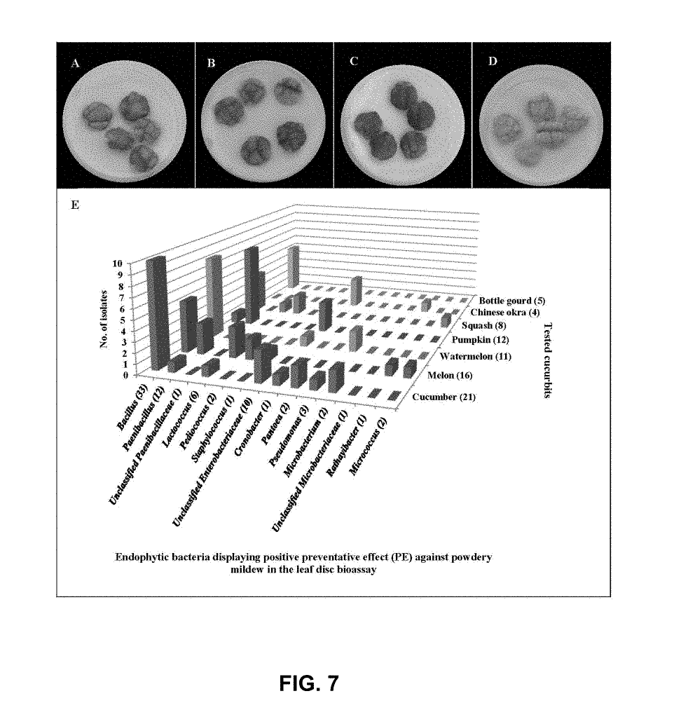

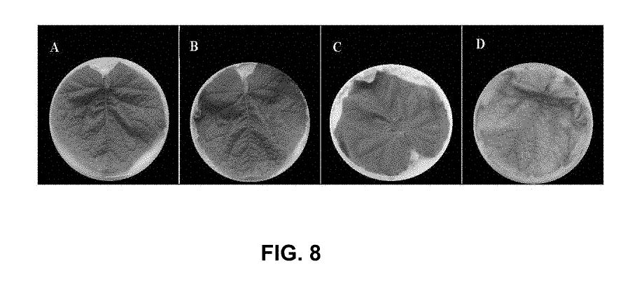

Endophytes were tested for antagonism against obligate pathogen Podosphaera fuliginea, the causative agent of cucumber powdery mildew (FIG. 7A-D and Table 8). The disease index (DI %) and preventive effect (PE %) were calculated for all tested endophytes (Table 7 and Table 8). Remarkably, 46% (77/169) of the tested endophytic bacteria showed in planta antagonism against powdery mildew symptoms in comparison to discs treated with LB broth (negative control) (FIG. 7E). Interestingly, most of the remaining endophytes displayed adverse effects on cucumber leaf health in the presence of the pathogen (data not shown). The beneficial endophytes displayed mean positive PE values of up to 89% by which 53% of strains (41/77) were equally or more effective than the fungicide, prothioconazole (positive control) (Table 8). The concentration of fungicide had been optimized in a pre-experiment (Prothioconazole used in 24 μg/ml concentration and prepared from 480 g/L stock solution and as a commercial biocontrol agent formulated in 1 mg/ml suspension of water dispersible granules) as positive controls. Furthermore, 64% of endophyte strains that showed in plant antagonism against powdery mildew (49/77) were equally or more effective than the commercial biocontrol agent, Serenade™ (Bacillus subtilis QST 713) (Table 8). The endophytes that were equally or more effective than either positive control (total of 49 strains) originated from the five tested cucurbit genera (FIG. 8E and Table 8). Unlike for in vitro antimicrobial activity (shown in FIG. 6), fresh cucurbit seeds were the major sources (69%, 34/49) of powdery mildew in planta antagonists (Table 8). Furthermore, cucumber, pumpkin and melon seeds contributed many of the antagonists, accounting for 22% (11/49), 20% (10/49) an 18% (9/49) of the effective endophytes, respectively. With respect to endophytic taxonomy, the strains that were equally or more effective antagonists than either positive control belonged to 12 different bacterial genera of which the most prevalent (in rank order) were Bacillus (33%, 16/49), Unclassified Enterobacteriaceae (16%, 8/49), and classified Paenibacillus (14%, 7/49). However, amongst the most effective antagonists were Pediococcus and Pantoea endophytes.

### Detached Whole Leaf Bioassay for Antagonism Against Cucumber Powdery Mildew

To independently validate the leaf disc results and to make the assay more robust, the endophytes that displayed activity against powdery mildew in comparison to prothioconazole fungicide (41 strains, Table 7 and Table 8) were re-tested using detached whole cucumber leaves infected with the pathogen (FIG. 8A-D and Table 9). In total, 66% (27/41) of endophytes were confirmed to inhibit powdery mildew symptoms visually in comparison to leaves treated with LB broth (negative control), with PE values ranging from 8%-46% (Table 9). In contrast, the majority of the remaining endophytes displayed adverse effects on cucumber leaf health in the presence of the pathogen. The fresh seeds were the major sources of beneficial microbes, accounting for 89% (24/27) of disease antagonists (Table 9). Unlike the leaf disc bioassay, pumpkin seeds were the main source of promising antagonists accounting for 30% (8/27) of candidates, followed by cucumber seeds 22% (6/27), melon (19%, 5/27) and watermelon seeds (7%, 2/27). Bacillus endophytes were the most prevalent antagonists, comprising 26% (7/27) of the positive strains. However, a Pediococcus endophyte from fresh cantaloupe seeds displayed the highest PE% against the disease (46%), equivalent to the positive controls (Table 9).

### Discussion

The importance of in vitro antimicrobial screening as an initial step in planning a biocontrol strategy is highlighted in the present disclosure. A wide range of host phytopathogens were tested resulting in the identification of a number of bacterial endophytes with robust anti-pathogenic activities. These bacterial endophytes were isolated from a variety of human-domesticated cucurbit crop host plants and belong to various bacterial genus. The taxonomic and functional discovery of plant endophytic microbiome can significantly help in identifying and using endophytes for the promotion of plant growth and assuring protection againt ecological stresses. The use of functional traits as a guide for nomination of biocontrol candidates alongside with molecular identification of bacterial genus as shown in the present disclosure allows for rational development of strategies for future biological control applications of promising endophytic bacteria as antagonists against plant pathogens.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, sequence data accession numbers, patents, and patent applications are herein incorporated by reference in their entirety to the same extent as if each individual publication, sequence data accession number, patent or patent application was specifically and individually indicated to be incorporated by reference in its entirety.

TABLE 1
Summary of cucurbit seeds used as sources of endophytes.

| | No. of diploid | Common | Commercial | | Common |
|---|---|---|---|---|---|
| Taxonomy¹ | chromosomes | name | variety | Source | use |
| Tribe Benincaseae | | | | | |
| Genus Cucumis | | | | | |
| Cucumis melo L. | 24 | Melon | Cantaloupe | Mckenzie, MB, | Food |
| | | | Delicious | Canada, #101114 | |
| | | | Cantaloupe* | Honduras | |
| | | | Santa Claus | USA | |
| | | | cantaloupe* | | |
| | | | Canary | USA | |
| | | | cantaloupe* | | |
| | | | Honeydew* | USA | |
| Cucumis sativus L. | 14 | Cucumber | Field cucumber* | Canada | Food |
| | | | Straight eight | Mckenzie, MB, | |
| | | | Marketmore | Canada, #101250 | |
| | | | (Certified | Mckenzie, MB, | |
| | | | organic seeds) | Canada, #134129 | |
| | | | Spacemaster | Mckenzie, MB, | |
| | | | | Canada, #132403 | |
| | | | Burpless | Burpee, PA, USA | |
| | | | beauty | #52175A | |
| | | | Burpless F1 | Mr. Fothergill's, | |
| | | | | AB, Canada, | |
| | | | | #13194 | |
| Genus Citrullus | | | | | |
| Citrullus lanatus | 22 | Watermelon | Early Canada | Mckenzie, MB, | Food |
| (Thunb). | | | improved | Canada, #101734 | |
| Matsum. &Nakai | | | | | |
| | | | Crimson sweet | Burpee, PA, USA, | |
| | | | | #62679A | |
| | | | Watermelon* | Canada | |
| Genus Lagenaria | | | | | |
| Lagenaria siceraria | 22 | Bottle gourd | Bottle gourd | Ontario Seed Co., | Ornamental |
| (Molina) Stand. | | | | ON, Canada | |
| Tribe Sicyoeae | | | | | |
| Genus Luffa | | | | | |
| Luffa acutangula | 26 | Angled | Chinese okra | Stokes Seeds Ltd, | Food |
| (L.) Roxb. | | Loofah | | ON, Canada | |
| | | | | Stokes Seeds INC., | |
| | | | | NY, USA, #422Y | |
| Tribe Cucurbiteae | | | | | |
| Genus Cucurbita | | | | | |
| Cucurbita moschata | 40 | Squash & | Butternut | Argentina | Food |
| Duch. ex Poir | | pumpkin | squash* | | |
| Cucurbita pepo L. | 40 | Squash & | Jack | Mckenzie, MB, | Food & |
| C. pepo L. var. pepo L. | | pumpkin | O'Lantern | Canada, #101556 | ornamental |
| | | | Pumpkin* | Canada | |
| C. pepo L. var. turbinate | | | Acorn squash* | Mexico | |
| C.pepo L. var. fastigata | | | Spaghetti | Mexico | |
| | | | squash* | | |

*Fresh product.
¹Taxonomic classification is derived from Schaefer and Renner, 2011.

TABLE 2
Identification of isolated bacterial endophytes with anti-pathogenic activity.

| | | | | 16R rDNA | SEQ |
|---|---|---|---|---|---|
| | Bacterial | | | Genbank | ID |
| Well | isolate | Plant | Source | Accession No. | NO: |
| A′10 | Bacillus | Canary | Fresh | KT281402 | 8 |
| | <EKM213B> | cantaloupe | | | |
| A′3 | Bacillus | Cantaloupe | commercial | KT281397 | 6 |
| | <EKM203B> | | (Delicious) | | |
| A1 | Paenibacillus | Cucumber | commercial | KT281418 | 5 |
| | <EKM101P> | | (Marketmore) | | |
| A4 | Paenibacillus | Cantaloupe | commercial | KT281427 | 7 |
| | <EKM211P> | | (Delicious) | | |
| B′10 | Bacillus | Honeydew | Fresh | KT281404 | 13 |
| | <EKM214B> | | | | |
| B′3 | Bacillus | Cantaloupe | commercial | KT281400 | 11 |
| | <EKM204B> | | (Delicious) | | |
| B1 | Paenibacillus | Cucumber | commercial | KT281417 | 9 |
| | <EKM102P> | | (Marketmore) | | |
| B3 | Paenibacillus | Cantaloupe | commercial | KT281429 | 10 |
| | <EKM204P> | | (Delicious) | | |
| B4 | Paenibacillus | Cantaloupe | commercial | KT281423 | 12 |
| | <EKM212P> | | (Delicious) | | |
| C′3 | Bacillus | Cantaloupe | commercial | KT281395 | 16 |
| | <EKM205B> | | (Delicious) | | |
| C2 | Paenibacillus | Pumpkin | commercial | KT281432 | 14 |
| | <EKM301P> | | (Jack | | |
| | | | Olantern) | | |
| C3 | Paenibacillus | Cantaloupe | commercial | KT281430 | 15 |
| | <EKM205P> | | (Delicious) | | |
| D′3 | Bacillus | Cantaloupe | commercial | KT281399 | 19 |
| | <EKM206B> | | (Delicious) | | |
| D2 | Paenibacillus | Pumpkin | commercial | KT281435 | 17 |
| | <EKM302P> | | (Jack | | |
| | | | Olantern) | | |
| D3 | Paenibacillus | Cantaloupe | commercial | KT281426 | 18 |
| | <EKM206P> | | (Delicious) | | |
| E′3 | Bacillus | Cantaloupe | commercial | KT281398 | 21 |
| | <EKM207B> | | (Delicious) | | |
| E3 | Paenibacillus | Cantaloupe | commercial | KT281421 | 20 |
| | <EKM207P> | | (Delicious) | | |
| F3 | Paenibacillus | Cantaloupe | commercial | KT281431 | 22 |
| | <EKM208P> | | (Delicious) | | |
| G′3 | Bacillus | Cantaloupe | commercial | KT281396 | 24 |
| | <EKM208B> | | (Delicious) | | |
| G2 | Paenibacillus | Cantaloupe | commercial | KT281424 | 23 |
| | <EKM201P> | | (Delicious) | | |
| H2 | Paenibacillus | Cantaloupe | commercial | KT281420 | 25 |
| | <EKM202P> | | (Delicious) | | |
| H3 | Paenibacillus | Cantaloupe | commercial | KT281425 | 26 |
| | <EKM210P> | | (Delicious) | | |
| A′4 | Bacillus | Honeydew | | KT281407 | 27 |
| | <EKM210B> | | | | |
| A′8 | Pesudomonas | Fresh Watermelon | | KT281314 | 29 |
| | <EKM403A> | | | | |
| A5 | Pesudomonas | Field cucumber | | KT281311 | 28 |
| | <EKM103A> | (seed wash) | | | |
| B′1 | Bacillus | Watermelon | | KT281348 | 30 |
| | <EKM410B> | (Crimson sweet) | | | |
| B′4 | Bacillus | Honeydew | | KT281405 | 31 |
| | <EKM211B> | | | | |
| B′5 | Micrococcus | Acorn squash | | KT281322 | 32 |
| | <EKM501U> | | | | |
| B′6 | Bacillus | Spaghetti squash | | KT281363 | 34 |
| | <EKM505B> | | | | |
| B′7 | Microbacterium | Field cucumber | | KT281306 | 35 |
| | <EKM101T> | | | | |
| B′8 | Staphylococcus | Honeydew | | KT281320 | 36 |
| | <EKM201S> | | | | |
| B′9 | Bacillus | Fresh Pumpkin | | KT281338 | 37 |
| | <EKM306B> | | | | |
| B6 | Unclassified | Field cucumber | | KT281287 | 33 |
| | Enterobacteriaceae | (seed wash) | | | |
| | <EKM102Z> | | | | |
| C′1 | Bacillus | Cucumber | | KT281391 | 38 |
| | <EKM126B> | (Marketmore) | | | |
| C′7 | Microbacterium | Field cucumber | | KT281307 | 40 |
| | <EKM102T> | | | | |
| C′8 | Paenibacillus | Fresh Pumpkin | | KT281438 | 41 |
| | <EKM305P> | | | | |
| C′9 | Unclassified | Fresh Pumpkin | | KT281289 | 42 |
| | Enterobacteriaceae | | | | |
| | <EKM301Z> | | | | |
| C5 | Lactococcus | Cucumber (straight | | KT281446 | 39 |
| | <EKM102L> | eight) | | | |
| D′4 | Bacillus | Bottle gourd | | KT222781 | 44 |
| | <EKM702B> | | | | |
| D′5 | Bacillus | Acorn squash | | KT281361 | 45 |
| | <EKM503B> | | | | |
| D′8 | Paenibacillus | Fresh Pumpkin | | KT281437 | 46 |
| | <EKM306P> | | | | |
| D′9 | Unclassified | Fresh Pumpkin | | KT281290 | 47 |
| | Enterobacteriaceae | | | | |
| | <EKM301K> | | | | |
| D10 | Bacillus | Cucumber | | KT281388 | 48 |
| | <EKM119B> | (Marketmore) | | | |
| D4 | Pantoea | Field cucumber | | KT281315 | 43 |
| | <EKM101V> | (seed wash) | | | |
| E′4 | Bacillus | Bottle gourd | | KT220265 | 50 |
| | <EKM703B> | | | | |
| E′5 | Unclassified | Spaghetti squash | | KT281305 | 51 |
| | Paenibacillaceae | | | | |
| | <EKM501M> | | | | |
| E′6 | Unclassified | Chinese okra | | KT222784 | 52 |
| | Enterobacteriaceae | | | | |
| | <EKM602Z> | | | | |
| E′9 | Unclassified | Fresh Pumpkin | | KT281291 | 53 |
| | Enterobacteriaceae | | | | |
| | <EKM302K> | | | | |
| E2 | Paenibacillus | Pumpkin (Jack | | KT281434 | 49 |
| | <EKM303P> | O'lantern) | | | |
| F7 | Pesudomonas | Fresh Watermelon | | KT281313 | 57 |
| | <EKM402A> | | | | |
| F′8 | Paenibacillus | Fresh Pumpkin | | KT281441 | 58 |
| | <EKM308P> | | | | |
| F′9 | Paenibacillus | Fresh Pumpkin | | KT281443 | 59 |
| | <EKM312P> | | | | |
| F4 | Pantoea | Field cucumber | | KT281317 | 54 |
| | <EKM103V> | (seed wash) | | | |
| F5 | Lactococcus | Fresh cantaloupe | | KT281327 | 55 |
| | <EKM203L> | | | | |
| F6 | Unclassified | Cucumber | | KT281298 | 56 |
| | Enterobacteriaceae | (spacemaster) | | | |
| | <EKM102E> | | | | |
| G′5 | Lactococcus | Acorn squash | | KT281328 | 61 |
| | <EKM501L> | | | | |
| G5 | Pediococcus | Fresh cantaloupe | | KT281330 | 60 |
| | <EKM201D> | | | | |
| H′1 | Bacillus | Butternut | | KT281360 | 62 |
| | <EKM502B> | | | | |
| H′2 | Bacillus | Watermelon | | KT281355 | 63 |
| | <EKM417B> | (Crimson sweet) | | | |
| H′5 | Lactococcus | Acorn squash | | KT281329 | 65 |
| | <EKM502L> | | | | |
| H′7 | Bacillus | Fresh Watermelon | | KT281358 | 66 |
| | <EKM419B> | | | | |
| H′8 | Paenibacillus | Fresh Pumpkin | | KT281442 | 67 |
| | <EKM310P> | | | | |
| H5 | Pediococcus | Fresh cantaloupe | | KT281331 | 64 |
| | <EKM202D> | | | | |

TABLE 3
In vitro testing and characterization of isolates having activity against Fusarium graminearum.

| | Growth on | ACC | Phosphate | Auxin | Siderophore | Cellulase | Pectinase | Protease |
|---|---|---|---|---|---|---|---|---|
| Isolate | N-free media | deaminase | solubilization | production | production | activity | activity | activity |
| Bacillus | − | − | − | − | − | − | + | + |
| <EKM203B> | | | | | | | | |
| Bacillus | − | − | − | − | − | N | + | + |
| <EKM204B> | | | | | | | | |
| Bacillus | − | − | − | − | − | N | + | + |
| <EKM205B> | | | | | | | | |
| Bacillus | − | − | − | − | − | N | + | + |
| <EKM206B> | | | | | | | | |
| Bacillus | − | + | − | − | − | N | + | + |
| <EKM207B> | | | | | | | | |
| Bacillus | + | + | − | − | − | N | + | + |
| <EKM208B> | | | | | | | | |
| Bacillus | + | − | − | − | − | N | + | + |
| <EKM213B> | | | | | | | | |
| Bacillus | + | + | − | − | − | N | + | + |
| <EKM214B> | | | | | | | | |
| Paenibacillus | − | − | − | − | − | + | + | + |
| <EKM101P> | | | | | | | | |
| Paenibacillus | − | − | − | − | − | + | + | + |
| <EKM102P> | | | | | | | | |
| Paenibacillus | + | − | − | − | − | + | + | + |
| <EKM201P> | | | | | | | | |
| Paenibacillus | + | − | − | − | − | + | + | + |
| <EKM202P> | | | | | | | | |
| Paenibacillus | − | − | − | − | − | + | + | + |
| <EKM204P> | | | | | | | | |
| Paenibacillus | + | − | − | − | − | + | + | + |
| <EKM205P> | | | | | | | | |
| Paenibacillus | − | − | − | − | − | + | + | + |
| <EKM206P> | | | | | | | | |
| Paenibacillus | − | − | − | − | − | + | + | + |
| <EKM207P> | | | | | | | | |
| Paenibacillus | − | − | − | − | − | + | + | + |
| <EKM208P> | | | | | | | | |
| Paenibacillus | + | − | − | − | − | + | + | + |
| <EKM210P> | | | | | | | | |
| Paenibacillus | − | − | − | − | − | + | + | + |
| <EKM211P> | | | | | | | | |
| Paenibacillus | − | − | − | − | − | + | N | + |
| <EKM212P> | | | | | | | | |
| Paenibacillus | − | − | − | − | − | + | N | + |
| <EKM301P> | | | | | | | | |
| Paenibacillus | − | − | − | − | − | + | + | + |
| <EKM302P> | | | | | | | | |

(+) indicates that the microbe grew and gave a positive result (through the production of certain color, clear zone around the colonies or OD readings),
(−) indicates a negative result in that the microbe grew but did not produce the targeted tested substance (e.g. extracellular enzymes, IAA, etc.);
(N) indicates that the microbe did not grow on the corresponding medium for each test (No growth).

TABLE 4
Identification and characterization of bacterial endophytes with activity against Fusarium graminearum and other
pathogens.

| | | Acetoin & | | | | | | | | | | | | | | | | |
|---|---|---|---|---|---|---|---|---|---|---|---|---|---|---|---|---|---|---|
| | RNase | diacetyl | Fusarium graminearum | | | | Rhizoctonia solani | | | | Phytophthora capsici | | | | Pythium aphanideratum | | | |
| Isolate | activity | production | R1 | R2 | R3 | Mean | R1 | R2 | R3 | Mean | R1 | R2 | R3 | Mean | R1 | R2 | R3 | Mean |
| Bacillus | + | − | 1.7 | 1.6 | 1.6 | 1.63 | 3.2 | 3.1 | 3 | 3.10 | 1.8 | 1.8 | 1.85 | 1.82 | 0 | 0 | 0 | 0.00 |
| <EKM203B> | | | | | | | | | | | | | | | | | | |
| Bacillus | + | + | 1.4 | 1.4 | 1.3 | 1.37 | 3 | 2.8 | 2.8 | 2.87 | 1.6 | 1.6 | 1.65 | 1.62 | 0 | 0 | 0 | 0.00 |
| <EKM204B> | | | | | | | | | | | | | | | | | | |
| Bacillus | + | + | 1.6 | 1.5 | 1.4 | 1.50 | 3 | 3 | 3.2 | 3.07 | 1.8 | 1.8 | 1.8 | 1.80 | 0 | 0 | 0 | 0.00 |
| <EKM205B> | | | | | | | | | | | | | | | | | | |
| Bacillus | + | + | 1.6 | 1.5 | 1.4 | 1.50 | 2.9 | 2.9 | 2.9 | 2.90 | 1.7 | 1.75 | 1.7 | 1.72 | 0 | 0 | 0 | 0.00 |
| <EKM206B> | | | | | | | | | | | | | | | | | | |
| Bacillus | + | + | 1.6 | 1.55 | 1.5 | 1.55 | 2.7 | 2.9 | 3 | 2.87 | 1.65 | 1.7 | 1.7 | 1.68 | 0 | 0 | 0 | 0.00 |
| <EKM207B> | | | | | | | | | | | | | | | | | | |
| Bacillus | + | + | 1.6 | 1.7 | 1.6 | 1.63 | 2.7 | 2.9 | 3.5 | 3.03 | 0 | 0 | 0 | 0.00 | 1.7 | 1.75 | 1.7 | 1.72 |
| <EKM208B> | | | | | | | | | | | | | | | | | | |
| Bacillus | + | + | 2 | 2 | 2.1 | 2.03 | 3.7 | 3.7 | 3.8 | 3.73 | 1.4 | 1.5 | 1.4 | 1.43 | 0 | 0 | 0 | 0.00 |
| <EKM213B> | | | | | | | | | | | | | | | | | | |
| Bacillus | + | + | 2.05 | 2 | 2.1 | 2.05 | 3.8 | 3.8 | 3.5 | 3.70 | 1.5 | 1.5 | 1.35 | 1.45 | 0 | 0 | 0 | 0.00 |
| <EKM214B> | | | | | | | | | | | | | | | | | | |
| Paenibacillus | + | + | 1.85 | 1.8 | 1.85 | 1.83 | 3.2 | 3.1 | 3 | 3.10 | 1.85 | 1.9 | 1.8 | 1.85 | 0 | 0 | 0 | 0.00 |
| <EKM101P> | | | | | | | | | | | | | | | | | | |
| Paenibacillus | + | + | 1.9 | 1.85 | 1.9 | 1.88 | 3.55 | 3.3 | 3 | 3.28 | 2 | 2 | 2 | 2.00 | 0 | 0 | 0 | 0.00 |
| <EKM102P> | | | | | | | | | | | | | | | | | | |
| Paenibacillus | − | + | 1.4 | 1.4 | 1.5 | 1.43 | 3.2 | 3 | 3.3 | 3.17 | 2.1 | 2.1 | 2.1 | 2.10 | 1.3 | 1.4 | 1.3 | 1.33 |
| <EKM201P> | | | | | | | | | | | | | | | | | | |
| Paenibacillus | + | + | 1.5 | 1.55 | 1.55 | 1.53 | 3.2 | 3.4 | 3.3 | 3.30 | 2.2 | 2.2 | 2.2 | 2.20 | 1.3 | 1.4 | 1.3 | 1.33 |
| <EKM202P> | | | | | | | | | | | | | | | | | | |
| Paenibacillus | + | + | 1.7 | 1.7 | 1.7 | 1.70 | 2.9 | 3 | 3 | 2.97 | 2.1 | 2.1 | 2.1 | 2.10 | 0 | 0 | 0 | 0.00 |
| <EKM204P> | | | | | | | | | | | | | | | | | | |
| Paenibacillus | + | + | 1.9 | 1.9 | 1.9 | 1.90 | 3.2 | 3.2 | 3.2 | 3.20 | 1.95 | 1.95 | 2 | 1.97 | 0 | 0 | 0 | 0.00 |
| <EKM205P> | | | | | | | | | | | | | | | | | | |
| Paenibacillus | + | + | 2 | 2 | 2 | 2.00 | 3.5 | 3.6 | 3.5 | 3.53 | 1.8 | 1.85 | 1.85 | 1.83 | 0 | 0 | 0 | 0.00 |
| <EKM206P> | | | | | | | | | | | | | | | | | | |
| Paenibacillus | + | + | 1.6 | 1.6 | 1.6 | 1.60 | 3.1 | 2.9 | 3.2 | 3.07 | 2 | 2 | 2 | 2.00 | 0 | 0 | 0 | 0.00 |
| <EKM207P> | | | | | | | | | | | | | | | | | | |
| Paenibacillus | + | + | 1.9 | 1.85 | 1.9 | 1.88 | 3 | 3.1 | 3.4 | 3.17 | 1.8 | 1.8 | 1.75 | 1.78 | 0 | 0 | 0 | 0.00 |
| <EKM208P> | | | | | | | | | | | | | | | | | | |
| Paenibacillus | − | + | 1.6 | 1.6 | 1.6 | 1.60 | 2.6 | 2.5 | 3 | 2.70 | 2 | 1.95 | 2 | 1.98 | 0 | 0 | 0 | 0.00 |
| <EKM210P> | | | | | | | | | | | | | | | | | | |
| Paenibacillus | + | + | 1.8 | 1.75 | 1.8 | 1.78 | 3 | 3.2 | 3.1 | 3.10 | 2.2 | 2.2 | 2.2 | 2.20 | 0 | 0 | 0 | 0.00 |
| <EKM211P> | | | | | | | | | | | | | | | | | | |
| Paenibacillus | − | + | 1.75 | 1.7 | 1.7 | 1.72 | 3.4 | 3.5 | 3.5 | 3.47 | 2.1 | 2.1 | 2.1 | 2.10 | 1.4 | 1.4 | 1.4 | 1.40 |
| <EKM212P> | | | | | | | | | | | | | | | | | | |
| Paenibacillus | − | + | 1.9 | 1.9 | 1.9 | 1.90 | 3.2 | 3.9 | 3.2 | 3.43 | 2.4 | 2.35 | 2.35 | 2.37 | 0 | 0 | 0 | 0.00 |
| <EKM301P> | | | | | | | | | | | | | | | | | | |
| Paenibacillus | − | + | 1.8 | 1.8 | 1.8 | 1.80 | 3.2 | 3.5 | 3.2 | 3.30 | 2.4 | 2.4 | 2.4 | 2.40 | 0 | 0 | 0 | 0.00 |
| <EKM302P> | | | | | | | | | | | | | | | | | | |

TABLE 5
In vitro testing and characterization of isolates having activity against powdery mildew (+) indicates a positive
result, (−) indicates a negative result, (N) indicates no growth.

| | Growth on | ACC | Phosphate | Auxin | Siderophore | Cellulase | Pectinase | Protease |
|---|---|---|---|---|---|---|---|---|
| Isolate | N-free media | deaminase | solubilization | production | production | activity | activity | activity |
| Bacillus <EKM119B> | − | − | − | − | − | − | N | − |
| Bacillus <EKM126B> | − | + | − | − | − | N | N | + |
| Bacillus <EKM210B> | − | − | + | + | − | − | − | − |
| Bacillus <EKM211B> | − | − | − | − | − | − | − | + |
| Bacillus <EKM306B> | + | − | − | + | − | N | N | + |
| Bacillus <EKM410B> | − | − | + | + | + | − | − | + |
| Bacillus <EKM417B> | − | − | − | − | − | + | + | + |
| Bacillus <EKM419B> | − | − | − | + | − | N | N | + |
| Bacillus <EKM502B> | + | + | − | + | − | N | N | N |
| Bacillus <EKM503B> | − | − | − | + | N | N | N | + |
| Bacillus <EKM505B> | − | − | + | + | − | − | − | − |
| Bacillus <EKM702B> | − | − | − | − | N | N | N | N |
| Bacillus <EKM703B> | − | − | − | − | N | N | N | N |
| Lactococcus | − | − | + | + | − | N | − | − |
| <EKM102L> | | | | | | | | |
| Lactococcus | − | − | + | N | N | N | N | − |
| <EKM203L> | | | | | | | | |
| Lactococcus | − | − | + | + | − | − | − | − |
| <EKM501L> | | | | | | | | |
| Lactococcus | − | − | + | − | N | N | N | − |
| <EKM502L> | | | | | | | | |
| Microbacterium | − | − | − | + | − | N | N | − |
| <EKM101T> | | | | | | | | |
| Microbacterium | − | − | − | − | − | N | N | − |
| <EKM102T> | | | | | | | | |
| Micrococcus | − | − | + | + | + | − | − | − |
| <EKM501U> | | | | | | | | |
| Paenibacillus | − | − | − | N | N | N | N | N |
| <EKM303P> | | | | | | | | |
| Paenibacillus | − | − | N | N | N | N | N | N |
| <EKM305P> | | | | | | | | |
| Paenibacillus | − | − | N | N | N | N | N | N |
| <EKM306P> | | | | | | | | |
| Paenibacillus | − | − | N | N | N | N | N | N |
| <EKM308P> | | | | | | | | |
| Paenibacillus | − | − | N | N | N | N | N | N |
| <EKM310P> | | | | | | | | |
| Paenibacillus | − | − | N | N | N | N | N | N |
| <EKM312P> | | | | | | | | |
| Pantoea <EKM101V> | − | − | − | + | + | − | − | − |
| Pantoea <EKM103V> | − | N | − | + | + | − | − | − |
| Pediococcus | − | − | + | N | − | − | − | − |
| <EKM201D> | | | | | | | | |
| Pediococcus | − | − | + | − | − | − | − | − |
| <EKM202D> | | | | | | | | |
| Pesudomonas | − | − | − | − | − | − | N | N |
| <EKM103A> | | | | | | | | |
| Pesudomonas | − | − | + | + | + | − | − | − |
| <EKM402A> | | | | | | | | |
| Pesudomonas | − | − | N | − | N | N | N | N |
| <EKM403A> | | | | | | | | |
| Staphylococcus | − | − | − | − | − | − | − | N |
| <EKM201S> | | | | | | | | |
| Unclassified | − | N | + | + | − | − | − | − |
| Enterobacteriaceae | | | | | | | | |
| <EKM102E> | | | | | | | | |
| Unclassified | − | − | − | + | − | − | − | − |
| Enterobacteriaceae | | | | | | | | |
| <EKM102Z> | | | | | | | | |
| Unclassified | − | − | + | + | − | − | − | − |
| Enterobacteriaceae | | | | | | | | |
| <EKM301K> | | | | | | | | |
| Unclassified | − | − | + | + | + | − | − | − |
| Enterobacteriaceae | | | | | | | | |
| <EKM301Z> | | | | | | | | |
| Unclassified | − | − | + | + | − | − | − | − |
| Enterobacteriaceae | | | | | | | | |
| <EKM302K> | | | | | | | | |
| Unclassified | − | − | + | + | + | − | N | + |
| Enterobacteriaceae | | | | | | | | |
| <EKM602Z> | | | | | | | | |
| Unclassified | − | − | − | + | N | N | N | N |
| Paenibacillaceae | | | | | | | | |
| <EKM501M> | | | | | | | | |

TABLE 6
Identification and characterization of bacterial endophytes that have activity against plant pathogens.

| | | Acetoin & | Fusarium | | | | Rhizoctonia | | | | Phytophthora | | | | Pythium | | | |
|---|---|---|---|---|---|---|---|---|---|---|---|---|---|---|---|---|---|---|
| | RNase | diacetyl | graminearum | | | | solani | | | | capsici | | | | aphanideratum | | | |
| Isolate | activity | production | R1 | R2 | R3 | Mean | R1 | R2 | R3 | Mean | R1 | R2 | R3 | Mean | R1 | R2 | R3 | Mean |
| Bacillus | + | + | 0 | 0 | 0 | 0 | 0 | 0 | 0 | 0 | 0 | 0 | 0 | 0 | 0 | 0 | 0 | 0 |
| <EKM119B> | | | | | | | | | | | | | | | | | | |
| Bacillus | + | + | 0 | 0 | 0 | 0 | 0 | 0 | 0 | 0 | 0 | 0 | 0 | 0 | 0 | 0 | 0 | 0 |
| <EKM126B> | | | | | | | | | | | | | | | | | | |
| Bacillus | + | − | 0 | 0 | 0 | 0 | 0 | 0 | 0 | 0 | 0 | 0 | 0 | 0 | 0 | 0 | 0 | 0 |
| <EKM210B> | | | | | | | | | | | | | | | | | | |
| Bacillus | + | + | 0 | 0 | 0 | 0 | 0 | 0 | 0 | 0 | 0 | 0 | 0 | 0 | 0 | 0 | 0 | 0 |
| <EKM211B> | | | | | | | | | | | | | | | | | | |
| Bacillus | + | − | 0 | 0 | 0 | 0 | 0 | 0 | 0 | 0 | 0 | 0 | 0 | 0 | 0 | 0 | 0 | 0 |
| <EKM306B> | | | | | | | | | | | | | | | | | | |
| Bacillus | + | + | 0 | 0 | 0 | 0 | 0 | 0 | 0 | 0 | 0 | 0 | 0 | 0 | 0 | 0 | 0 | 0 |
| <EKM410B> | | | | | | | | | | | | | | | | | | |
| Bacillus | + | + | 0 | 0 | 0 | 0 | 0 | 0 | 0 | 0 | 3 | 3 | 3 | 3 | 0 | 0 | 0 | 0 |
| <EKM417B> | | | | | | | | | | | | | | | | | | |
| Bacillus | + | − | 0 | 0 | 0 | 0 | 0 | 0 | 0 | 0 | 0 | 0 | 0 | 0 | 0 | 0 | 0 | 0 |
| <EKM419B> | | | | | | | | | | | | | | | | | | |
| Bacillus | N | + | 0 | 0 | 0 | 0 | 0 | 0 | 0 | 0 | 0 | 0 | 0 | 0 | 0 | 0 | 0 | 0 |
| <EKM502B> | | | | | | | | | | | | | | | | | | |
| Bacillus | + | N | 0 | 0 | 0 | 0 | 0 | 0 | 0 | 0 | 0 | 0 | 0 | 0 | 0 | 0 | 0 | 0 |
| <EKM503B> | | | | | | | | | | | | | | | | | | |
| VBacillus | + | − | 0 | 0 | 0 | 0 | 3.30 | 3.00 | 3.20 | 3.17 | 0 | 0 | 0 | 0 | 0 | 0 | 0 | 0 |
| <EKM505B> | | | | | | | | | | | | | | | | | | |
| Bacillus | N | N | 0 | 0 | 0 | 0 | 2.20 | 2.50 | 2.40 | 2.37 | 0 | 0 | 0 | 0 | 0 | 0 | 0 | 0 |
| <EKM702B> | | | | | | | | | | | | | | | | | | |
| Bacillus | N | N | 0 | 0 | 0 | 0 | 0 | 0 | 0 | 0 | 0 | 0 | 0 | 0 | 0 | 0 | 0 | 0 |
| <EKM703B> | | | | | | | | | | | | | | | | | | |
| Lactococcus | + | − | 0 | 0 | 0 | 0 | 0 | 0 | 0 | 0 | 1.6 | 1.7 | 1.7 | 1.667 | 1.75 | 1.7 | 1.75 | 1.733 |
| <EKM102L> | | | | | | | | | | | | | | | | | | |
| Lactococcus | − | − | 0 | 0 | 0 | 0 | 0 | 0 | 0 | 0 | 1.95 | 1.85 | 1.85 | 1.883 | 1.75 | 1.75 | 1.75 | 1.75 |
| <EKM203L> | | | | | | | | | | | | | | | | | | |
| Lactococcus | + | − | 0 | 0 | 0 | 0 | 0 | 0 | 0 | 0 | 1.7 | 1.8 | 1.8 | 1.767 | 1.8 | 1.8 | 1.75 | 1.783 |
| <EKM501L> | | | | | | | | | | | | | | | | | | |
| Lactococcus | − | − | 0 | 0 | 0 | 0 | 0 | 0 | 0 | 0 | 1.8 | 1.85 | 1.85 | 1.833 | 1.8 | 1.8 | 1.8 | 1.8 |
| <EKM502L> | | | | | | | | | | | | | | | | | | |
| Microbacterium | + | + | 0 | 0 | 0 | 0 | 0 | 0 | 0 | 0 | 0 | 0 | 0 | 0 | 0 | 0 | 0 | 0 |
| <EKM101T> | | | | | | | | | | | | | | | | | | |
| Microbacterium | + | + | 0 | 0 | 0 | 0 | 0 | 0 | 0 | 0 | 0 | 0 | 0 | 0 | 0 | 0 | 0 | 0 |
| <EKM102T> | | | | | | | | | | | | | | | | | | |
| Micrococcus | + | − | 0 | 0 | 0 | 0 | 0 | 0 | 0 | 0 | 0 | 0 | 0 | 0 | 0 | 0 | 0 | 0 |
| <EKM501U> | | | | | | | | | | | | | | | | | | |
| Paenibacillus | − | − | 0 | 0 | 0 | 0 | 0 | 0 | 0 | 0 | 0 | 0 | 0 | 0 | 0 | 0 | 0 | 0 |
| <EKM303P> | | | | | | | | | | | | | | | | | | |
| Paenibacillus | N | N | 0 | 0 | 0 | 0 | 0 | 0 | 0 | 0 | 0 | 0 | 0 | 0 | 0 | 0 | 0 | 0 |
| <EKM305P> | | | | | | | | | | | | | | | | | | |
| Paenibacillus | N | N | 0 | 0 | 0 | 0 | 0 | 0 | 0 | 0 | 0 | 0 | 0 | 0 | 0 | 0 | 0 | 0 |
| <EKM306P> | | | | | | | | | | | | | | | | | | |
| Paenibacillus | N | N | 0 | 0 | 0 | 0 | 0 | 0 | 0 | 0 | 0 | 0 | 0 | 0 | 0 | 0 | 0 | 0 |
| <EKM308P> | | | | | | | | | | | | | | | | | | |
| Paenibacillus | N | N | 0 | 0 | 0 | 0 | 0 | 0 | 0 | 0 | 0 | 0 | 0 | 0 | 0 | 0 | 0 | 0 |
| <EKM310P> | | | | | | | | | | | | | | | | | | |
| Paenibacillus | N | N | 0 | 0 | 0 | 0 | 0 | 0 | 0 | 0 | 0 | 0 | 0 | 0 | 0 | 0 | 0 | 0 |
| <EKM312P> | | | | | | | | | | | | | | | | | | |
| Pantoea | − | + | 0 | 0 | 0 | 0 | 0 | 0 | 0 | 0 | 2.3 | 2.35 | 2.4 | 2.35 | 1.5 | 1.5 | 1.6 | 1.533 |
| <EKM101V> | | | | | | | | | | | | | | | | | | |
| Pantoea | − | + | 0 | 0 | 0 | 0 | 0 | 0 | 0 | 0 | 2.5 | 2.5 | 2.5 | 2.5 | 0 | 0 | 0 | 0 |
| <EKM103V> | | | | | | | | | | | | | | | | | | |
| Pediococcus | − | − | 0 | 0 | 0 | 0 | 2.95 | 3.1 | 2.8 | 2.95 | 1.75 | 1.8 | 1.8 | 1.783 | 0 | 0 | 0 | 0 |
| <EKM201D> | | | | | | | | | | | | | | | | | | |
| Pediococcus | − | − | 0 | 0 | 0 | 0 | 3 | 3.2 | 2.9 | 3.033 | 1.6 | 1.75 | 1.7 | 1.683 | 0 | 0 | 0 | 0 |
| <EKM202D> | | | | | | | | | | | | | | | | | | |
| Pesudomonas | + | − | 0 | 0 | 0 | 0 | 0 | 0 | 0 | 0 | 0 | 0 | 0 | 0 | 0 | 0 | 0 | 0 |
| <EKM103A> | | | | | | | | | | | | | | | | | | |
| Pesudomonas | + | + | 0 | 0 | 0 | 0 | 0 | 0 | 0 | 0 | 0 | 0 | 0 | 0 | 0 | 0 | 0 | 0 |
| <EKM402A> | | | | | | | | | | | | | | | | | | |
| Pesudomonas | N | + | 0 | 0 | 0 | 0 | 0 | 0 | 0 | 0 | 0 | 0 | 0 | 0 | 0 | 0 | 0 | 0 |
| <EKM403A> | | | | | | | | | | | | | | | | | | |
| Staphylococcus | − | + | 0 | 0 | 0 | 0 | 0 | 0 | 0 | 0 | 0 | 0 | 0 | 0 | 0 | 0 | 0 | 0 |
| <EKM201S> | | | | | | | | | | | | | | | | | | |
| Unclassified | − | + | 0 | 0 | 0 | 0 | 0 | 0 | 0 | 0 | 2.7 | 2.7 | 2.7 | 2.7 | 1.8 | 1.6 | 1.8 | 1.733 |
| Enterobacteriaceae | | | | | | | | | | | | | | | | | | |
| <EKM102E> | | | | | | | | | | | | | | | | | | |
| Unclassified | − | + | 0 | 0 | 0 | 0 | 0 | 0 | 0 | 0 | 0 | 0 | 0 | 0 | 0 | 0 | 0 | 0 |
| Enterobacteriaceae | | | | | | | | | | | | | | | | | | |
| <EKM102Z> | | | | | | | | | | | | | | | | | | |
| Unclassified | + | − | 0 | 0 | 0 | 0 | 0 | 0 | 0 | 0 | 0 | 0 | 0 | 0 | 0 | 0 | 0 | 0 |
| Enterobacteriaceae | | | | | | | | | | | | | | | | | | |
| <EKM301K> | | | | | | | | | | | | | | | | | | |
| Unclassified | + | − | 0 | 0 | 0 | 0 | 0 | 0 | 0 | 0 | 0 | 0 | 0 | 0 | 0 | 0 | 0 | 0 |
| Enterobacteriaceae | | | | | | | | | | | | | | | | | | |
| <EKM301Z> | | | | | | | | | | | | | | | | | | |
| Unclassified | + | − | 0 | 0 | 0 | 0 | 0 | 0 | 0 | 0 | 0 | 0 | 0 | 0 | 0 | 0 | 0 | 0 |
| Enterobacteriaceae | | | | | | | | | | | | | | | | | | |
| <EKM302K> | | | | | | | | | | | | | | | | | | |
| Unclassified | + | + | 0 | 0 | 0 | 0 | 0 | 0 | 0 | 0 | 0 | 0 | 0 | 0 | 0 | 0 | 0 | 0 |
| Enterobacteriaceae | | | | | | | | | | | | | | | | | | |
| <EKM602Z> | | | | | | | | | | | | | | | | | | |
| Unclassified | N | N | 0 | 0 | 0 | 0 | 0 | 0 | 0 | 0 | 0 | 0 | 0 | 0 | 0 | 0 | 0 | 0 |
| Paenibacillaceae | | | | | | | | | | | | | | | | | | |
| <EKM501M> | | | | | | | | | | | | | | | | | | |

TABLE 7
Identification and characterization of bacterial
endophytes with activity against powdery mildew.

| | 16S rDNA | DI % | PE % | DI % | PE % |
|---|---|---|---|---|---|
| | Genbank | (Leaf | (Leaf | (Whole | (Whole |
| Isolate name | Accession No. | disc) | disc) | leaf) | leaf) |
| Bacillus | KT281388 | 28.57 | 64.87 | 42.86 | −15.39 |
| <EKM119B> | | | | | |
| Bacillus | KT281391 | 26.66 | 67.21 | 31.43 | 15.38 |
| <EKM126B> | | | | | |
| Bacillus | KT281407 | 28.57 | 64.87 | 31.43 | 15.38 |
| <EKM210B> | | | | | |
| Bacillus | KT281405 | 28.57 | 64.87 | 37.14 | −0.01 |
| <EKM211B> | | | | | |
| Bacillus | KT281338 | 16.19 | 80.09 | 28.57 | 23.07 |
| <EKM306B> | | | | | |
| Bacillus | KT281348 | 27.61 | 66.05 | 40.00 | −7.70 |
| <EKM410B> | | | | | |
| Bacillus | KT281355 | 21.90 | 73.25 | 31.43 | 15.38 |
| <EKM417B> | | | | | |
| Bacillus | KT281358 | 8.57 | 89.46 | 22.86 | 38.46 |
| <EKM419B> | | | | | |
| Bacillus | KT281360 | 26.66 | 54.67 | 45.71 | −23.09 |
| <EKM502B> | | | | | |
| Bacillus | KT281361 | 27.61 | 64.34 | 37.14 | −0.01 |
| <EKM503B> | | | | | |
| Bacillus | KT281363 | 23.80 | 70.73 | 25.71 | 30.76 |
| <EKM505B> | | | | | |
| Bacillus | KT222781 | 24.76 | 69.55 | 42.86 | −15.39 |
| <EKM702B> | | | | | |
| Bacillus | KT220265 | 20.95 | 74.24 | 31.43 | 15.38 |
| <EKM703B> | | | | | |
| Lactococcus | KT281446 | 24.76 | 69.55 | 68.57 | −84.63 |
| <EKM102L> | | | | | |
| Lactococcus | KT281327 | 20.95 | 74.24 | 34.29 | 7.69 |
| <EKM203L> | | | | | |
| Lactococcus | KT281328 | 10.47 | 87.12 | 34.29 | 7.69 |
| <EKM501L> | | | | | |
| Lactococcus | KT281329 | 18.09 | 77.75 | 31.43 | 15.38 |
| <EKM502L> | | | | | |
| Microbacterium | KT281306 | 26.66 | 67.21 | 34.29 | 7.69 |
| <EKM101T> | | | | | |
| Microbacterium | KT281307 | 21.90 | 73.07 | 37.14 | −0.01 |
| <EKM102T> | | | | | |
| Micrococcus | KT281322 | 26.66 | 67.21 | 28.57 | 23.07 |
| <EKM501U> | | | | | |
| Paenibacillus | KT281434 | 26.66 | 67.21 | 42.86 | −15.39 |
| <EKM303P> | | | | | |
| Paenibacillus | KT281438 | 27.61 | 66.05 | 28.57 | 23.07 |
| <EKM305P> | | | | | |
| Paenibacillus | KT281437 | 20.95 | 74.24 | 37.14 | −0.01 |
| <EKM306P> | | | | | |
| Paenibacillus | KT281441 | 15.23 | 81.27 | 31.43 | 15.38 |
| <EKM308P> | | | | | |
| Paenibacillus | KT281442 | 29.52 | 63.70 | 31.43 | 15.38 |
| <EKM310P> | | | | | |
| Paenibacillus | KT281443 | 10.47 | 87.12 | 31.43 | 15.38 |
| <EKM312P> | | | | | |
| Pantoea | KT281315 | 9.52 | 88.29 | 25.71 | 30.76 |
| <EKM101V> | | | | | |
| Pantoea | KT281317 | 21.90 | 73.07 | 22.86 | 38.46 |
| <EKM103V> | | | | | |
| Pediococcus | KT281330 | 14.28 | 82.44 | 20.00 | 46.15 |
| <EKM201D> | | | | | |
| Pediococcus | KT281331 | 8.57 | 89.46 | 28.57 | 23.07 |
| <EKM202D> | | | | | |
| Pesudomonas | KT281311 | 28.57 | 64.87 | 28.57 | 23.07 |
| <EKM103A> | | | | | |
| Pesudomonas | KT281313 | 31.42 | 61.36 | 42.86 | −15.39 |
| <EKM402A> | | | | | |
| Pesudomonas | KT281314 | 10.47 | 87.12 | 45.71 | −23.09 |
| <EKM403A> | | | | | |
| Staphylococcus | KT281320 | 19.04 | 76.58 | 25.71 | 30.76 |
| <EKM201S> | | | | | |
| Unclassified | KT281298 | 13.33 | 83.60 | 42.86 | −15.39 |
| Enterobacteriaceae | | | | | |
| <EKM102E> | | | | | |
| Unclassified | KT281287 | 24.76 | 69.55 | 22.86 | 38.46 |
| Enterobacteriaceae | | | | | |
| <EKM102Z> | | | | | |
| Unclassified | KT281290 | 11.42 | 85.95 | 25.71 | 30.76 |
| Enterobacteriaceae | | | | | |
| <EKM301K> | | | | | |
| Unclassified | KT281289 | 12.38 | 84.77 | 31.43 | 15.38 |
| Enterobacteriaceae | | | | | |
| <EKM301Z> | | | | | |
| Unclassified | KT281291 | 20.00 | 75.40 | 28.57 | 23.07 |
| Enterobacteriaceae | | | | | |
| <EKM302K> | | | | | |
| Unclassified | KT222784 | 17.14 | 78.92 | 40.00 | −7.70 |
| Enterobacteriaceae | | | | | |
| <EKM602Z> | | | | | |
| Unclassified | KT281305 | 19.04 | 76.58 | 28.57 | 23.07 |
| Paenibacillaceae | | | | | |
| <EKM501M> | | | | | |

### REFERENCES CITED

1. Bosch T C G, McFall-Ngai M J: Metaorganisms as the new frontier. Zoology 2011, 114:181. Bosch T C G, McFall-Ngai M J: Metaorganisms as the new frontier. Zoology 2011, 114:185-190.

2. Turner T R, James E K, Poole P S: The plant microbiome. Genome Biol 2013, 14.

3. Knief C: Analysis of plant microbe interactions in the era of next generation sequencing technologies. Front Plant Sci 2014, 5.

4. Truyens S, Weyens N, Cuypers A, Vangronsveld J: Bacterial seed endophytes: genera, vertical transmission and interaction with plants: Bacterial seed endophytes. Environ Microbiol Rep 2015, 7:40-50.

5. Glassner H, Zchori-Fein E, Compant S, Sessitsch A, Katzir N, Portnoy V, Yaron S: Characterization of endophytic bacteria from cucurbit fruits with potential benefits to agriculture in melons (Cucumis melo L.). FEMS Microbiol Ecol 2015, 91:fiv074.

6. Ryan R P, Germaine K, Franks A, Ryan D J, Dowling D N: Bacterial endophytes: recent developments and applications. FEMS Microbiol Lett 2008, 278:1-9.

7. Reinhold-Hurek B, Hurek T: Living inside plants: bacterial endophytes. Curr Opin Plant Biol 2011, 14:435-443.

8. Zhang H W, Song Y C, Tan R X: Biology and chemistry of endophytes. Nat Prod Rep 2006, 23:753.

9. Hardoim P R, van Overbeek L S, Elsas J D van: Properties of bacterial endophytes and their proposed role in plant growth. Trends Microbiol 2008, 16:463-471.

10. Bright M, Bulgheresi S: A complex journey: transmission of microbial symbionts. Nat Rev Microbiol 2010, 8:218-230.

11. Zilber-Rosenberg I, Rosenberg E: Role of microorganisms in the evolution of animals and plants: the hologenome theory of evolution. FEMS Microbiol Rev 2008, 32:723-735.

12. Berg G: Plant-microbe interactions promoting plant growth and health: perspectives for controlled use of microorganisms in agriculture. Appl Microbiol Biotechnol 2009, 84:11-18.

13. Bulgarelli D, Schlaeppi K, Spaepen S, van Themaat E V L, Schulze-Lefert P: Structure and functions of the bacterial microbiota of plants. Annu Rev Plant Biol 2013, 64:807-838.

14. Glick B R, Cheng Z, Czarny J, Duan J: Promotion of plant growth by ACC deaminase-producing soil bacteria. Eur J Plant Pathol 2007,119:329-339.

15. Lugtenberg B J J, Chin-A-Woeng T F C, Bloemberg G V: Microbe-plant interactions: principles and mechanisms. Antonie Van Leeuwenhoek 2002, 81:373-383.

16. Flemming H -C, Wingender J: The biofilm matrix. Nat Rev Microbiol 2010.

17. Chee-Sanford J C, Williams II M M, Davis A S, Sims G K: Do microorganisms influence seed-bank dynamics? Weed Sci 2006, 54:575-587.

18. Schaefer H, Renner S S: Cucurbitaceae. In The Families and Genera of Vascular Plants. Flowering Plants. Eudicots: Sapindales, Cucurbitales, Myrtaceae. Volume X. 1st edition. Edited by Kubitzki K. Springer-Verlag Berlin Heidelberg; 2010:112-174.

19. Schaefe H, Renner S S: Phylogenetic relationships in the order Cucurbitales and a new classification of the gourd family (Cucurbitaceae). TAXON February 20 11,60:122-138.

20. Heneidak S, Khalik K A: Seed coat diversity in some tribes of Cucurbitaceae: implications for taxonomy and species identification. Acta Bot Bras 2015, 29:129-142.

21. Brothwell D, Brothwell P: Food in Antiquity: A Survey of the Diet of Early Peoples. New York: Fredrick A. Praeger; 1969.

22. Robinson R W, Decker-Walters D S: Cucurbits (Crop Production Science in Horticulture). 1st ed. Wallingford, Oxon, U.K.: CAB international; 1997.

23. Lebeda A, Widrlechner M P, Staub J, Ezura H, Zalapa J, Kristkova E: Cucurbits (Cucurbitaceae; Cucumis spp., Cucurbita Spp., Citrullus Spp.). In Genetic resources, chromosome engineering, and crop improvement. Volume 3. Edited by Singh RJ. Boca Raton: CRC Press; 2005:272-344.

24. Schaefer H, Heibl C, Renner S S: Gourds afloat: a dated phylogeny reveals an Asian origin of the gourd family (Cucurbitaceae) and numerous oversea dispersal events. Proc R Soc B Biol Sci 2009, 276:843-851.

25. Fümkranz M, Lukesch B, Muller H, Huss H, Grube M, Berg G: Microbial diversity inside pumpkins: Microhabitat-specific communities display a high antagonistic potential against phytopathogens. Microb Ecol 2012,63:418-428.

26. Fëmkranz M, Adam E, Müller H, Grube M, Huss H, Winkler J, Berg G: Promotion of growth, health and stress tolerance of Styrian oil pumpkins by bacterial endophytes. Eur J Plant Pathol 2012, 134:509-519.

27. Hardoim P R, Hardoim C C P, van Overbeek L S, van Elsas J D: Dynamics of Seed-Borne Rice Endophytes on Early Plant Growth Stages. PLoS ONE 2012, 7:e30438.

28. Links M G, Demeke T, Gräfenhan T, Hill J E, Hemmingsen S M, Dumonceaux T J: Simultaneous profiling of seed-associated bacteria and fungi reveals antagonistic interactions between microorganisms within a shared epiphytic microbiome on Triticum and Brassica seeds. New Phytol 2014, 202:542-553.

29. Johnston-Monje D, Raizada M N: Conservation and diversity of seed associated endophytes in Zea across boundaries of evolution, ethnography and ecology. PLoS ONE 2011, 6:e20396.

30. Letunic I, Bork P: Interactive Tree Of Life (iTOL): an online tool for phylogenetic tree display and annotation. Bioinformatics 2007, 23:127-128.

31. Letunic I, Bork P: Interactive Tree Of Life v2: online annotation and display of phylogenetic trees made easy. Nucleic Acids Res 2011, 39(suppl):W475-W478.

32. Chelius M K, Triplett E W: The diversity of Archaea and bacteria in association with the roots of Zea mays L. Microb Ecol 2001, 41:252-263.

33. Cole J R, Wang Q, Fish J A, Chai B, McGarrell D M, Sun Y, Brown C T, Porras-Alfaro A, Kuske C R, Tiedje J M: Ribosomal Database Project: data and tools for high throughput rRNA analysis. Nucleic Acids Res 2014, 42:D633-D642.

34. Hall T: BioEdit: An important software for molecular biology. GERF Bull Biosci 2011, 2:60-61.

35. Tamura K, Stecher G, Peterson D, Filipski A, Kumar S: MEGA6: Molecular Evolutionary Genetics Analysis Version 6.0. Mol Biol Evol 2013, 30:2725-2729.

36. Hino S, Wilson P W: Nitrogen fixation by a facultative Bacillus. J Bacteriol 1958, 75:403.

37. Nautiyal C: An effecient microbiological growth medium for screening phosphate solubilizing microorganisms. FEMS Microbiol Lett 1999, 170:265-270.

38. Bric J M, Bostock R M, Silverstone S E: Rapid in situ assay for indoleacetic acid production by bacteria immobilized on a nitrocellulose membrane. Appl Environ Microbiol 1991, 57:535-538.

39. Cox C D: Deferration of laboratory media and assays for ferric and ferrous ions. Methods Enzymol , 235:315-329.

40. Perez-Miranda S, Cabirol N, George-Tellez R, Zamudio-Rivera L S, Fernandez FJ: O-CAS, a fast and universal method for siderophore detection. J Microbiol Methods 2007, 70:127-131.

41. Kasana R C, Salwan R, Dhar H, Dutt S, Gulati A: A rapid and easy method for the detection of microbial cellulases on agar plates using Gram's iodine. Curr Microbiol 2008, 57:503-507.

42. Soares M M, Silva R da, Gomes E: Screening of bacterial strains for pectinolytic activity: characterization of the polygalacturonase produced by Bacillus sp. Rev Microbiol 1999, 30:299-303.

43. Brown M R W, Foster J H S: A simple diagnostic milk medium for Pseudomonas aeruginosa. J Clin Pathol 1970, 23:172-177.

44. Egamberdieva D, Kucharova Z: Selection for root colonising bacteria stimulating wheat growth in saline soils. Biol Fertil Soils 2009, 45:563-571.

45. Barret M, Briand M, Bonneau S, Préveaux A, Valière S, Bouchez O, Hunault G, Simoneau P, Jacques M -A: Emergence shapes the structure of the seed microbiota. Appl Environ Microbiol 2015, 81:1257-1266.

46. Shetty A A, Rana R, Buckseth T, Preetham S P: Waste utilization in cucurbits: A review. Waste Biomass Valorization 2012, 3:363-368.

47. Jacks T J, Hensarling T P, Yatsu L Y: Cucurbit seeds: I. Characterization and uses of oils and proteins. A review. Econ Bot 1971:135-141.

48. The United States Department of Agriculture (USDA) National Nutrient Database for Standard Reference. 49. Bankole S A, Adenusi A A, Lawal O S, Adesanya O O: Occurrence of aflatoxin B1 in food products derivable from “egusi” melon seeds consumed in southwestern Nigeria. Food Control 2010, 21:974-976.

50. Munsell H E, Williams L O, Guild L P, Troescher C B, Nightingale G, Harris R S: Composition of food plants of Central America. III. Guatemala. J Food Sci 1950, 15:34-52.

51. Maheshwari D K (Ed): Plant Growth and Health Promoting Bacteria. Volume 18. Berlin, Heidelberg: Springer Berlin Heidelberg; 2011. [Microbiology Monographs] 52. Borriss R: Use of plant-associated Bacillus strains as biofertilizers and biocontrol agents in agriculture. In Bacteria in agrobiology: Plant growth responses. Edited by Maheshwari D K. Berlin, Heidelberg: Springer Berlin Heidelberg; 2011.

53. McSpadden Gardener B B, Fravel D R: Biological control of plant pathogens: research, commercialization, and application in the USA. Plant Health Prog 2002.

54. Lacey L A, Frutos R, Kaya H K, Vail P: Insect pathogens as biological control agents: Do they have a future? Biol Control 2001, 21:230-248.

55. Mundt J O, Hinkle N F: Bacteria within ovules and seeds. Appl Environ Microbiol 1976, 32:694-698.

56. Mihong G, Gang C, Qing L G, Juan H, Xiang H, Gong Z L, Lei W, Xia Y L: Antagonism watermelon Fusarium wilt of endophytic bacteria screening and identification. Int J Digit Content Technol Its Appl 2013, 7:1097.

57. Nga N T T, Giau N T, Long N T, Lubeck M, Shetty N P, de Neergaard E, Thuy T T T, Kim P V, Jørgensen H J L: Rhizobacterially induced protection of watermelon against Didymella bryoniae. J Appl Microbiol 2010.

58. Yun, L, Ming L: Isolation of endophytic bacteria from hami melon and screening of antagonistic bacteria. Journal of Shihezi University (Natural Science) 2004:-S1.

59. Cao Y, Zhang Z, Ling N, Yuan Y, Zheng X, Shen B, Shen Q: Bacillus subtilis SQR 9 can control Fusarium wilt in cucumber by colonizing plant roots. Biol Fertil Soils 2011, 47:495-506.

60. Li L, Ma J, Li Y, Wang Z, Gao T, Wang Q: Screening and partial characterization of Bacillus with potential applications in biocontrol of cucumber Fusarium wilt. Crop Prot 2012, 35:29-35.

61. Romero D, Pérez-Garcia A, Rivera M E, Cazorla F M, de Vicente A: Isolation and evaluation of antagonistic bacteria towards the cucurbit powdery mildew fungus Podosphaera fusca. Appl Microbiol Biotechnol 2004, 64:263-269.

62. Trutmann P, Keane P J, Merriman P R: Reduction of sclerotial inoculum of Sclerotinia sclerotiorum with Coniothyrium minitans. Soil Biol Biochem 1980, 12:461-465.

63. Tsavkelova E A, Cherdyntseva T A, Netrusov A I: Auxin production by bacteria associated with orchid roots. Microbiology 2005, 74:46-53.

64. Zimmer W, Hundeshagen B, Niederau E: Demonstration of the indolepyruvate decarboxylase gene homologue in different auxin-producing species of the Enterobacteriaceae. Can J Microbiol 1994, 4:1072-1076.

65. Tsavkelova E A, Cherdyntseva T A, Netrusov A I: Auxin production by bacteria associated with orchid roots. Microbiology 2005, 74:46-53.

66. Lal S, Tabacchioni S: Ecology and biotechnological potential of Paenibacillus polymyxa: a minireview. Indian J Microbiol 2009, 49:2-10.

67. Lemaire-Chamley M: Changes in transcriptional profiles are associated with early fruit tissue specialization in tomato. Plant Physiol 2005, 139:750-769.

68. Nuss E T, Tanumihardjo S A: Maize: A paramount staple crop in the context of global nutrition. Compr Rev Food Sci Food Saf 2010, 9:417-436.

69. Hudson C, Hudson S P, Hecht T, MacKenzie J: Protein source tryptophan versus pharmaceutical grade tryptophan as an efficacious treatment for chronic insomnia. Nutr Neurosci 2005,8:121-127.

70. Whitaker T W: Archeological cucurbits. Econ Bot 1981, 35:460-466.

71. Postma J A, Lynch J P: Complementarity in root architecture for nutrient uptake in ancient maize/bean and maize/bean/squash polycultures. Ann Bot 2012, 110:521-534.

72. Lakshmi J A, Kaul P: Nutritional potential, bioaccessibility of minerals and functionality of watermelon (Citrullus vulgaris) seeds. LWT—Food Sci Technol 2011, 44:1821-1826.

73. Asiegbu JE: Some biochemical evaluation of fluted pumpkin seed. J Sci Food Agric 1987, 40:151-155.

74. Wani A A, Sogi D S, Singh P, Wani I A, Shivhare U S: Characterisation and functional properties of watermelon (Citrullus lanatus) seed proteins. J Sci Food Agric 2011, 91:113-121.

75. Hudson S P, Hudson C J: Defatted meal from squash seeds containing protein-bound tryptophan and carbohydrate source (glucose, maltose, sucrose) for facilitating transport across the blood brain barrier; useful for inducing sleep. 2003.

76. Smith C R, Shekleton M C, Wolff I A, Jones Q: Seed protein sources—amino acid composition and total protein content of various plant seeds. Econ Bot 1959, 13:132-150.

77. Abeles F B, Morgan P W, Saltveit M E: Ethylene in Plant Biology. 2nd Edition. San Diego: Academic Press, Inc.; 1992.

78. Saleem M, Arshad M, Hussain S, Bhatti A S: Perspective of plant growth promoting rhizobacteria (PGPR) containing ACC deaminase in stress agriculture. J Ind Microbiol Biotechnol 2007, 34:635-648.

79. Wahab A A: Nitrogen fixation by Bacillus strains isolated from the rhizosphere of Ammophila arenaria. Plant Soil 1975, 42:703-708.

80. Seldin L, Van Elsas J D, Penido E G C: Bacillus azotofixans sp. nov., a nitrogen-fixing species from Brazilian soils and grass roots. Int J Syst Bacteriol 1984, 34:451-456.

81. Coelho M R R, Weid I, Zahner V, Seldin L: Characterization of nitrogen-fixing Paenibacillus species by polymerase chain reaction-restriction fragment length polymorphism analysis of part of genes encoding 16S rRNA and 23S rRNA and by multilocus enzyme electrophoresis. FEMS Microbiol Lett 2003, 222:243-250.

82. Weyens N, van der Lelie D, Taghavi S, Newman L, Vangronsveld J: Exploiting plant-microbe partnerships to improve biomass production and remediation. Trends Biotechnol 2009, 27:591-598.

83. Zlotnikov K M, Zlotnikov A K, Kaparullina E N, Doronina N V: Phylogenetic position and phosphate solubilization activity of lactic acid bacteria associated with different plants. Microbiology 2013, 82:393-396.

84. Rodriguez H, Fraga R, Gonzalez T, Bashan Y: Genetics of phosphate solubilization and its potential applications for improving plant growth-promoting bacteria. Plant Soil 2006, 287:15-21.

85. Duijff B J, Recorbet G, Bakker P A, Loper J E, Lemanceau P: Microbial antagonism at the root level is involved in the suppression of Fusarium wilt by the combination of nonpathogenic Fusarium oxysporum Fo47 and Pseudomonas putida WCS358. Phytopathology 1999, 89:1073-1079.

86. Fiedler H -P, Krastel P, Muller J, Gebhardt K, Zeeck A: Enterobactin: the characteristic catecholate siderophore of Enterobacteriaceae is produced by Streptomyces species. FEMS Microbiol Lett 2001, 196:147-151.

87. Chen X H, Koumoutsi A, Scholz R, Eisenreich A, Schneider K, Heinemeyer I, Morgenstern B, Voss B, Hess W R, Reva O, Junge H, Voigt B, Jungblut P R, Vater J, Sussmuth R, Liesegang H, Strittmatter A, Gottschalk G, Borriss R: Comparative analysis of the complete genome sequence of the plant growth—promoting bacterium Bacillus amyloliquefaciens FZB42. Nat Biotechnol 2007, 25:1007-1014.

88. Paungfoo-Lonhienne C, Lonhienne T G, Rentsch D, Robinson N, Christie M, Webb R I, Gamage H K, Carroll B J, Schenk P M, Schmidt S: Plants can use protein as a nitrogen source without assistance from other organisms. Proc Nati Acad Sci 2008, 105:4524-4529.

89. Sessitsch A, Hardoim P, Döring J, Weilharter A, Krause A, Woyke T, Mitter B, Hauberg-Lotte L, Friedrich F, Rahalkar M, others: Functional characteristics of an endophyte community colonizing rice roots as revealed by metagenomic analysis. Mol Plant Microbe Interact 2012, 25:28-36.

90. Derrien M, van Hylckama Vlieg J E T: Fate, activity, and impact of ingested bacteria within the human gut microbiota. Trends Microbiol 2015, 23:354-366.

91. Galland L: The gut microbiome and the brain. J Med Food 2014, 17:1261-1272.

92. Di Cagno R, Coda R, De Angelis M, Gobbetti M: Exploitation of vegetables and fruits through lactic acid fermentation. Food Microbiol 2013, 33:1-10.

93. Preidis G A, Versalovic J: Targeting the Human Microbiome With Antibiotics, Probiotics, and Prebiotics: Gastroenterology Enters the Metagenomics Era. Gastroenterology 2009, 136:2015-2031.

94. Cutting S M: Bacillus probiotics. Food Microbiol 2011, 28:214-220.

95. Phalip V, Schmitt P, Divies C: A method for screening diacetyl and acetoin-producing bacteria on agar plates. J Basic Microbiol 1994, 34:277-280.

96. Hole R C, Singhal R S, Melo J S, D'Souza S F: A rapid plate screening technique for extracellular ribonuclease producing strains. BARC Newsl 2004, 249:91-97.

97. Amend A S, Seifert K A, Samson R, Bruns T D: Indoor fungal composition is geographically patterned and more diverse in temperate zones than in the tropics. Proc Natl Acad Sci 2010, 107:13748-13753.

98. Sun Z -B, Yuan X -F, Zhang H, Wu L -F, Liang C, Feng Y -J: Isolation, screening and identification of antagonistic downy mildew endophytic bacteria from cucumber. Eur J Plant Pathol 2013,137:847-857.

99. Pramanik M H R, Nagai M, Asao T, Matsui Y: Effects of temperature and photoperiod on phytotoxic root exudates of cucumber (Cucumis sativus) in hydroponic culture. J Chem Ecol 2000, 26:1953-1967.

100. Kim J J, Goettel M S, Gillespie D R: Potential of Lecanicillium species for dual microbial control of aphids and the cucumber powdery mildew fungus, Sphaerotheca fuliginea. Biol Control 2007, 40:327-332.

1. An isolated bacterial endophyte comprising:
a 16S rDNA gene comprising a nucleotide sequence with at least 95% sequence identity to SEQ ID NO: 60, wherein the bacterial endophyte is of the genus Pediococcus.

2. The bacterial endophyte of claim 1, wherein the endophyte comprises a 16S rDNA gene comprising the nucleotide sequence of SEQ ID NO: 60.

3. The bacterial endophyte of claim 1, wherein the endophyte inhibits the growth of powdery mildew, optionally Podosphaera fulginea.

4. The bacterial endophyte of claim 1, wherein the endophyte inhibits the growth of Rhizoctonia solani and/or Phytophthora capsici, and/or has phosphate solubilization activity.

5. A composition comprising a population of the isolated bacterial endophyte of claim 1 and at least one of a carrier, tackifier, microbial stabilizer, fungicide, antibacterial agent, herbicide, nematicide, insecticide, plant growth regulator, rodenticide, dessicant, nutrient and culture media.

6. The composition of claim 5, wherein the carrier comprises an agricultural carrier, a surfactant or a seed coating agent.

7. The composition of claim 5, composition is a bacterial cell culture comprising at least 10³ colony forming units (CFU) per ml.

8. The composition of claim 5, wherein the composition is in the form of a fluid or powder suitable for inoculating a plant with the bacterial endophyte.

9. A synthetic combination comprising the bacterial endophyte of claim 1 in association with a plant, wherein the bacterial endophyte is heterologous to the plant.

10. The synthetic combination of claim 9, werein the bacterial endophyte is in association with a seed or seedling.

11. The synthetic combination of claim 10, wherein the bacterial endophyte is present in the synthetic combination at a concentration of at least 10³ CFU per seed.

12. The synthetic combination of claim 9 wherein the bacterial endophyte is present in the synthetic combination at a concentration sufficient to inhibit the growth of powdery mildew in a plant produced from the seed relative to a plant produced from a seed that is not in association with the bacterial endophyte.

13. A method of preventing or inhibiting growth of powdery mildew on a plant, comprising inoculating the plant with a bacterial endophyte identified in Table 7, 8 or 9.

14. The method of claim 13, wherein the bacterial endophyte is heterologous to the plant.

15. The method of claim 13, wherein the bacterial endophyte is selected from EKM101V, EKM102Z, EKM103V, EKM201S, EKM301K, EKM201D, EKM505B and EKM419B.

16. The method of claim 13, wherein the powdery mildew is Podosphaera fulginea.

17. The method of claim 13, wherein the plant is an agricultural plant, optionally a fruit or vegetable.

18. The method of claim 13, wherein inoculating the plant comprises coating the seeds of the plant and/or exposing the plant to a spray.

19. The method of claim 13, wherein inoculating the plant with the bacterial endophyte inhibits the growth of a fungal pathogen selected from Fusarium graminearum, Rhizoctonia solani, Phytophthora capsici and Pythium aphanideratum or the plant.

20. A method for preparing an agricultural seed composition, comprising contacting the surface of a plurality of seeds with a formulation comprising a bacterial endophyte identified in Table 7, 8 or 9, wherein the endophyte is present on the seeds in an amount effective to inhibit the growth of powdery mildew on the seeds or plants derived from the seeds.
